(19) United States
(12) Patent Application Publication
(10) Pub. No.: US 2013/0243808 A1
(43) Pub. Date: Sep. 19, 2013

(54) COMPOSITIONS AND METHODS FOR VACCINATING HUMANS AND ANIMALS AGAINST ENVELOPED VIRUSES

(71) Applicant: Olga Baer, (US)

(72) Inventor: George Baer, Walkill, NY (US)

(73) Assignee: Olga Baer, Walkill, NY (US)

(21) Appl. No.: 13/651,921

(22) Filed: Oct. 15, 2012

Related U.S. Application Data
(63) Continuation-in-part of application No. PCT/US2011/032719, filed on Apr. 15, 2011.
(60) Provisional application No. 61/324,703, filed on Apr. 15, 2010.

(51) Int. Cl. A61K 39/145

(52) U.S. Cl. CPC A61K 39/145
USPC 424/192.1; 424/209.1; 435/236

(57) ABSTRACT

Immunogenic compositions and methods for vaccinating humans and animals against enveloped viruses, and more particularly against influenza viruses. Immunogenic compositions are composed of internal moieties of an enveloped virus. Such internal moieties are more highly conserved between different viral strains than are their external moiety counterparts. Consequently, these immunogenic compositions have an increased likelihood of generating immuno-protective responses in humans and animals against enveloped viruses that are strain independent. Methods are also disclosed that increase the ability of the immunogenic compositions to generate immuno-protective responses against their target viruses.

### CROSS-REFERENCE TO RELATED APPLICATION

This application claims the benefit of PCT Application No. PCT/US2011/032719, filed Apr. 15, 2011, which claims the benefit of U.S. Provisional Application No. 61/324,703, filed Apr. 15, 2010, the entire contents of both applications are incorporated herein by reference.

### BACKGROUND OF THE INVENTION

Eukaryotic viruses represent a large and diverse group of infectious agents known for the wide variety of diseases they cause. Based on their viral particle structure, viruses may be broadly classified as being either “non-enveloped” or “enveloped.” Non-enveloped viruses contain a viral genome that is surrounded by a proteinaceous capsid, which is formed by products encoded by the viral genome and synthesized by the machinery of the host cell. Enveloped viruses also have a viral genome surrounded by a proteinaceous capsid, however, the capsid is further encapsulated within an “envelope” comprised of a protein containing phospholipid bilayer. This envelope is acquired as the viral capsid buds through the cell membrane, and is usually derived from the outer membrane of the host cell, but may also be derived from the nuclear membrane, Golgi apparatus, or endoplasmic reticulum.

Enveloped viruses represent a diverse family of viruses that include, but are not limited to, arenaviridae, arteriviridae, asfarviridae, baculoviridae, bornaviridae, bunyaviridae, coronaviridae, filoviridae, flaviviridae, hepadnaviridae, herpesviridae, orthomyxoviridae, paramyxoviridae, poxyiridae, retroviridae, rhabdoviridae, and togaviridae. These virus families are responsible for a wide variety of human and animal diseases including, but not limited to, encephalitis, gastro-intestinal disease, hemorrhagic disease, hepatitis, immunosuppressive diseases, ocular disease, pox (e.g. chickenpox, cowpox, smallpox, monkeypox, felinepox, swinepox, and pseudo-cowpox), respiratory disease, sexually transmitted disease, and cancer, and result in billions of infections, and millions of deaths, world wide every year. For example, infections with influenza viruses in humans are a common and significant cause of respiratory disease and result in an average of approximately 20,000 deaths and 114,000 hospitalizations per year in the U.S. alone. Severe outbreaks of influenza periodically affect millions of people. In one instance, the 1918-1919 Spanish flu pandemic was responsible for killing an estimated 20 million people. In another instance, 80% of the U.S. Army's casualties during World War I were attributed to influenza.

Vaccinating humans and animals against influenza viruses is difficult because the virally encoded proteins present in the envelope, which are the primary targets of the immune system, undergo significant antigenic variation as a result of the processes of antigenic drift and antigenic shift. Antigenic “drift” involves minor antigenic changes in the envelope proteins hemagglutinin (HA) and neuraminidase (NA), while antigenic “shift” involves major antigenic changes in these molecules. Changes in the conformation of these two antigens are accompanied by changes in antigenicity, which facilitates the ability of the viral particle to evade detection by the immune system. The combination of antigenic drift and shift allows the genetic constitution of influenza particles to change very rapidly.

In the United States vaccines are prepared each year for the annual vaccination program, with the quantity of immunoreactive HA in each dose standardized to contain the amount recommended by the Bureau of Biologics, U.S. Food and Drug Administration (FDA). The resistance to infection after vaccine administration correlates with the level of serum antibody produced to the specific strain active that year. In other words, antigenic changes within the HA and NA subtypes described above require concomitant changes in the vaccine, according to the type(s) of antigen present.

The challenge of vaccinating human and animal populations is further complicated by the fact that there is a growing complexity of influenza viruses operating at the human-animal interface. For example, the isolation of viruses with a seemingly high affinity for reassortment indicates that the U.S. swine population is an increasingly important reservoir of viruses that may have human pandemic potential. Recently, multiple lineages of antigenically and genetically diverse swine influenza viruses (SIV) have been identified in U.S. swine populations. As another example, reassortment between human and avian influenza viruses produced the strains that caused both the 1957 and 1968 human influenza pandemics. Based on evidence from the three pandemics that occurred during the 20th century, scientists have determined that pandemic flu strains tend to infect between 25% and 35% of the population. In September 2005, the World Health Organization (WHO) estimated that a new influenza pandemic would likely result in the death of between 2 million and 7 million people. New disease challenges such as novel reassorted viruses and the use of viruses as bioterrorism agents can be devastating for both animal and human populations. Therefore there is an urgent need for technologies that can enhance the immune system, prevent or greatly reduce the shed of virus, and improve clinical outcome of the disease in animal and human populations in order to help reduce the pandemic potential of enveloped viruses, and influenza viruses in particular.

### SUMMARY OF THE INVENTION

The present disclosure relates to compositions and methods for vaccinating humans and animals against enveloped viruses, and more particularly against influenza viruses.

In one aspect, the invention provides an immunogenic composition that includes an effective amount of at least one internal moiety of an enveloped virus, or antigenic fragment thereof, and a pharmaceutically acceptable carrier or excipient.

In one embodiment, the enveloped virus is from the arenaviridae family, the arteriviridae family, the asfarviridae family, the baculoviridae family, the bornaviridae family, the bunyaviridae family, the coronaviridae family, the filoviridae family, the flaviviridae family, the hepadnaviridae family, the herpesviridae family, the orthomyxoviridae family, the paramyxoviridae family, the poxyiridae family, the retroviridae family, the rhabdoviridae family, and the togaviridae family. In a preferred embodiment, the enveloped virus is influenza.

In one embodiment, the internal moiety is any one or more viral polypeptides or polynucleotides selected from the group consisting of the RNA genome (RNAg), nucleoprotein (NP), RNA polymerase (PA), RNA polymerase 1 (PB 1), RNA polymerase 2 (PB2), non-structural protein 1 (NS1), non-structural protein 2 (NS2), and matrix protein 1 (M1).

In a preferred embodiment, the at least one internal moiety includes NP. In another preferred embodiment, the at least one internal moiety includes NP and RNAg. In yet another preferred embodiment, the at least one internal moiety is NP. In another preferred embodiment, the at least one internal moiety is NP and RNAg.

In another aspect, the invention provides an immunogenic composition that includes an effective amount of a naked viral core particle of an enveloped virus and a pharmaceutically acceptable carrier or excipient.

In one embodiment, the enveloped virus is from the arenaviridae family, the arteriviridae family, the asfarviridae family, the baculoviridae family, the bornaviridae family, the bunyaviridae family, the coronaviridae family, the filoviridae family, the flaviviridae family, the hepadnaviridae family, the herpesviridae family, the orthomyxoviridae family, the paramyxoviridae family, the poxyiridae family, the retroviridae family, the rhabdoviridae family, and the togaviridae family. In a preferred embodiment, the enveloped virus is influenza.

In one embodiment, the naked viral core particle includes any one or more viral polypeptides or polynucleotides from the following group: the RNA genome (RNAg), nucleoprotein (NP), RNA polymerase (PA), RNA polymerase 1 (PB 1), RNA polymerase 2 (PB2), non-structural protein 1 (NS1), and non-structural protein 2 (NS2).

In one embodiment, the naked viral core particle includes at least three viral polypeptides or polynucleotides from the following group: RNAg, NP, PA, PB1, PB2, NS1, and NS2. In a preferred embodiment, the naked viral core particle contains NP, NS1, and RNAg. In another preferred embodiment, the naked viral core particle contains NP, NS2, and RNAg. In yet another preferred embodiment, the naked viral core particle contains NP, NS1, NS2, and RNAg.

In another aspect, the invention provides an immunogenic composition comprising an effective amount of at least one virus like particle (VLP) of an enveloped virus and a pharmaceutically acceptable carrier or excipient.

In one embodiment, the enveloped virus is from the arenaviridae family, the arteriviridae family, the asfarviridae family, the baculoviridae family, the bornaviridae family, the bunyaviridae family, the coronaviridae family, the filoviridae family, the flaviviridae family, the hepadnaviridae family, the herpesviridae family, the orthomyxoviridae family, the paramyxoviridae family, the poxyiridae family, the retroviridae family, the rhabdoviridae family, and the togaviridae family. In a preferred embodiment, the enveloped virus is influenza.

In one embodiment, the VLP includes any one or more viral polypeptides or polynucleotides, or antigenic fragments thereof, selected from the group consisting of RNA genome (RNAg), nucleoprotein (NP), RNA polymerase (PA), RNA polymerase 1 (PB1), RNA polymerase 2 (PB2), non-structural protein 1 (NS1), and non-structural protein 2 (NS2). In another embodiment, the antigenic fragment is an N-terminal, a C-terminal, or an internal polypeptide fragment.

In another aspect, the invention provides an immunogenic composition comprising an effective amount of at least one recombinant fusion protein, or an antigenic fragment thereof, of an enveloped virus and a pharmaceutically acceptable carrier or excipient. In one embodiment, the at least one recombinant fusion protein includes a protein from the following group: nucleoprotein (NP), RNA polymerase (PA), RNA polymerase 1 (PB 1), RNA polymerase 2 (PB2), non-structural protein 1 (NS1), non-structural protein 2 (NS2), and/or matrix protein 1 (M1).

In one embodiment, the at least one recombinant fusion protein is joined with a viral polypeptide, or antigenic fragment thereof, selected from the group of viral proteins that includes: hemagglutinin (HA), neuraminidase (NA), and matrix protein 2 (M2). In another embodiment, the at least one recombinant fusion protein is NP, or an antigenic fragment thereof, joined to HA, or an antigenic fragment thereof.

In another aspect, the invention provides a method of synthesizing naked viral core particles from enveloped viruses that involves producing live virus from a host, purifying the live virus from the host, and removing the envelope from the purified live virus.

In another aspect, the invention provides a method of synthesizing naked viral core particles from enveloped viruses that involves producing live virus from a cell culture system, purifying the live virus from the cell culture system, and removing the envelope from the purified live virus.

In another aspect, the invention provides a method of synthesizing naked viral core particles that involves producing in vitro the genome and protein components of the virus of interest corresponding to the desired internal moieties.

In another aspect, the invention provides a method of inducing immunity against an enveloped virus in a mammal that involves administering the mammal with an immunologically effective amount of an immunogenic composition as described above. In one embodiment, the method of administration is selected from the group consisting of topical, oral, anal, vaginal, mucosal, intravenous, intraperitoneal, intramuscular, subcutaneous, intranasal, nasopharyngeal, buccal, and intradermal administration. In a preferred embodiment, the method of administration is intradermal.

In another aspect, the invention provides a method of generating an immune response in a subject, the method comprising administering to said mammal an antigenic amount of any of the above described immunogenic compositions.

In another aspect, the invention provides an influenza vaccine that includes a therapeutically or prophylactically effective amount of a naked viral core particle, which may include any combination of one or more of the RNA genome (RNAg), nucleoprotein (NP), RNA polymerase (PA), RNA polymerase 1 (PB 1), RNA polymerase 2 (PB2), non-structural protein 1 (NS1), and non-structural protein 2 (NS2); a pharmaceutically acceptable carrier or excipient; and, a pharmaceutically acceptable adjuvant.

In another aspect, the invention provides a method of vaccinating a mammal against influenza that involves administering to the mammal intradermally a vaccine including: a therapeutically or prophylactically effective amount of a naked viral core particle, which may include any combination of one or more of the RNA genome (RNAg), nucleoprotein (NP), RNA polymerase (PA), RNA polymerase 1 (PB 1), RNA polymerase 2 (PB2), non-structural protein 1 (NS1), and non-structural protein 2 (NS2); a pharmaceutically acceptable carrier or excipient; and, a pharmaceutically acceptable adjuvant.

The present disclosure provides compositions comprised of naked viral core particles that recapitulate the internal moieties of an enveloped virus, including the viral genome and its genome associated proteins. Advantageously, such internal moieties generally display higher levels of amino acid conservation between strains than the external moieties associated with viral envelope proteins; therefore, the use of vaccine compositions comprising such internal moieties may provide immuno-protection against enveloped viruses that is independent of strain variation.

The present disclosure also provides compositions that are derived from naked viral core particles, including: virus like particles (viral genome in combination with one or more internal moieties), internal viral proteins (purified internal moieties, individually or in combination), derivative internal viral proteins (deletion derivatives of internal moieties, alone or in combination), and recombinant fusion proteins (internal moieties fused with extrinsic proteins or protein fragments).

The present disclosure further relates to a method of administering compositions containing naked viral core particles or naked viral core particle derivatives via an intradermal route.

### DEFINITIONS

By “enveloped virus” is meant any virus in which the capsid is encapsulated within a phospholipid bilayer. The bilayer may, or may not, contain host and/or virally encoded proteins. For example, enveloped viruses may belong to the family of viruses that includes, but is not limited to, arenaviridae, arteriviridae, asfarviridae, baculoviridae, bornaviridae, bunyaviridae, coronaviridae, filoviridae, flaviviridae, hepadnaviridae, herpesviridae, orthomyxoviridae, paramyxoviridae, poxyiridae, retroviridae, rhabdoviridae, and togaviridae.

By “influenza virus” is meant any virus belonging to the orthomyxoviridae genera. Exemplary influenza viruses include, but are not limited to, influenza A, influenza B, and influenza C. Of these, the most virulent human pathogen is influenza A, which is a negative stranded, segmented, enveloped RNA virus comprised of a helical ribonucleocapsid known as the viral ribonucleoprotein (vRNP). The vRNP contains a genome comprised of eight negative sense RNA segments that encode eleven viral proteins, including: hemagglutinin (HA), neuraminidase (NA), nucleoprotein (NP), M1, M2, NS1, NS2 (aka NEP), PA, PB1, PB1-F2, and PB2. The genome segments are packed in a helical form with NP, and the resulting RNP structures are associated with the three individual subunits (PB1, PB2, and PA) of the viral polymerase, which is known as the 3P complex in its assembled form. The vRNP may also be associated with NS2. Viral particles are packaged when RNPs accumulate at the cell surface near regions of the host cell membrane that have accumulated the viral transmembrane envelope proteins, HA, NA, and M2. The virus particle acquires its envelope as it buds from the host cell. HA trimers and NA tetramers display prominent glycoprotein spikes on the exterior surface of the envelope, with HA being the predominant envelope protein. The M2 protein encodes an ion channel, and is present in the envelope at a very low concentration estimated to be 16-20 molecules per virion. The viral protein M1 is located on the interior surface of the viral envelope, and is believed to play a role in linking the internal domains of HA and NA with the RNP. The morphology of influenza viral particles is highly variable, ranging from discrete spherical particles to filamentous particles that can be up to 2000 nm long. The most striking feature of influenza virions is the dense layer of HA and NA spikes projecting radially outward over the surface of the viral envelope, and the epitopes encoded by these spikes represent important targets for the immune system of the host (e.g. animal or human).

Influenza subtypes include, but are not limited to, fifteen HA subtypes (H1-H15) and nine NA subtypes (N1-N9) that have been identified to date. Characterized influenza A subtypes include, but are not limited to, H1N1 (1918 Spanish flu pandemic), H2N2 (1957 Asian flu pandemic), H3N2 (1968 Hong Kong flu pandemic), H5N1 (current pandemic threat), H7N7 (current zoonotic threat), H1N2 (endemic in humans and swine), H9N2, H7N2, H7N3, and H10N7.

By “internal moiety” is meant any protein or polynucleotide component of an enveloped virus internal to the viral envelope (i.e. not present in the envelope) or antigenic fragment thereof. For example, internal moieties of influenza include the viral genome and its genome associated proteins, and antigenic fragments. Exemplary internal moieties include, but are not limited to the RNAg, NP, PB1, PB2, PA, NS1, NS2, and/or M1.

By “naked viral core particle (NVCP)” is meant an enveloped virus that has been stripped of its envelope and isolated. An influenza NVCP may be comprised of the viral genome (RNAg) in combination with one or more associated proteins, but does not include the viral envelope. For example, an influenza NVCP may include RNAg, NP, PB1, PB2, PA, and/or NS2.

By “virus-like particles (VLP)” is meant a structure that in at least one attribute resembles a virus, but which has not been demonstrated to be infectious. In general, virus-like particles lack a complete viral genome and, therefore, are noninfectious. In addition, virus-like particles can often be produced in large quantities by heterologous expression and can be easily purified.

For example, an influenza VLP composition may be comprised of RNAg and NP, RNAg and PB1, RNAg and PB2, RNAg and PA, or RNAg and NS2. As another example, an influenza VLP composition may also include, but is not limited to, RNAg and NP plus PB 1, or RNAg and NP plus PB2, or RNAg and NP plus PA, or RNAg and NP plus NS2.

By “recombinant fusion protein (RFP)” is meant any fusion of two polypeptides, or fragments thereof, that are not naturally contiguous. Exemplary fusion proteins include N-terminal or C-terminal fusions of NP, PB 1, PB2, PA, and NS2 with proteins or protein fragments that may increase the immunogenicity of the resulting recombinant protein. The term also encompasses the fusion of an internal fragment of NP, PB1, PB2, PA, and NS2 with proteins or protein fragments that may increase the immunogenicity of the resulting recombinant protein. For example, candidate proteins or protein fragments for use with influenza specific compositions may include, or be derived from, hemagglutinin (HA), neuraminidase (NA), and matrix protein 2 (M2).

By “nucleoprotein (NP) nucleic acid molecule” is meant a polynucleotide encoding a NP polypeptide. An exemplary NP nucleic acid molecule is provided at NCBI Accession No. NC_{—}002019.1. Additional exemplary NP nucleic acid molecules include, but are not limited to, the following strain specific variant transcripts: variant 2, NCBI Accession No. NC_{—}007360.1 and variant 3, NCBI Accession No. NC_{—}007360.1.

By “nucleoprotein (NP) polypeptide” is meant a polypeptide, or fragment thereof, having at least about 85% amino acid identity to NP_{—}040982.1, and having nucleic acid binding activity. Additional exemplary NP polypeptide molecules include, but are not limited to, the following strain specific variants: polypeptide variant 2, NCBI Accession No. YP_{—}308667.1 and polypeptide variant 3, NCBI Accession No. YP_{—}308843.1.

By “PB1 nucleic acid molecule” is meant a polynucleotide encoding a PB1 polypeptide. An exemplary PB1 nucleic acid molecule is provided at NCBI Accession No. NC_{—}002021.1. Additional exemplary PB1 nucleic acid molecules include, but are not limited to, the following strain specific variant transcripts: variant 2, NCBI Accession No. NC_{—}007358.1 and variant 3, NCBI Accession No. NC_{—}007375.1.

By “PB1 polypeptide” is meant a polypeptide, or fragment thereof, having at least about 85% amino acid identity to NP_{—}040985.1, and having RNA polymerase activity. Additional exemplary PB 1 polypeptide molecules include, but are not limited to, the following strain specific variants: polypeptide variant 2, NCBI Accession No. YP_{—}308665.1 and polypeptide variant 3, NCBI Accession No. YP_{—}308851.1.

By “PB2 nucleic acid molecule” is meant a polynucleotide encoding a PB2 polypeptide. An exemplary PB2 nucleic acid molecule is provided at NCBI Accession No. NC_{—}002023.1. Additional exemplary PB2 nucleic acid molecules include, but are not limited to, the following strain specific variant transcripts: variant 2, NCBI Accession No. NC_{—}007357.1 and variant 3, NCBI Accession No. NC_{—}007373.1.

By “PB2 polypeptide” is meant a polypeptide, or fragment thereof, having at least about 85% amino acid identity to NP_{—}040987.1, and having RNA polymerase activity. Additional exemplary PB2 polypeptide molecules include, but are not limited to, the following strain specific variants: polypeptide variant 2, NCBI Accession No. YP_{—}308664.1 and polypeptide variant 3, NCBI Accession No. YP_{—}308849.1.

By “PA nucleic acid molecule” is meant a polynucleotide encoding a PA polypeptide. An exemplary PA nucleic acid molecule is provided at NCBI Accession No. NC_{—}002022.1. Additional exemplary PA nucleic acid molecules include, but are not limited to, the following strain specific variant transcripts: variant 2, NCBI Accession No. NC_{—}007359.1 and variant 3, NCBI Accession No. NC_{—}007376.1.

By “PA polypeptide” is meant a polypeptide, or fragment thereof, having at least about 85% amino acid identity to NP_{—}040986.1, and having RNA polymerase activity. Additional exemplary PA polypeptide molecules include, but are not limited to, the following strain specific variants: polypeptide variant 2, NCBI Accession No. YP_{—}308666.1 and polypeptide variant 3, NCBI Accession No. YP_{—}308852.1.

By “non structural protein 2 (NS2) nucleic acid molecule” is meant a polynucleotide encoding a NS2 polypeptide. An exemplary NS2 nucleic acid molecule is provided at NCBI Accession No. NC_{—}002020.1. Additional exemplary NS2 nucleic acid molecules include, but are not limited to, the following strain specific variant transcripts: variant 2, NCBI Accession No. NC_{—}004906.1 and variant 3, NCBI Accession No. NC_{—}007370.1.

By “non structural protein 2 (NS2) polypeptide” is meant a polypeptide, or fragment thereof, having at least about 85% amino acid identity to NP_{—}040983.1, and having antigenic activity. Additional exemplary NS2 polypeptide molecules include, but are not limited to, the following strain specific variants: polypeptide variant 2, NCBI Accession No. YP_{—}581750.1 and polypeptide variant 3, NCBI Accession No. YP_{—}308844.1.

By “matrix protein 1 (M1) nucleic acid molecule” is meant a polynucleotide encoding a M1 polypeptide. An exemplary M1 nucleic acid molecule is provided at NCBI Accession No. NC_{—}002016.1. Additional exemplary M1 nucleic acid molecules include, but are not limited to, the following strain specific variant transcripts: variant 2, NCBI Accession No. NC_{—}007377.1 and variant 3, NCBI Accession No. NC_{—}007363.1.

By “matrix protein 1 (M1) polypeptide” is meant a polypeptide, or fragment thereof, having at least about 85% amino acid identity to NP_{—}040978.1, and having nucleic acid binding activity. Additional exemplary M1 polypeptide molecules include, but are not limited to, the following strain specific variants: polypeptide variant 2, NCBI Accession No. YP_{—}308854.1 and polypeptide variant 3, NCBI Accession No. YP_{—}308671.1.

By “non structural protein 1 (NS1) nucleic acid molecule” is meant a polynucleotide encoding a NS2 polypeptide. An exemplary NS1 nucleic acid molecule is provided at NCBI Accession No. NC_{—}002020.1. Additional exemplary NS1 nucleic acid molecules include, but are not limited to, the following strain specific variant transcripts: variant 2, NCBI Accession No. NC_{—}007370.1 and variant 3, NCBI Accession No. NC_{—}004906.1.

By “non structural protein 1 (NS1) polypeptide” is meant a polypeptide, or fragment thereof, having at least about 85% amino acid identity to NP_{—}040984.1, and having nucleic acid binding activity. Additional exemplary NS1 polypeptide molecules include, but are not limited to, the following strain specific variants: polypeptide variant 2, NCBI Accession No. YP_{—}308845.1 and polypeptide variant 3, NCBI Accession No. NP_{—}859034.1.

By “RNAg” is meant the nucleic acid segments, or subsets thereof, that comprise the viral genome and encode the viral polypeptides. An exemplary RNAg, or subset thereof, has at least 85%, 90%, 95%, or 100% identity to CY080633.1, CY080632.1, CY080631.1, CY080626.1, CY080629.1, CY080628.1, CY080627.1, and CY080630.1

As used herein, the singular forms “a”, “an”, and “the” include plural forms unless the context clearly dictates otherwise.

Unless specifically stated or obvious from context, as used herein, the term “about” is understood as within a range of normal tolerance in the art, for example within 2 standard deviations of the mean. The term “about” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

As used herein, the terms “comprises,” “comprising,” “containing,” “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “including” is used herein to mean, and is used interchangeably with, the phrase “including, but not limited to.”

Unless specifically stated or obvious from context, as used herein, the term “or” is understood to be inclusive.

The term “adjuvant” as used herein refers to a compound or mixture that enhances the immune response and/or promotes the proper rate of absorption following inoculation, and, as used herein, encompasses any uptake-facilitating agent. Acceptable adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and others. The term refers to a compound or mixture that enhances the immune response and/or promotes the proper rate of absorption following inoculation, and, as used herein, encompasses any uptake-facilitating agent. Acceptable adjuvants include, but are not limited to, complete Freund's adjuvant, incomplete Freund's adjuvant, saponin, mineral gels such as aluminum hydroxide, alum, surface active substances such as lysolecithin, pluronic polyols, polyanions, peptides, oil or hydrocarbon emulsions, keyhole limpet hemocyanins, dinitrophenol, monophosphoryl lipid A, and others.

By “alteration” is meant any change in a nucleic acid or amino acid sequence relative to a reference sequence.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

The term “capsid” is meant to refer to the protein shell of the virus. In embodiments, the capsid refers to the protein shell of the orthomyxovirus. A viral capsid may consist of multimers of oligomeric protein subunits.

By “disease” is meant any condition or disorder that damages or interferes with the normal function of a cell, tissue, or organ.

The term “epithelial surface” is meant to refer to a continuous sheet of one or more cellular layers that lines a vertebrate body compartment. An epithelial surface can be the skin. Epithelial surfaces according to certain embodiments of the invention can be cervicovaginal, oral, nasal, penile, anal, epidermal and respiratory surfaces.

The term “expression vector” is meant to refer to a vector, such as a plasmid or viral particle, which is capable of promoting expression of a foreign or heterologous nucleic acid incorporated therein. In embodiments, the nucleic acid to be expressed is “operably linked” to a promoter and/or enhancer, and is subject to transcription regulatory control by the promoter and/or enhancer.

The term “fragment” is meant to refer to a portion of a protein or nucleic acid that is substantially identical to a reference protein or nucleic acid. In embodiments, the fragment is a fragment of a gene. In embodiments, the fragment is a fragment of a viral gene. In embodiments, the fragment is a fragment of a viral protein. In embodiments, the portion can retain at least 50%, 75%, or 80%, or more preferably 90%, 95%, or even 99% of the biological activity of the reference protein or nucleic acid described herein. In other embodiments, the fragment comprises at least 5, 10, 11, 12, 13, 14, 15, 16, 17, 18, 19, or 20 amino acids of a reference protein or is a nucleic acid molecule encoding such a fragment.

The term “host” as used herein refers to an animal, preferably a mammal, and most preferably a human. In embodiments, the term host cell refers to a cell that contains a heterologous nucleic acid, such as a vector, and supports the replication or expression of the nucleic acid. In certain examples, host cells can be prokaryotic cells such as E. coli, or eukaryotic cells such as yeast, insect, amphibian, avian or mammalian cells, including human and porcine cells. Exemplary host cells include, but are not limited to, 293TT, 293ORF6, PERC.6, CHO, HEp-2, HeLa, BSC40, Vero, BHK-21, 293, C12 immortalized cell lines and primary mouse or human dendritic cells.

By “immunogenic composition” is meant an agent that induces an immune response in a subject.

By “immune response” is meant any cellular or humoral response against an antigen.

The term “in combination” in the context of the administration of immunogenic compositions is meant to refer to the use of more than one immunogenic agent. In embodiments, an immunogenic composition and an agent or treatment to disrupt an epithelial surface are administered. The use of the term “in combination” does not restrict the order in which agents or therapies are administered to a subject with an infection. A first agent or therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second agent or therapy to a subject. Any additional agent or therapy can be administered in any order with the other additional treatments. Non-limiting examples of therapies that can be administered in combination with the immunogenic compositions of the invention include analgesic agents, anesthetic agents, antibiotics, or immunomodulatory agents or any other agent listed in the U.S. Pharmacopoeia and/or Physician's Desk Reference.

By “isolated nucleic acid molecule” is meant a nucleic acid (e.g., a DNA) that is free of the genes that, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences.

“Live attenuated virus” or “attenuated recombinant virus” refer to a virus that has been genetically altered by modern molecular biological methods, e.g., restriction endonuclease and ligase treatment, and rendered less virulent than wild type virus. In embodiments, attenuation results from deletion of specific genes, by serial passage in a non-natural host cell line, or by serial passage at cold temperatures. In embodiments, the live attenuated virus is a live attenuated influenza virus.

The term “nucleic acid” or “nucleic acid segment” is meant to refer to an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases. A nucleic acid segment can include a gene.

The term “pharmaceutically acceptable” as used herein means being approved by a regulatory agency of the Federal or a state government, or listed in the U.S. Pharmacopia, European Pharmacopia or other generally recognized pharmacopia for use in animals, e.g., humans.

“Pharmaceutically acceptable excipient, carrier or adjuvant” refers to an excipient, carrier or adjuvant that can be administered to a subject, together with an agent, and which does not destroy the pharmacological activity thereof and is nontoxic when administered in doses sufficient to deliver a therapeutic amount of the agent.

By “polypeptide” is meant any chain of amino acids, regardless of length or post-translational modification.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition, e.g., viral infection.

“Potentiating” or “enhancing” an immune response means increasing the magnitude and/or the breadth of the immune response, e.g., the number of cells induced by a particular epitope may be increased and/or the numbers of epitopes that are recognized may be increased. Preferably, the immune response is enhanced 2-fold, 3-fold, 4-fold, 5-fold, 6-fold, 7-fold, 8-fold, 9-fold, 10-fold, or greater after administration of the immunogenic compositions described herein. Such enhancement may also be measured relative to a control subject where the immune response was not enhanced. In one embodiment, such an “enhanced immune response” refers to an increase in the immune response in a subject by at least 10% relative to a control subject where the immune response was not enhanced. Preferably, the immune response is increased by 25%, 50%, 75% or 100%.

The term “promoter” refers to a DNA sequence that is recognized by RNA polymerase and initiates transcription.

A “retrovirus” is a virus containing an RNA genome and an enzyme, reverse transcriptase, which is an RNA-dependent DNA polymerase that uses an RNA molecule as a template for the synthesis of a complementary DNA strand. The DNA form of a retrovirus commonly integrates into the host-cell chromosomes and remains part of the host cell genome for the rest of the cell's life. Non-limiting examples of retroviruses include lentiviruses such as HIV and SIV.

By “subject” is meant a mammal, such as a human patient or an animal (e.g., a rodent, bovine, equine, porcine, ovine, canine, feline, or other domestic mammal).

A “therapeutically effective amount” is an amount sufficient to effect a beneficial or desired clinical result. For example, a therapeutically effective amount is an amount sufficient to induce an immune response that prevents or treats an influenza infection.

By “treat” is meant to stabilize, reduce, or ameliorate the symptoms of any disease or disorder. It will be appreciated that, although not precluded, treating a disease or condition does not require that the disease, condition, or symptoms associated therewith be completely eliminated.

The term “vector” is meant to refer to the means by which a nucleic acid can be propagated and/or transferred between organisms, cells, or cellular components. Vectors include plasmids, viruses, bacteriophage, pro-viruses, phagemids, transposons, and artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not autonomously replicating.

The term “vector priming” is meant to refer to the delivery of a gene encoding a vaccine antigen by way of an expression vector. In embodiments, it means that the vector-based gene delivery will be a first exposure to the immunogenic composition, followed by one or more subsequent “booster” dose or doses of immunogenic composition.

“Viral load” is the amount of virus present in the blood of a patient. Viral load is also referred to as viral titer or viremia. Methods for evaluating viral load are well-known in the art.

Any compounds, compositions, or methods provided herein can be combined with one or more of any of the other compositions and methods provided herein.

### DESCRIPTION OF DRAWINGS

The abovementioned and other features and advantages of the present disclosure will be better understood when reading the following detailed description taken together with the following drawings in which:

FIG. 1 depicts a phylogenetic tree representing 2003 and 2005 HA subtypes. Human-Swine reassortant viruses and selected human influenza virus reference strains. Nucleotide sequences were aligned via Clustal W software and phylograms created by the Phylip program. The scale is number of nucleotide substitutions per 100.

FIG. 2 depicts an experimental timeline for testing vaccine dose response curves.

FIGS. 3A and 3B depict an exemplary NP encoding nucleotide sequence and polypeptide, respectively.

FIGS. 4A and 4B depict an exemplary variant 2 NP encoding nucleotide sequence and polypeptide, respectively.

FIGS. 5A and 5B depict an exemplary variant 3 NP encoding nucleotide sequence and polypeptide, respectively.

FIGS. 6A and 6B depict an exemplary PB1 encoding nucleotide sequence and polypeptide, respectively.

FIGS. 7A and 7B depict an exemplary variant 2 PB1 encoding nucleotide sequence and polypeptide, respectively.

FIGS. 8A and 8B depict an exemplary variant 3 PB1 encoding nucleotide sequence and polypeptide, respectively.

FIGS. 9A and 9B depict an exemplary PB2 encoding nucleotide sequence and polypeptide, respectively.

FIGS. 10A and 10B depict an exemplary variant 2 PB2 encoding nucleotide sequence and polypeptide, respectively.

FIGS. 11A and 11B depict an exemplary variant 3 PB2 encoding nucleotide sequence and polypeptide, respectively.

FIGS. 12A and 12B depict an exemplary PA encoding nucleotide sequence and polypeptide, respectively.

FIGS. 13A and 13B depict an exemplary variant 2 PA encoding nucleotide sequence and polypeptide, respectively.

FIGS. 14A and 14B depict an exemplary variant 3 PA encoding nucleotide sequence and polypeptide, respectively.

FIGS. 15A and 15B depict an exemplary NS2 encoding nucleotide sequence and polypeptide, respectively.

FIGS. 16A and 16B depict an exemplary variant 2 NS2 encoding nucleotide sequence and polypeptide, respectively.

FIGS. 17A and 17B depict an exemplary variant 3 NS2 encoding nucleotide sequence and polypeptide, respectively.

FIGS. 18A and 18B depict an exemplary M1 encoding nucleotide sequence and polypeptide, respectively.

FIGS. 19A and 19B depict an exemplary variant 2 M1 encoding nucleotide sequence and polypeptide, respectively.

FIGS. 20A and 20B depict an exemplary variant 3 M1 encoding nucleotide sequence and polypeptide, respectively.

FIGS. 21A and 21B depict an exemplary NS1 encoding nucleotide sequence and polypeptide, respectively.

FIGS. 22A and 22B depict an exemplary variant 2 NS1 encoding nucleotide sequence and polypeptide, respectively.

FIGS. 23A and 23B depict an exemplary variant 3 NS1 encoding nucleotide sequence and polypeptide, respectively.

FIG. 24 depicts an RNAg sequence corresponding to segment 1 of an exemplary influenza A genome.

FIG. 25 depicts an RNAg sequence corresponding to segment 2 of an exemplary influenza A genome.

FIG. 26 depicts an RNAg sequence corresponding to segment 3 of an exemplary influenza A genome.

FIG. 27 depicts an RNAg sequence corresponding to segment 4 of an exemplary influenza A genome.

FIG. 28 depicts an RNAg sequence corresponding to segment 5 of an exemplary influenza A genome.

FIG. 29 depicts an RNAg sequence corresponding to segment 6 of an exemplary influenza A genome.

FIG. 30 depicts an RNAg sequence corresponding to segment 7 of an exemplary influenza A genome.

FIG. 31 depicts an RNAg sequence corresponding to segment 8 of an exemplary influenza A genome.

FIGS. 32A and 32B depict a SDS-PAGE gel and Western Blot, respectively, analyzing NP protein expression.

FIGS. 33A and 33B depict a NP mass spectrometry peptide map, and an analysis of the resulting protein similarities, respectively.

FIG. 34 is a graph illustrating influenza A viral load in nasal swabs of NP vaccinated pigs.

FIGS. 35A and 35B depict a graph and a distribution plot, respectively, of the generation of antibodies against NP from NP vaccinated pigs.

FIG. 36 is a bar graph depicting the results of T-cell proliferation assays with respect to NP and M2E.

### DETAILED DESCRIPTION OF THE INVENTION

The present disclosure relates to compositions and methods for vaccinating humans and animals against enveloped viruses. More particularly, the present disclosure relates to compositions and methods for vaccinating humans and animals against influenza.

Eukaryotic viruses represent a large and diverse group of infectious agents known for the wide variety of diseases they cause. Viruses may be classified by a variety of criteria including genome structure (e.g. linear single strand or double strand DNA, circular double strand DNA, plus or minus strand RNA, or segmented RNA), capsid symmetry (e.g. helical, icosahedral, or complex), mode of replication (e.g. asymmetric, bidirectional/asymmetric, circularization/rolling circle, and concatemerization/asymmetric, reverse transcription, etc.), host specificity, and viral particle structure.

Based on their viral particle structure, viruses may be broadly classified as being either “non-enveloped” or “enveloped.” Non-enveloped viruses contain a viral genome that is surrounded by a proteinaceous capsid, which is formed by products encoded by the viral genome and synthesized by the machinery of the host cell. Enveloped viruses also have a viral genome surrounded by a proteinaceous capsid, however, the capsid is further encapsulated within an “envelope” comprised of a protein containing phospholipid bilayer. This envelope is acquired as the viral capsid buds through the cell membrane, and is usually derived from the outer membrane of the host cell, but may also be derived from the nuclear membrane, Golgi apparatus, or endoplasmic reticulum.

Enveloped viruses contain a variety of proteins within the phospholipid bilayer of their envelope that include both host cell encoded proteins (e.g. integral and trans-membrane proteins) and viral encoded proteins (e.g. glycoproteins). The composition of host cell encoded proteins found in the envelope of a particular enveloped virus varies with the cell type of the particular host cell that has been infected. The envelope serves an important function during viral replication by facilitating the entry of a virus particle into a new host cell. In this capacity, the viral glycoproteins within the envelope may function to recognize and bind specific molecules on the cell surface membrane of the target cell. For example, these molecules may include, but are not limited to, receptors, proteins, and lipids. Viral glycoprotein binding may then mediate fusion of the viral envelope with the target cell membrane and entry of the virus into the cell (e.g. by receptor-mediated endocytosis or direct fusion).

Enveloped viruses represent a diverse family of viruses that include, but are not limited to, arenaviridae, arteriviridae, asfarviridae, baculoviridae, bornaviridae, bunyaviridae, coronaviridae, filoviridae, flaviviridae, hepadnaviridae, herpesviridae, orthomyxoviridae, paramyxoviridae, poxyiridae, retroviridae, rhabdoviridae, and togaviridae. These virus families are responsible for a wide variety of human and animal diseases including, but not limited to, encephalitis, gastro-intestinal disease, hemorrhagic disease, hepatitis, immunosuppressive diseases, ocular disease, pox (e.g. chickenpox, cowpox, smallpox, monkeypox, felinepox, swinepox, and pseudo-cowpox), respiratory disease, sexually transmitted disease, and cancer, and result in billions of infections, and millions of deaths, world wide every year. For example, infections with influenza viruses in humans are a common and significant cause of respiratory disease and result in an average of approximately 20,000 deaths and 114,000 hospitalizations per year in the U.S. alone. Severe outbreaks of influenza periodically affect millions of people. In one instance, the 1918-1919 Spanish flu pandemic was responsible for killing an estimated 20 million people. In another instance, 80% of the U.S. Army's casualties during World War I were attributed to influenza.

Influenza belongs to the orthomyxoviridae family of viruses, which encompasses several genera including influenza A, influenza B, and influenza C. Of these, the most virulent human pathogen is influenza A, which is a negative stranded, segmented, enveloped RNA virus comprised of a helical ribonucleocapsid known as the viral ribonucleoprotein (vRNP). The vRNP contains a genome comprised of eight negative sense RNA segments that encode eleven viral proteins, including: hemagglutinin (HA), neuraminidase (NA), nucleoprotein (NP), M 1, M2, NS1, NS2 (aka NEP), PA, PB1, PB1-F2, and PB2. The genome segments are packed in a helical form with NP, and the resulting RNP structures are associated with the three individual subunits (PB 1, PB2, and PA) of the viral polymerase, which is known as the 3P complex in its assembled form. The vRNP may also be associated with NS2. Viral particles are packaged when RNPs accumulate at the cell surface near regions of the host cell membrane that have accumulated the viral transmembrane envelope proteins, HA, NA, and M2. The virus particle acquires its envelope as it buds from the host cell. HA trimers and NA tetramers display prominent glycoprotein spikes on the exterior surface of the envelope, with HA being the predominant envelope protein. The M2 protein encodes an ion channel, and is present in the envelope at a very low concentration estimated to be 16-20 molecules per virion. The viral protein M1 is located on the interior surface of the viral envelope, and is believed to play a role in linking the internal domains of HA and NA with the RNP.

The morphology of influenza viral particles is highly variable, ranging from discrete spherical particles to filamentous particles that can be up to 2000 nm long. The most striking feature of influenza virions is the dense layer of HA and NA spikes projecting radially outward over the surface of the viral envelope, and the epitopes encoded by these spikes represent important targets for the immune system of the host (e.g. animal or human).

HA and NA play essential roles in the viral replication process. HA is believed to facilitate virus binding by interacting with sialic sugars present on all glycoproteins and glycolipids on the cell surface of target epithelial cells. Once bound, viral particles enter the cell by endocytosis. Enzymatic or proteolytic cleavage of the HA protein is essential for viral infection, as cleaved HA allows the endocytosed particle to fuse with the endosomal membrane. The cleavage of HA is modulated by the number of basic amino acid residues located at the cleavage site, and this composition is important in determining overall virulence. NA is important during the shedding process, as this protein cleaves sialic acid residues, thereby facilitating the release of the viral particle.

Influenza viruses periodically undergo significant antigenic variation as a result of the processes of antigenic drift and antigenic shift. Antigenic “drift” involves minor antigenic changes in the envelope proteins hemagglutinin (HA) and neuraminidase (NA), while antigenic “shift” involves major antigenic changes in these molecules. Changes in the conformation of these two antigens are accompanied by changes in antigenicity, which facilitates the ability of the viral particle to evade detection by the immune system. The genomic changes associated with antigenic drift are facilitated by the fact that the 3P polymerase complex has no proofreading activity; consequently, the polymerase introduces errors into the genome during viral replication at a rate of approximately one base change error per genome per replication cycle. This represents a substantial rate of mutation. Furthermore, antigenic shift is facilitated by the division of the RNA genome into multiple segments because the infection of a single cell by multiple strains of influenza virus may lead to reassortment of RNA segments from multiple strains into a single viral particle, effectively “shuffling” the viral genome. The combination of antigenic drift and shift allows the genetic constitution of influenza particles to change very rapidly.

These changes in genetic composition are tracked within influenza virus nomenclature, which categorizes strains by their origin and antigenic constitution of their HA and NA loci. For example, a swine isolate from Iowa may be categorized A/swine/Iowa (H1N1), where the H and N refer to the HA and NA subtypes, respectively. Extent influenza subtypes include, but are not limited to, fifteen HA subtypes (H1-H15) and nine NA subtypes (N1-N9) that have been identified to date. Characterized influenza A subtypes include, but are not limited to, H1N1 (1918 Spanish flu pandemic), H2N2 (1957 Asian flu pandemic), H3N2 (1968 Hong Kong flu pandemic), H5N1 (current pandemic threat), H7N7 (current zoonotic threat), H1N2 (endemic in humans and swine), H9N2, H7N2, H7N3, and H10N7.

In the United States vaccines are prepared each year for the annual vaccination program, with the quantity of immunoreactive HA in each dose standardized to contain the amount recommended by the Bureau of Biologics, U.S. Food and Drug Administration (FDA). The resistance to infection after vaccine administration correlates with the level of serum antibody produced to the specific strain active that year. In other words, antigenic changes within the HA and NA subtypes described above require concomitant changes in the vaccine, according to the type(s) of antigen present.

Influenza A viruses have been isolated from a number of animal hosts including humans, birds, and pigs. Pandemic influenza in humans can be a zoonotic disease caused by transfer of influenza A viral genome segments from animal reservoirs. In recent years the prevalence of swine influenza virus (SI) has increased in U.S. swine populations and several new subtypes have been identified. Pigs are postulated to serve as the “missing vessel” hosts in which reassortment between avian and human viruses can generate genetically novel viruses with pandemic potential. Additionally, the likelihood of a human pandemic has increased dramatically with the natural transmission of the highly pathogenic avian influenza virus (H5N1) directly to humans.

SIV is an important etiological agent involved both in epizootic and enzootic forms of influenza. The swine disease is an important model for human influenza with similar clinical symptoms and pathogenesis that results in nearly 100% morbidity. Clinical signs of the epizootic form are a deep-dry, “barking” cough, fever above 42° C. and anorexia. Sows infected during pregnancy may abort as a result of the high fever. Clinical signs of the enzootic form are coughing, fever, anorexia, and poor performance. Human-like H1N1 and H1N2 reassortant SIVs have been isolated from pigs, and are associated with high morbidity.

When exposed to SIV under natural conditions, pigs develop antibodies to HA epitopes on the viral envelope via the humoral immune response. This humoral immunity appears to be subtype- and strain-specific, meaning H1N1-induced antibodies will not confer protection against an H3N2 infection and vice versa. In addition to the humoral response, natural exposure to SIV also generates anti-SIV antibodies via the cell-mediated immune (CMI) response. Interestingly, preliminary research in pigs suggests that the CMI response is directed primarily against non-envelope proteins, for example, NP and M1. In contrast, vaccination against SIV generates a markedly different response in pigs. As with natural exposure to the virus, vaccination induces a humoral immune response to the HA subtype of the vaccine strain; however, in contrast to the case with natural exposure, vaccination does not induce an appreciable CMI response. Furthermore, protection provided by vaccination is incomplete in the face of heterologous challenge, allowing infection and replication of virus, albeit at reduced levels. The observation that the CMI response appears to preferentially target non-envelope proteins is significant because viral nucleoproteins (including NP of influenza A) are often highly conserved between strains, unlike their protein counterparts within the viral envelope.

There are few examples in the art of the use of non-envelope protein compositions as the basis for viral vaccines and therapeutics. Dietzschold et al. (1987) attempted to use purified vRNP from the rabies virus (an enveloped virus belonging to the Rhabdoviridae family) as a vaccine to generate an immuno-protective response. Disadvantageously, this approach failed to protect mice against a viral challenge when injected intra-cerebrally, whereas a purified rabies envelope protein (G protein) provided effective immuno-protection against a live virus challenge when administered intra-cerebrally. Dietzschold et al. (1987) showed that vRNP from rabies could generate an immuno-protective response when administered intra-peritoneally. Unfortunately, intra-peritoneal administration of purified rabies vRNP was only capable of generating a homologous and/or heterologous protective response in approximately 80% of the mouse and raccoon populations tested, an unacceptably low efficacy rate. A further disadvantage of this work is that the vRNP purification protocol used by Dietzschold et al (1987) does not preclude the presence of contaminating envelope proteins. Even at undetectable levels, such proteins could exert immunogenic responses in test subjects that would affect the interpretation of the observed results. These authors also attempted to use two different peptide fragments of the vRNP associated protein N as vaccines. Disadvantageously, one of these peptides failed to confer any immuno-protection, while the other conferred protection that was highly variable within the experimental population, ranging between 62-82%.

Ertl et al. (1989) tested a panel of 40 overlapping peptides derived from the N protein of rabies for efficacy as a vaccine, and found that some of these peptides were able to augment the production of rabies neutralizing antibodies. Unfortunately, none of the 40 peptides tested were able to immunize mice against a rabies viral challenge.

Sumner et al. (1991) demonstrated that the use of recombinant N protein (fusion with vaccinia TX protein) from rabies was able to immunize mice against a viral challenge with street rabies. A problem with this approach is that this immuno-protective response required inoculation with recombinant N protein at a much higher concentration (107 PFU vs. 103 PFU) then that required for inoculation with the rabies envelope protein G. A further disadvantage of these studies is that they rely on the use of a fusion protein.

Wraith et al. (1987) attempted to use purified NP protein to immunize mice against the influenza virus. Disadvantageously, these authors found that the use of purified NP protein protected only 75% of the test subjects from subsequent challenge by the influenza virus (Wraith et al. 1987). A further disadvantage of these studies arises from the fact that the authors acknowledge that their ‘purified’ NP protein samples may contain up to 3% contamination with the envelope protein HA.

According to an illustrative embodiment, compositions of the current disclosure comprise naked viral core particles (NVCPs) that may be used as vaccines to generate an immuno-protective response in humans and animals, or as therapeutics to reduce viral shedding and improve post-challenge clinical outcome of infected patients or animals. NVCPs may be comprised of the viral genome (RNAg), as well as various genome associated proteins, but lack the viral envelope and are avirulent as a result. For example, an influenza NVCP may be comprised of, but is not limited to, RNAg, NP, PB1, PB2, PA, and NS2.

NVCPs may be prepared by using an animal or cell culture system to produce virus that may be isolated and subjected to standard biochemical and molecular biological techniques to remove the envelope and envelope associated proteins. For example, influenza virus may be produced using cell culture systems such as, but not limited to, the ones described in U.S. Pat. No. 4,500,513 and U.S. Publication No. 2008/0274141, hereby incorporated by reference in their entirety. Once the influenza virus has been produced and purified, influenza NVCPs may be isolated using standard biochemical purification protocols (see e.g., Schneider et al, 1973). One of ordinary skill in the art will recognize that the NVCP isolation protocols may vary substantially depending on the type of enveloped virus being purified.

It is also contemplated within the scope of the invention that NVCPs may be synthesized de novo. For example, the ssRNA genome segments may be synthesized using vectors that allow ssRNA production and purification, while the genes encoding the genome associated proteins may be subcloned into suitable expression vector systems. The resulting RNA segments and expressed proteins may then be combined to produce synthetic NVCPs.

Internal moieties associated with NVCPs generally display high levels of amino acid conservation across strains, relative to the highly variable moieties associated with envelope proteins. Advantageously, this high level of internal moiety conservation means that NVCPs may provide immuno-protection that is strain independent (i.e. both homologous and heterologous protection). A further advantage of NVCPs is that they may provide immuno-protection without compromising the efficacy of immuno-detection assays used to detect viral infection, which are generally based on serotypes generated against proteins within the viral envelope.

Without being bound by any particular theory, it is believed that NVCPs may induce immuno-protection by multiple, non-exclusive, mechanisms. One mechanism may be the direct production of antibodies against the nucleoprotein components via the humoral response. Another mechanism may be the production of antibodies against the nucleoprotein components by the CMI response. Yet another mechanism may be that NVCPs provide immuno-protection as a result of a direct induction of T-helper cells that augment the activity of virus neutralizing antibody producing B cells. Protection induced in this manner may be attributed to the priming of influenza nucleoprotein by specific cytolytic T cells, which represents a direct activation of cellular immunity by a purified, non-enveloped portion of the virus. Yet another possible immuno-protective mechanism may comprise the direct targeting of the viral genome by siRNA or RNAi mediated pathways by the ssRNA components of the NVCP.

It is contemplated within the scope of the disclosure that compositions for vaccinating humans and animals against influenza may further comprise virus-like particles (VLPs) that consist of the viral genome in combination with any one of the genome associated proteins. For example, such VLP compositions may be comprised of RNAg and NP, RNAg and PB1, RNAg and PB2, RNAg and PA, or RNAg and NS2.

It is further contemplated within the scope of the disclosure that such VLP compositions may also be comprised of the viral genome in combination with any two genome associated proteins. For example, such VLP compositions may include, but are not limited to, RNAg and NP plus PB1, or RNAg and NP plus PB2, or RNAg and NP plus PA, or RNAg and NP plus NS2. It is still further contemplated that such VLP compositions may also be comprised of the viral genome in combination with any three, or any four, of the genome associated proteins.

The invention also provides constructs comprising a nucleic acid molecule and methods for producing a VLP comprising influenza polypeptides, or fragments thereof, in a nonpermissive cell type, as well as compositions and methods that increase the efficiency of VLP production in such cells. In various embodiments, the nucleic acid molecules are useful for in vitro or in vivo expression (i.e., expression in a human or porcine subject having, or at risk of developing, an influenza infection). For example, the use of a cRNA promoter, or portions thereof, in an expression vector comprising a nucleic acid molecule of the invention can improve the efficiency of influenza protein production in a cell. In another example, a 3′ UTR is included in the expression vector. A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

Constructs and/or vectors provided herein comprise influenza polynucleotides that encode structural polypeptides, or portions thereof as described herein. The vector may be, for example, a phage, plasmid, viral, or retroviral vector. The constructs and/or vectors that comprise the nucleotides should be operatively linked to an appropriate promoter, such as the cRNA influenza promoter, CMV promoter, phage lambda PL promoter, the E. coli lac, phoA and tac promoters, the SV40 early and late promoters, and promoters of retroviral LTRs are non-limiting examples. In one embodiment, the promoter is an influenza cRNA promoter. Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the polypeptide to be translated. If desired, the vector further comprises a 3′ UTR, such as an influenza 3′ UTR.

Expression vectors will typically include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBac1 pWINEO, pSV2CAT, pOG44, pXT1 and pSG, pSVK3, pBPV, pMSG, and pSVL. In particular embodiments, the vector is a bicistronic vector (e.g., pIRES). Other suitable vectors will be readily apparent to the skilled artisan.

Recombinant constructs can be prepared and used to transfect, infect, or transform and can express viral proteins, including those described herein, into eukaryotic cells and/or prokaryotic cells. Thus, the invention provides for host cells which comprise a vector (or vectors) that contain nucleic acids which code for influenza proteins in a host cell under conditions which allow the formation of VLPs.

In another embodiment, the vector and/or host cell comprise nucleotides that encode influenza proteins, or portions thereof as described herein. In another embodiment, the vector encodes a protein that consists essentially of influenza NP, PB1, PB2, PA, NS1, NS2, and M1, or portions thereof as described herein.

Once a recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

Methods to grow cells that produce VLPs of the invention include, but are not limited to, batch, batch-fed, continuous and perfusion cell culture techniques. In one embodiment, a cell comprising an influenza nucleic acid molecule is grown in a bioreactor or fermentation chamber where cells propagate and express protein (e.g. recombinant proteins) for purification and isolation. Typically, cell culture is performed under sterile, controlled temperature and atmospheric conditions. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In other embodiment, the pre-sterilized plastic bags are about 50 L to 1000 L bags.

The VLPs are isolated using methods that preserve the integrity thereof, such as by gradient centrifugation, e.g., cesium chloride, sucrose and iodixanol, as well as standard purification techniques including, e.g., ion exchange and gel filtration chromatography. The following disclosure is an example of how VLPs of the invention can be made, isolated and purified. A person of skill in the art appreciates that there are additional methods that can be used to make and purify VLPs. Accordingly, the invention is not limited to the methods described herein.

The invention also provides for a pharmaceutical pack or kit comprising one or more containers filled with one or more of the ingredients of the polynucleotide and or VLP vaccine formulations of the invention. In a preferred embodiment, the kit comprises two or more containers, one containing VLPs, another containing a nucleic acid molecule and, optionally, another containing an adjuvant. Associated with such container(s) can be a notice in the form prescribed by a governmental agency regulating the manufacture, use or sale of pharmaceuticals or biological products, which notice reflects approval by the agency of manufacture, use or sale for human administration.

The invention also provides that the nucleic acid molecules and/or VLP formulations be packaged in a hermetically sealed container such as an ampoule or sachette indicating the quantity of composition. In one embodiment, the nucleic acid molecule and/or VLP composition is supplied as a liquid, in another embodiment, as a dry sterilized lyophilized powder or water free concentrate in a hermetically sealed container and can be reconstituted, e.g., with water or saline to the appropriate concentration for administration to a subject.

The invention also features a kit comprising a nucleic acid molecule and/or VLP as described herein and instructions for use in an immunization method delineated herein.

In another embodiment, compositions of the disclosure may be comprised of the RNAg, in the absence of any of the internal viral proteins. It is further contemplated that RNAg-derivative compositions may comprise subsets of the viral genome. For example, an RNAg-derivative composition for influenza may comprise any combination of 1, 2, 3, 4, 5, 6, or 7 of the negative sense RNA segments of the RNAg.

In another embodiment, compositions of the disclosure may be comprised of any combination of 1, 2, 3, 4, or 5 of the internal viral proteins (IVPs)—NP, PB1, PB2, PA, and NS2—in the absence of the RNAg.

In yet another embodiment, compositions of the disclosure may be comprised of derivative internal viral proteins (DIVPs), either individually or in combination. Such DIVPs may include C-terminal, N-terminal, or internal deletions of NP, PB 1, PB2, PA, and/or NS2. It is also contemplated that such DIVPs may include peptides corresponding to discrete regions within NP, PB 1, PB2, PA, and/or NS2.

In another embodiment, compositions of the disclosure may be comprised of recombinant fusion proteins (RFPs). Such RFPs may include either N-terminal or C-terminal fusions of NP, PB 1, PB2, PA, and NS2 with proteins or protein fragments that may increase the immunogenicity of the resulting recombinant protein. For example, candidate proteins or protein fragments for use with influenza specific compositions may include, or be derived from, HA, NA, or M2.

It is contemplated within the scope of the invention that such IVPs, DIVPs, and/or RFPs may be synthesized by a variety of standard molecular biology methods. For example, the entire open reading frame of SIV H1N1 (A/Swine/North Carolina/38448-1/2005) may be amplified and cloned into the Gateway™ (Invitrogen, Carlsbad Calif.) baculovirus cloning system per manufacturer's recommendations. The Gateway® Technology is based on the bacteriophage lambda site-specific recombination system which facilitates the integration of lambda into the E. coli chromosome and the switch between the lytic and lysogenic pathways (Ptashne, 1992). In the Gateway® Technology, the components of the lambda recombination system are modified to improve the specificity and efficiency of the system (Bushman et al., 1985). In brief, an IVP gene may be cloned by lambda recombination into a baculodirect linear DNA vector and transformed into cells. Recombinant clones may be selected using ganciclovir, and the P1 virus will be harvested and used to transfect Sf-9 cells. The viral DNA may be extracted and the insert sequence confirmed by direct nucleotide sequencing. Transfected cells may be screened for recombinant protein production by Western analysis. Positive recombinant clones may then be maintained in insect cells. Baculovirus titers may be determined and the recombinant proteins (as IVPs) will be purified using standard protocols. The purity of the recombinant proteins may be determined using standard SDS-PAGE and Western blot analyses. Purified protein may also be analyzed by immune electron microscopy to confirm IVP assembly and for amino acid sequence by standard mass spectrometry analysis.

More specifically, Sf-9 cells may be prepared as spinner cultures using about 0.5 to 1×10⁶ cells/ml in about 300 mls total volume with media fortified with about 1% Pluronic F-68 (JRH) to prevent cell clumps, about 10% FBS, and about 1% antibiotics. The cells may be seeded in flasks when cell density approaches about 2×10⁶ cells/ml but does not exceed about 3×10⁶ cells/ml. The cells should be sub-cultured every other day.

High-Five cells may be prepared as spinner cultures using about 0.5×10⁶ cells/ml in about 300 mls total volume. The media may be fortified with about 1% Pluronic F-68 (JRH) to prevent cell clumps, about 10% FBS and about 1% antibiotics. The cells may be seeded in flasks when the cell density approaches about 1.5×10⁶ cells/ml. Generally, H-5 cells will not grow to higher density than about 2×10⁶ cells/ml. These cells should be sub-cultured daily.

Stock virus may be prepared by seeding about 1.5 to 2×10⁷ cells in 8-10 ml volume in T-162 flasks. The cells may be allowed to attach for about 30 minutes, after which time the media may be removed and baculovirus added at an MOI of about 0.01. Media supplemented with about 10% FBS may then be added, followed by virus harvesting at about 10 days post-infection. The stock virus may be titrated, aliquoted, and stored at about −80° C.

For IVP production, about 3×10⁷ cells will be seeded in about 8-10 ml volume in T-162 flasks. The cells may be allowed to attach for about 30 minutes. For H-5, about 2×10⁷ cells may be used. The media may be removed followed by the addition of recombinant baculoviruses RFVP2, VP6, VP4, and VP7 at MOI of about 5 each, diluted to a final volume of about 4 ml. The viruses may be attached to cells by rocking the flasks at low speed for about 3 hours. Media supplemented with about 1% FBS (final FBS should be about 2%—taking into account that stock viruses are in about 10% FBS) may be added, and virus harvested after about 7 days post infection.

For purification of IVPs, the supernatant may be centrifuged at about 2,000×g for about 30 minutes. The supernatant may be underlain with about 35% sucrose in TNC buffer followed by centrifugation at about 25,000 rpm for about 2 hours. The pellet from about 180-240 ml supernatant may be resuspended in about 4 ml of TNC buffer followed by the addition of about 1.89 g CsCl₂. Since H-5 cells will have higher yield, the same volume of supernatant in H-5 may need to be resuspended in about 8 ml of TNC buffer. After centrifugation at about 35,000 rpm for about 18 hours, the bands may be collected and washed with TNC buffer at about 25,000 rpm for about 1.5 hours. The pellet may be resuspended in about 1 ml of TNC buffer. The pellet may be stored at about 4° C. prior to analysis by ELISA, immune electron microscopy, and western blot.

It is also contemplated that peptides of the present disclosure described above, particularly DIVPs, may be chemically synthesized. Such synthetic polypeptides may be prepared using standard techniques of solid phase, liquid phase, or peptide condensation techniques, or any combination thereof, and may include natural and/or unnatural amino acids. Amino acids used for peptide synthesis may be standard Boc (N^{a}-amino protected N^{a}-t-butyloxycarboyl) amino acid resin with the standard deprotecting, neutralization, coupling and wash protocols of the original solid phase procedure of Merrifield [J. Am. Chem. Soc., 85:2149-2154 (1963)], or the base-labile N^{a}-amino protected 9-fluorenylmethoxycarbonyl (Fmoc) amino acids first described by Carpino and Han [J. Ovg. Chem., 37:3403-3409 (1972)]. Both Fmoc and Boc N^{a}-amino protected amino acids may be obtained from Advanced Chemtech, Cambridge Research Biochemical, Fluka, Sigma or other chemical companies familiar to those of skill in the art. In addition, the method of the invention may be used with other N^{a}-protecting groups familiar to those skilled in the art. Solid phase peptide synthesis may be accomplished by standard techniques including, but not limited to, those described in Stewart and Young, 1984, Solid Phase Synthesis, Second Edition, Pierce Chemical Co., Rockford, Ill.; Fields et al., Znt. J. Pept. Protein. 35:161-214 (1990), or by using automated synthesizers, such as those sold by ABI. Thus, polypeptides of the disclosure may comprise D-amino acids, a combination of D- and L-amino acids, and various “designer” amino acids (e.g., -methyl amino acids, Ca-methyl amino acids, and N^{a}-methyl amino acids, etc.) to convey special properties. Synthetic amino acids may include ornithine for lysine, fluoro-phenylalanine for phenylalanine, and norleucine for leucine or isoleucine. Additionally, by assigning specific amino acids at specific coupling steps, α-helices, β-turns, β-sheets, and cyclic peptides may be generated, further increasing the potential immunogenicity of such DIVP peptides.

One of ordinary skill in the art may employ conventional biochemistry, molecular biology, microbiology, immunology, and recombinant DNA techniques to prepare the compositions of the current disclosure. Explanations of such techniques may be found in standard laboratory reference materials including, but not limited to, the following: Molecular Cloning: A Laboratory Manual, Sambrook et al, (3rd edition, 2001); Current Protocols in Molecular Biology, Volumes I-III, Ausubel, R. M., ed., (1999 plus subsequent updates); Cell Biology: A Laboratory Handbook, Volumes I-III, J. E. Celis, ed., (1994); Current Protocols in Immunology, Volumes I-IV, Coligan, J. E., ed. (1999 plus subsequent updates); Oligonucleotide Synthesis, M. J. Gait ed., (1984); Nucleic Acid Hybridization, B. D. Hames & S. J. Higgins eds., (1985); Transcription And Translation, B. D. Hames & S. J. Higgins, eds., (1984); Culture of Animal Cells (4th edition), R. I. Freshney, ed., (2000); Immobilized Cells And Enzymes, IRL Press, (1986); A Practical Guide To Molecular Cloning, B. Perbal, (1988); Using Antibodies: A Laboratory Manual, Harlow, E. and Lane, D. (Cold Spring Harbor Press, 1999), which are hereby incorporated by reference in their entirety.

The above described compositions of the disclosure preferably further comprise an adjuvant. For example, such adjuvants may include, but are not limited to, aluminum phosphate, aluminum hydroxide, Squalene, QS21, MF59 (Chiron), and complete Freund's adjuvant. Adjuvant properties may also be effected by the use of virosomes to deliver compositions of the disclosure. Many other adjuvants for use in vaccines are known in the art, and may be readily adapted for use with the compositions of the current disclosure by one of ordinary skill in the art. See, for example, Vaccine Adjuvants and Delivery Systems, Singh, M. ed., (2007); Vaccine Adjuvants: Immunological and Clinical Principals, Hackett, C. J. and Ham, D. A. ed., (2006); and Vaccine Adjuvants: Preparation Methods and Research Protocols, O'Hagan, D. T. ed., (2000). The vaccine may be prepared using any pharmaceutically acceptable carrier or vehicle, including, water, Hanks basic salt solution (HBSS) or phosphate buffered saline (PBS), with or without a preservative. The vaccine may be lyophilized for resuspension at the time of administration. Vaccines may be prepared for use in both active and passive immunizations. In the case of DIVP compositions, the antigenic material is preferably extensively dialyzed to remove undesired small molecular weight molecules. If desired, it may be lyophilized for more ready formulation into a desired vehicle.

The preparation of vaccines that contain peptide sequences as active ingredients is generally well understood in the art, as exemplified by U.S. Pat. Nos. 4,608,251; 4,601,903; 4,599,231; 4,599,230; 4,596,792; and 4,578,770 (all incorporated herein by reference in their entirety). Generally, such vaccines are prepared as injectables, either as liquid solutions or suspensions. Solid forms suitable for dissolution, or suspension, in liquid prior to injection may also be prepared. Such preparations may also be emulsified. The active immunogenic ingredient may be mixed with pharmaceutically acceptable excipients that are compatible with the NVCP, VLP, IVP, DIVP, and/or RFP containing compositions. Suitable excipients may include, but are not limited to, water, saline, dextrose, glycerol, ethanol, or the like, and combinations thereof. Additional examples of substances that may serve as pharmaceutically acceptably carriers are sugars (e.g. lactose, glucose, sucrose, and the like), starches (e.g. corn starch, potato starch, and the like), cellulose and its derivatives (e.g. sodium carboxymethylcellulose, ethylcellulose, cellulose acetate, and the like), powdered tragacanth, malt, gelatin, talc, stearic acid, magnesium stearate, calcium sulfate, vegetable oils (e.g. peanut oil, cottonseed oil, sesame oil, olive oil, corn oil, oil of theobroma, and the like); polyols (e.g. propylene glycol, glycerine, sorbitol, mannitol, polyethylene glycol, and the like), agar, alginic acid, pyrogen-free water, isotonic saline, and phosphate buffer solutions, as well as other non-toxic compatible substances used in pharmaceutical formulations. In addition, the vaccine may contain minor amounts of auxiliary substances such as wetting or emulsifying agents, pH buffering agents, or the above described adjuvants that enhance the efficacy of the vaccines.

Suitable methods of vaccine administration include, but are not limited to, topical (e.g. epidermal abrasion), oral, anal, vaginal, intravenous, intraperitoneal, intramuscular, subcutaneous, nasopharyngeal, and intradermal administration. Preferred routes of administration include intraperitoneal, intramuscular, subcutaneous, and intradermal. The most preferred route of administration is intradermal. In a preferred embodiment, a vaccine is packaged in a single dosage for immunization by intramuscular, intradermal, intraperitoneal, or nasopharyngeal administration. In the most preferred embodiment it is packaged in a single dosage for intradermal administration.

It will be recognized by those of skill in the art that an optimal dosing schedule of a vaccination regimen will vary according to the particular composition being used. For example, a dosing schedule may include as many as about five to six, but preferably about three to five, or even more preferably about one to three administrations of the immunizing composition. Such administrations may be given at intervals of as short as about two to four weeks, or as long as about five to ten years. Such administration may occasionally be given at even longer intervals.

In yet another embodiment of the disclosure, a pharmaceutical composition is provided that comprises the NVCP, VLP, IVP, DIVP, and/or RFP containing compositions described above optionally in combination with pharmaceutically acceptable excipients, in a therapeutically effective amount to treat influenza infection.

Methods for preparing pharmaceutical compositions that contain polypeptides or peptide fragments as active ingredients are known in the art. Typically, such compositions are prepared as injectables, either as liquid solutions or suspensions, however, solid forms suitable for dissolution, or suspension, in liquid prior to injection may also be prepared. The preparation may also be emulsified, or encapsulated within liposome complexes. The active therapeutic ingredient is often mixed with excipients which are pharmaceutically acceptable and compatible with the active ingredient. Suitable excipients are, for example, water, saline, dextrose, glycerol, ethanol, or the like and combinations thereof. If desired, the composition may also contain minor amounts of auxiliary substances such as wetting agents, emulsifying agents, or pH buffering agents that enhance the effectiveness of the active ingredient.

A polypeptide or peptide fragment may be formulated into the therapeutic composition as neutralized pharmaceutically acceptable salt forms. Pharmaceutically acceptable salts include the acid addition salts (formed with the free amino groups of the polypeptide or antibody molecule) that are formed with inorganic acids (e.g., hydrochloric acid, phosphoric acids, or the like) or organic acids (e.g., acetic, oxalic, tartaric, mandelic, and the like). Salts formed from the free carboxyl groups may also be derived from inorganic bases (e.g., sodium, potassium, ammonium, calcium, ferric hydroxides, or the like) and/or organic bases (e.g., isopropylamine, trimethylamine, 2-ethylamino ethanol, histidine, procaine, and the like).

The therapeutic polypeptide or peptide fragment containing compositions are conventionally administered intravenously, for example by injection of a unit dose, which refers to physically discrete units suitable as unitary dosage for humans or animals, each unit containing a predetermined quantity of active material calculated to produce the desired therapeutic effect in association with the required diluent.

Potential enveloped virus targets for these vaccination methods may be identified in the following virus families that include, but are not limited to, arenaviridae, arteriviridae, asfarviridae, baculoviridae, bornaviridae, bunyaviridae, coronaviridae, filoviridae, flaviviridae, hepadnaviridae, herpesviridae, orthomyxoviridae, paramyxoviridae, poxyiridae, retroviridae, rhabdoviridae, and togaviridae. More preferred enveloped virus targets for these vaccination methods may be identified in the following virus families, herpesviridae, orthomyxoviridae, poxyiridae, and retroviridae. The most preferred enveloped virus targets for these vaccination methods may be identified in the orthomyxoviridae and retroviridae virus families. In particular, preferred viral targets for vaccination may include influenza (orthomyxoviridae) and HIV (retroviridae).

The above detailed description presents one illustrative embodiment of the current disclosure in the form of compositions and methods for vaccinating humans and animals against the influenza virus. For the sake of clarity, terminology is used in this detailed description that is specific for this particular embodiment of the disclosure; however, this terminology is not intended to be limiting, and should not be construed as limiting insofar as one skilled in the art will recognize that many different forms and variations of the current disclosure will be generally applicable to all enveloped viruses, and are possible within the scope of the appended claims. All references cited in this disclosure are hereby incorporated by reference in their entirety.

### Example 1

Studies at the University of Minnesota are evaluating protective immunity induced by vaccination with a commercially available, inactivated H1N1 and H3N2 bivalent SIV against challenge with recent field isolates of H1N1 or H3N2 swine influenza viruses (Gramer and Rossow, 2004; Lee, 2007). In both studies, pigs are vaccinated twice per label directions and then challenged intranasally with about 1 ml of about 10⁶ TCID50/ml per nostril of heterologous H1N1 virus (NSwine/MN/00040/2002) in trial 1 or with about 1 ml of about 10⁶ TCID50/ml per nostril of heterologous H3N2 virus (A/Swine/Colorado/00294/2004) in trial 2. In both trials, pigs are evaluated daily post challenge for clinical signs of respiratory disease or fever and nasal swabs are collected for virus detection. About five to seven days post-challenge, pigs are euthanized and necropsied for evaluation of pneumonia. Clinical signs and pneumonia lesions are reduced in the vaccinated groups when compared to the unvaccinated groups. However, virus is still detectable in nasal swabs taken from some vaccinated pigs.

### Example 2

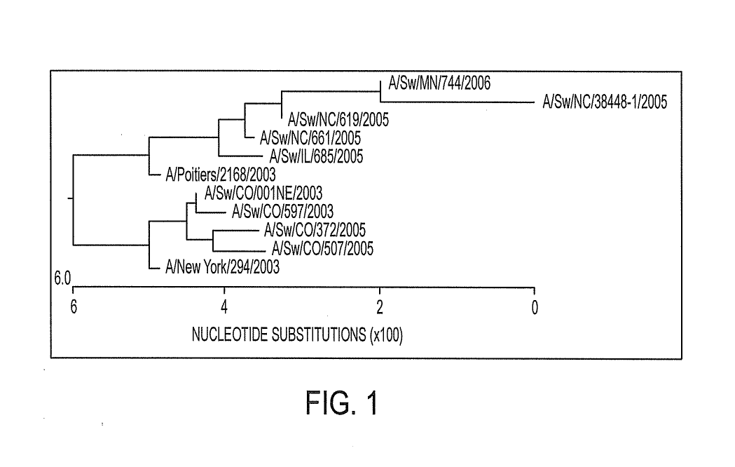

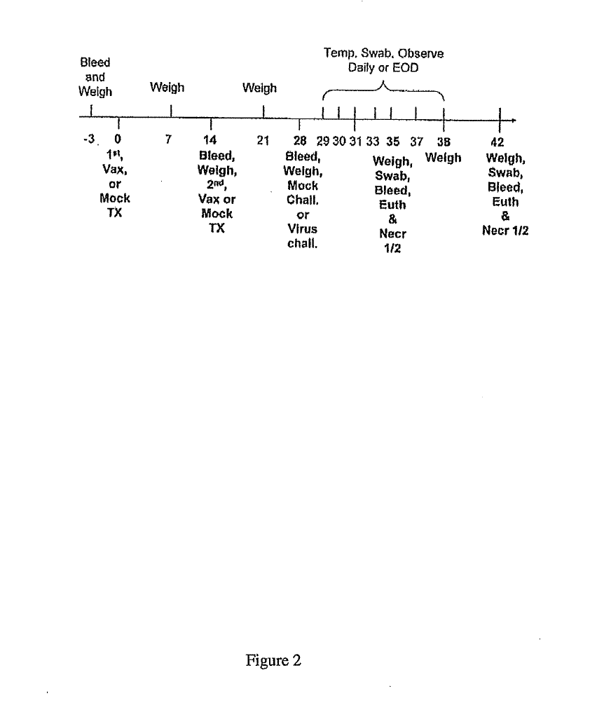

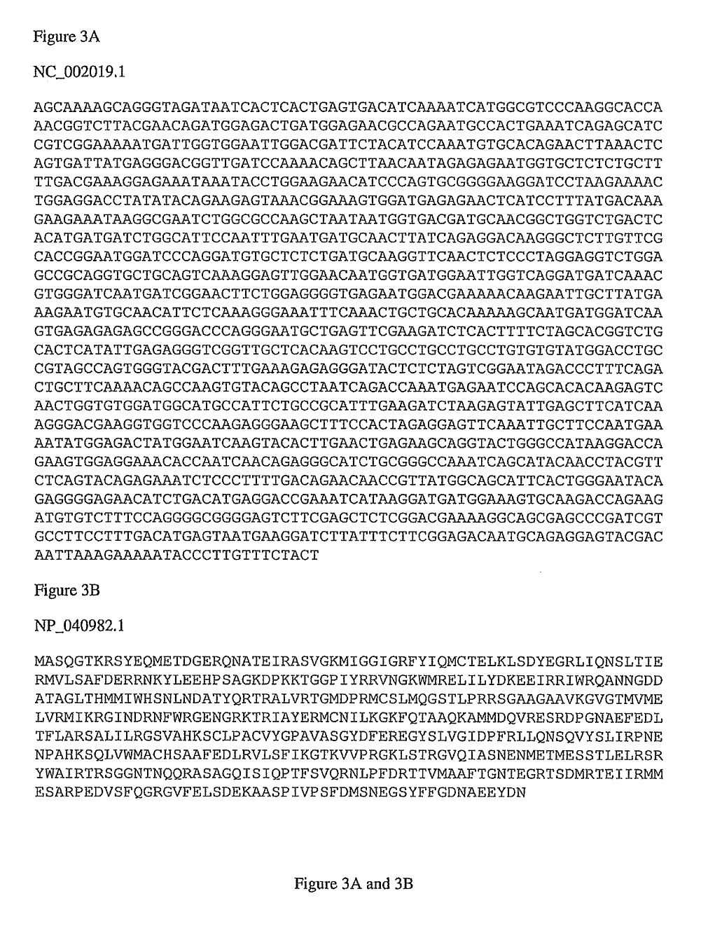

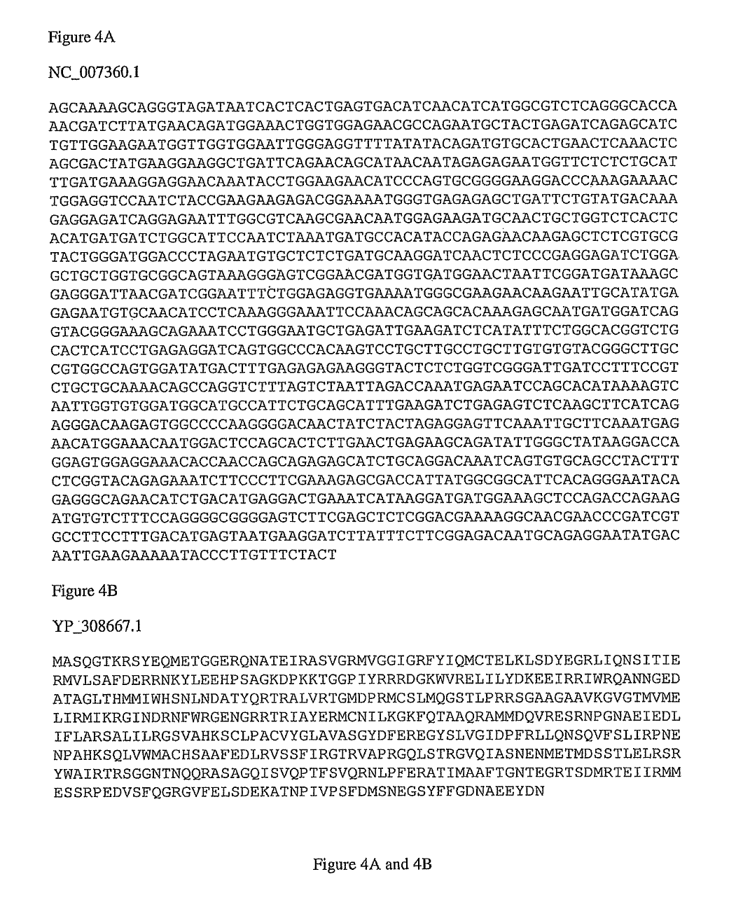

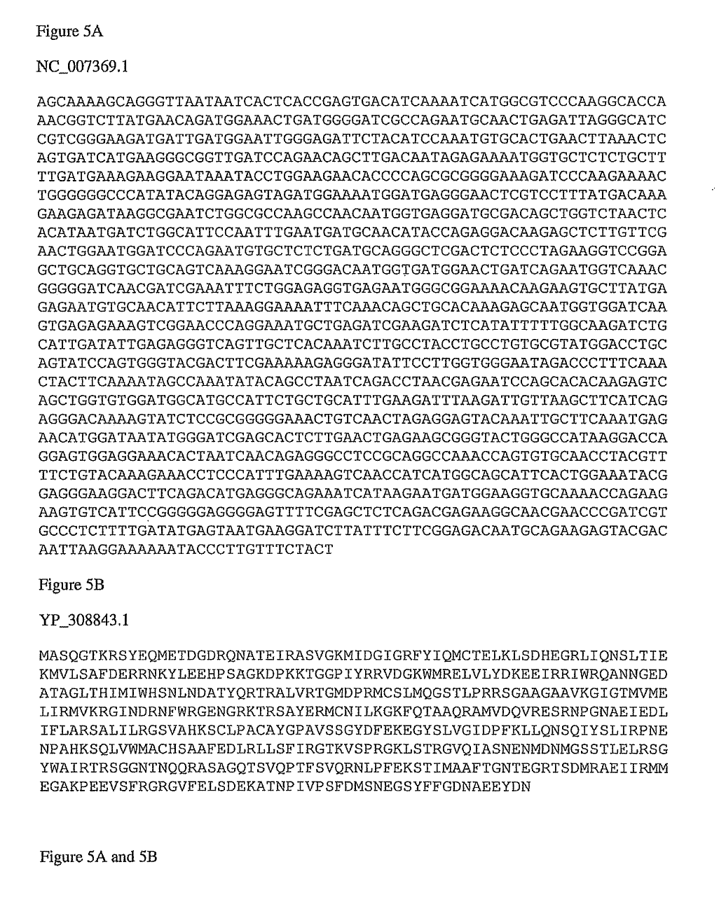

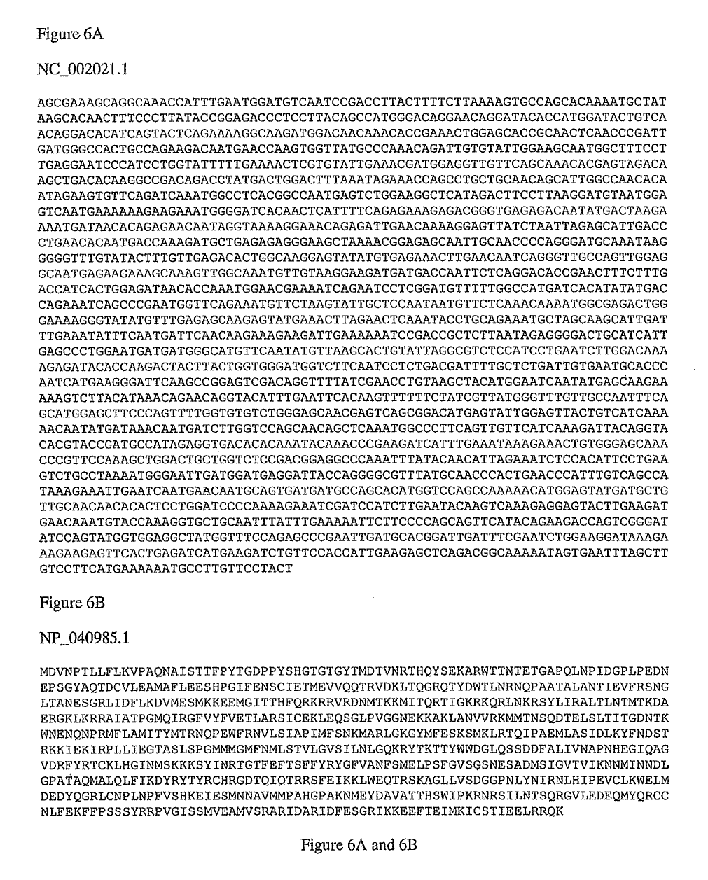

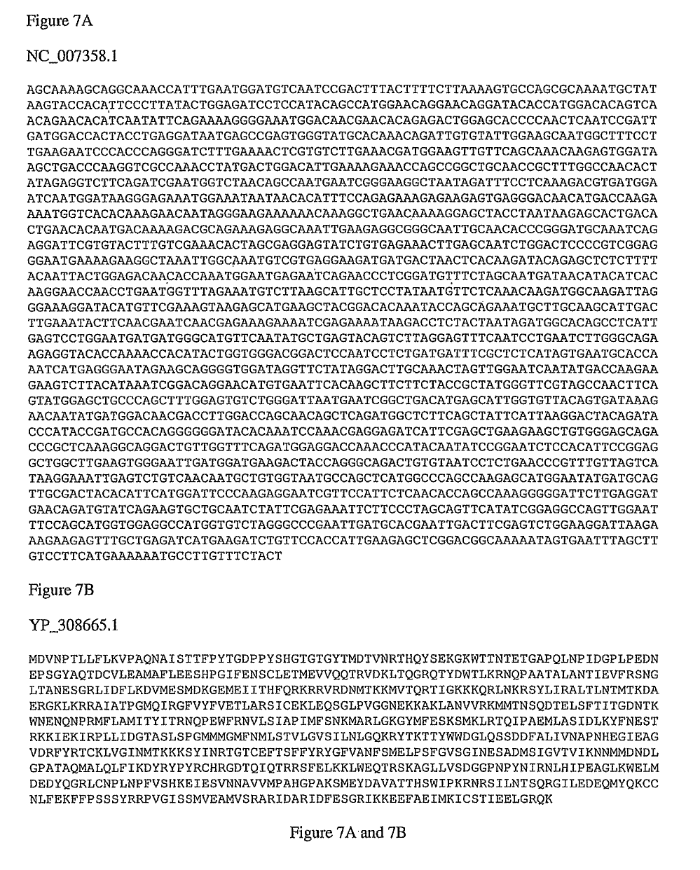

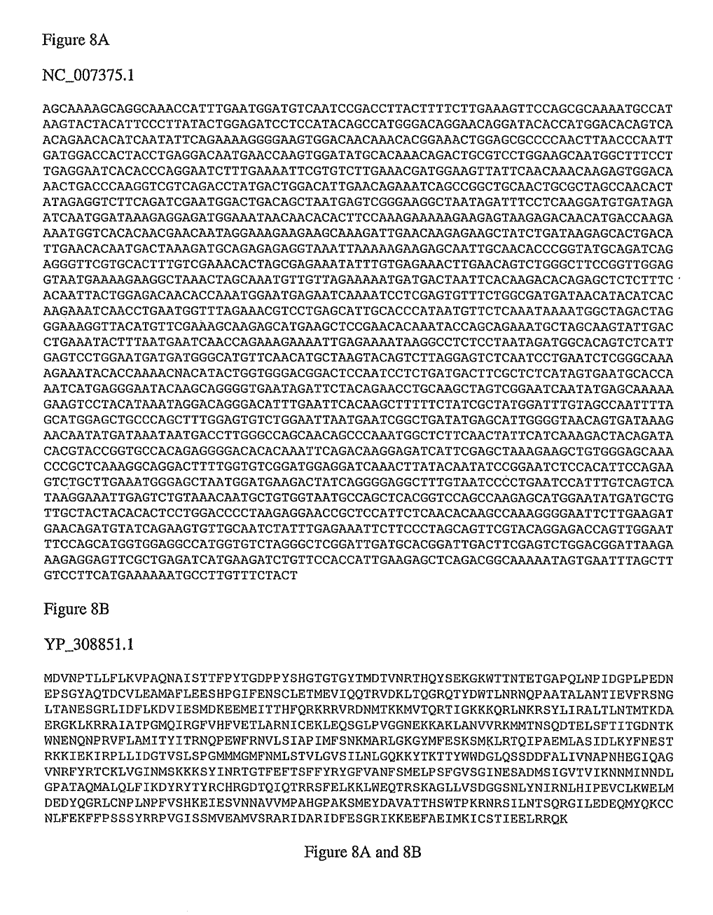

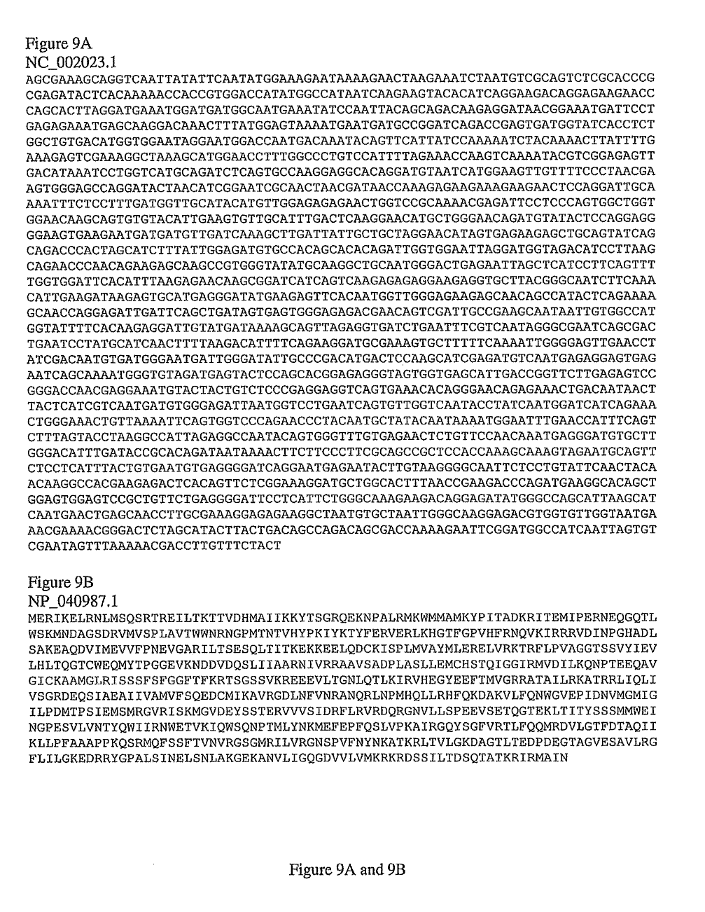

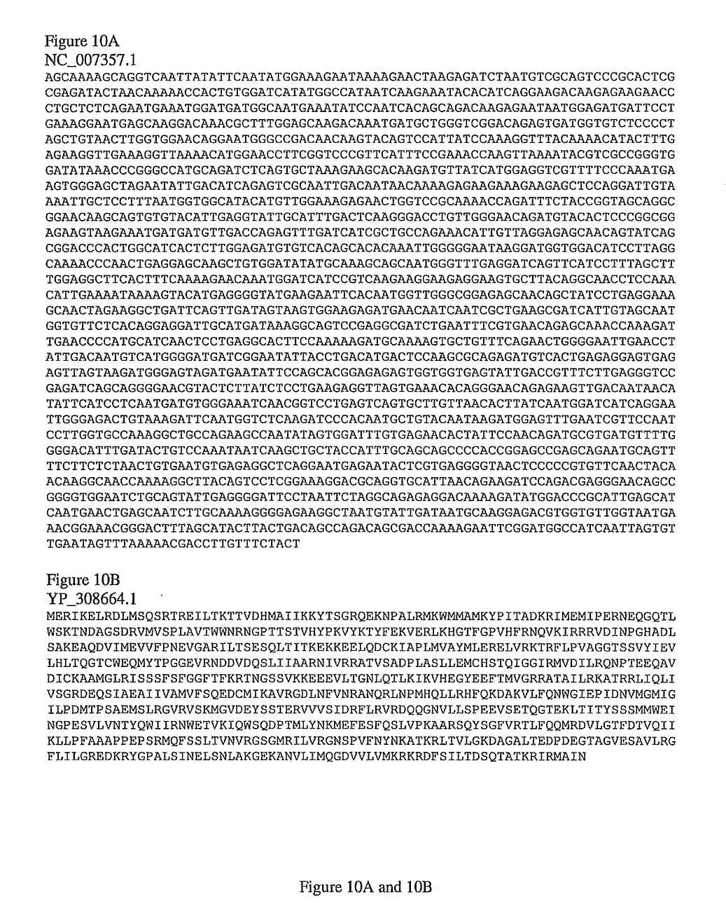

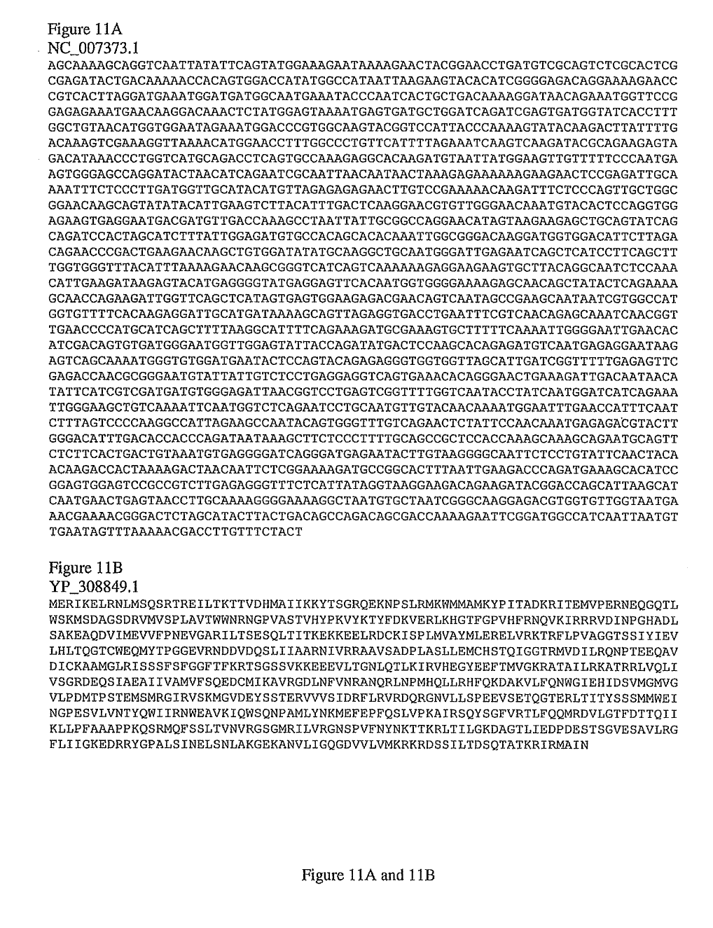

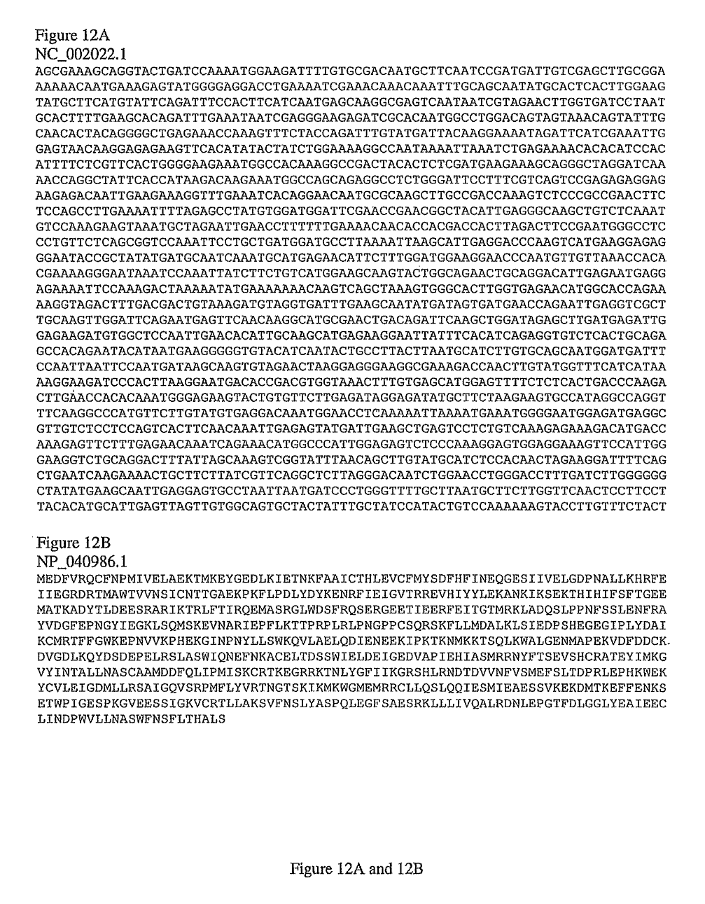

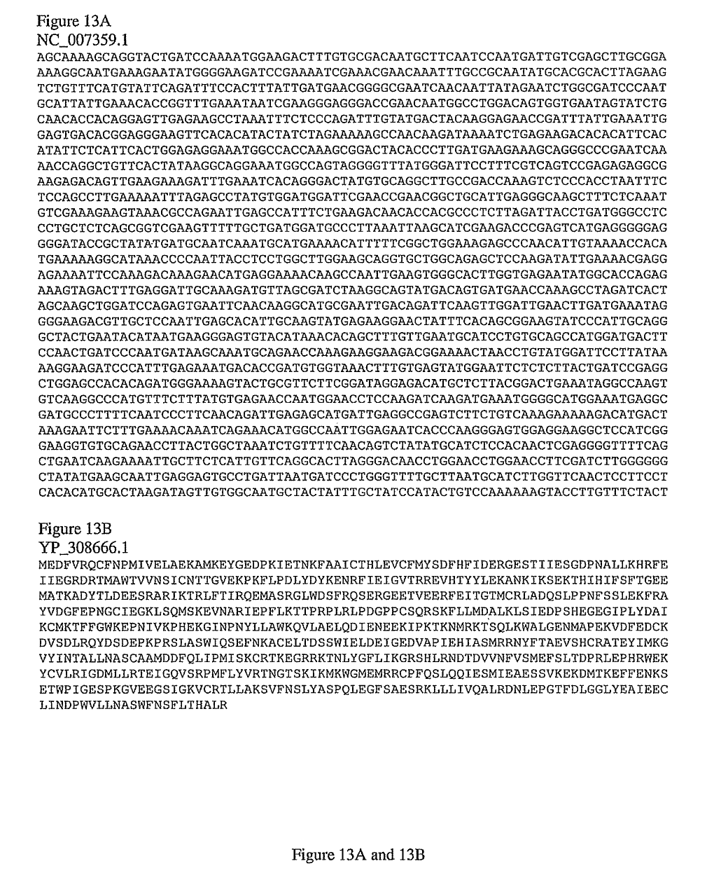

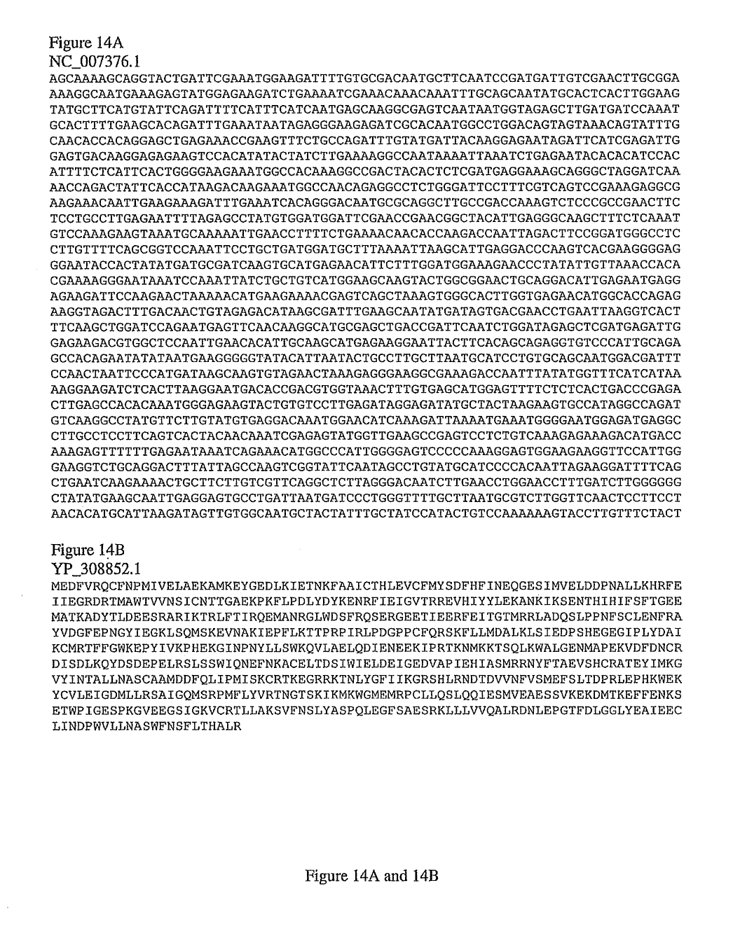

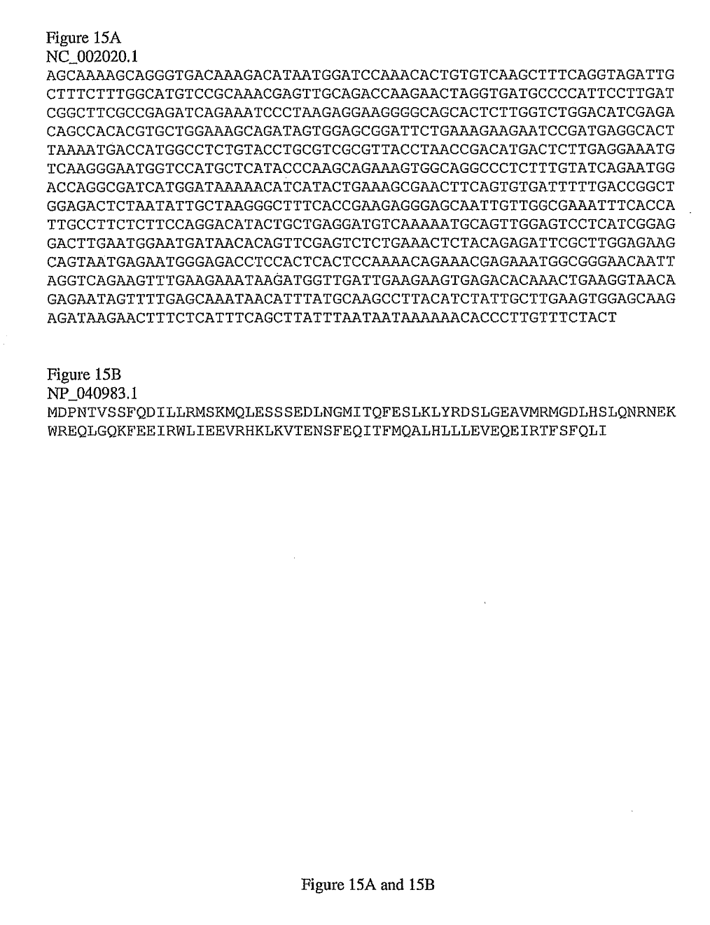

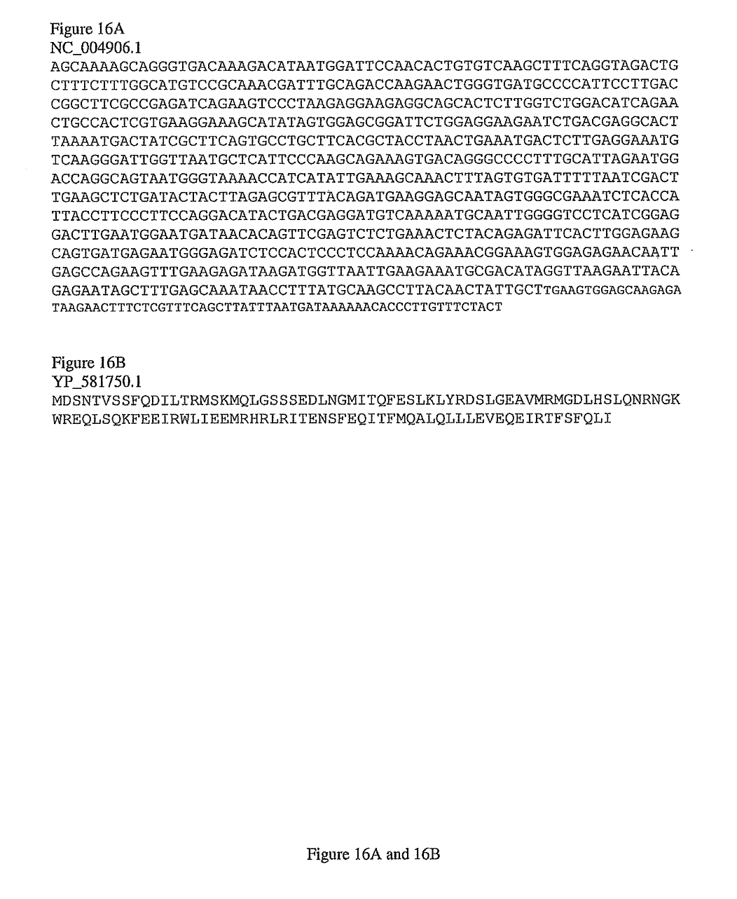

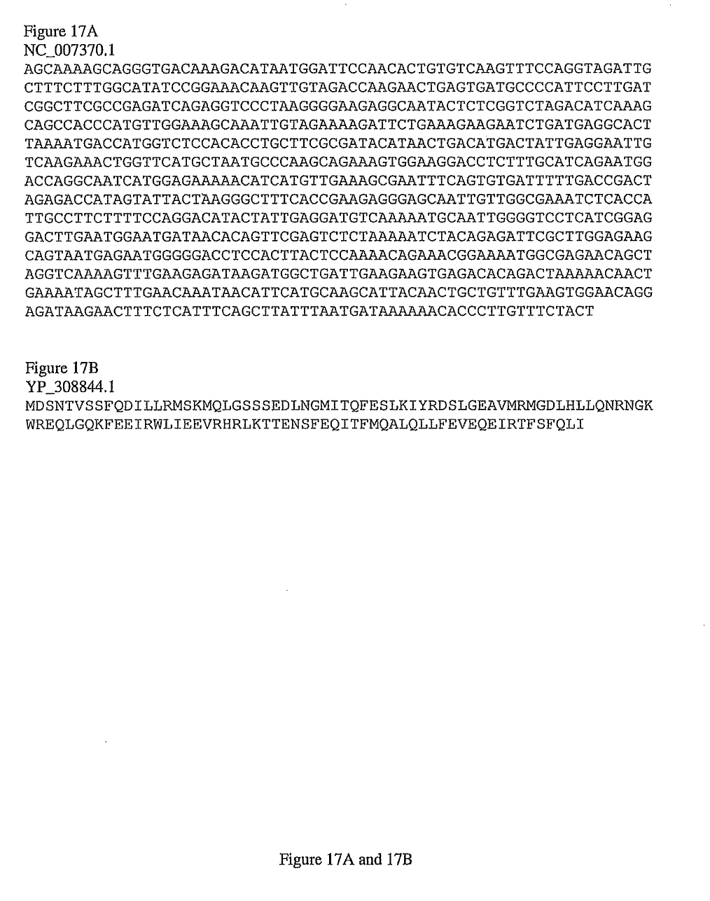

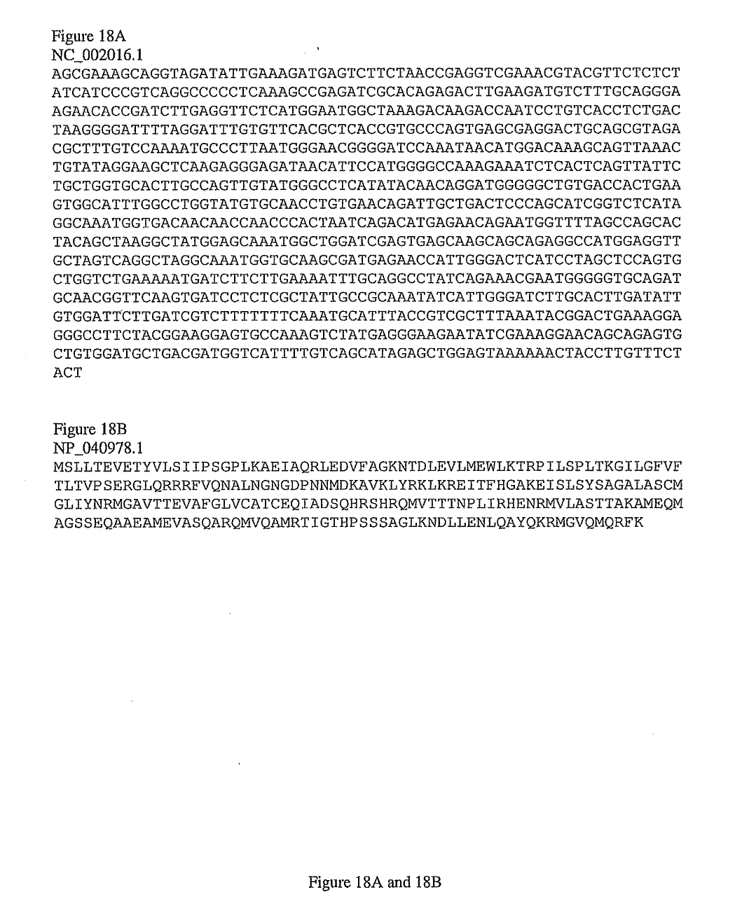

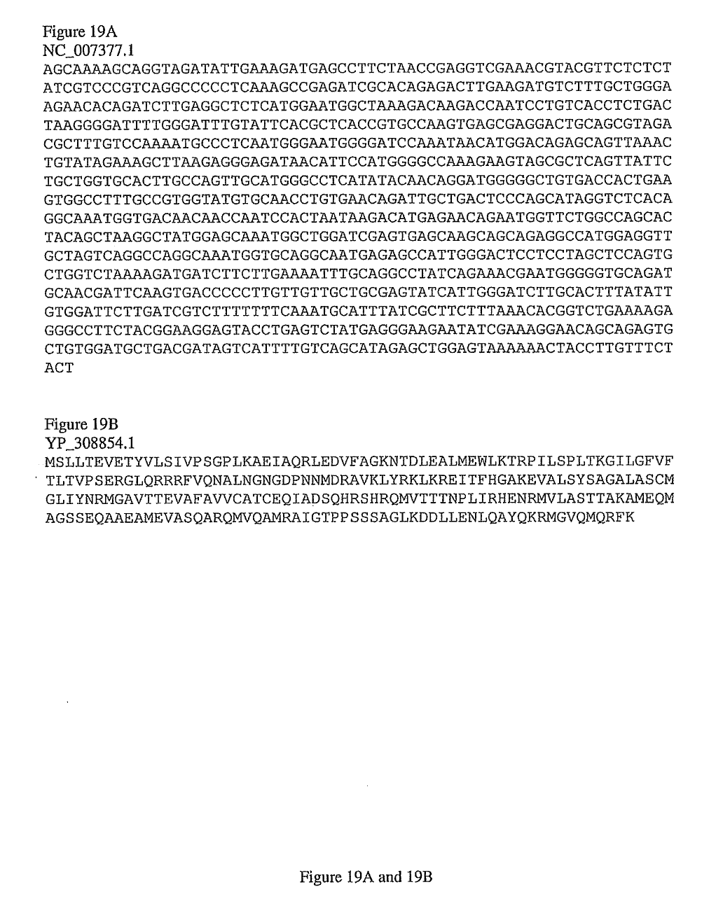

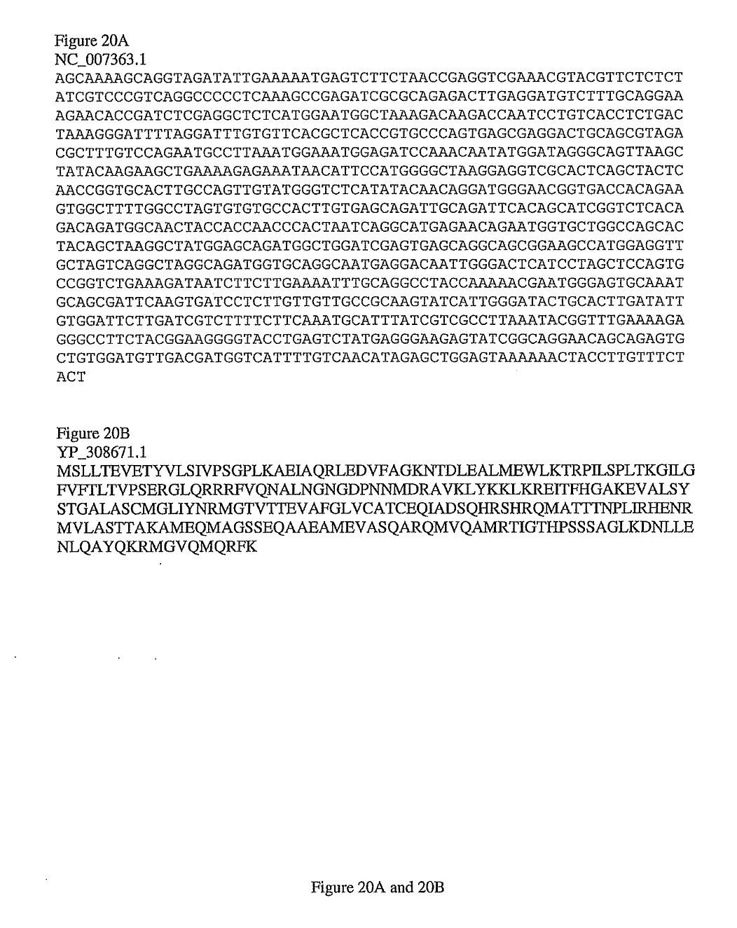

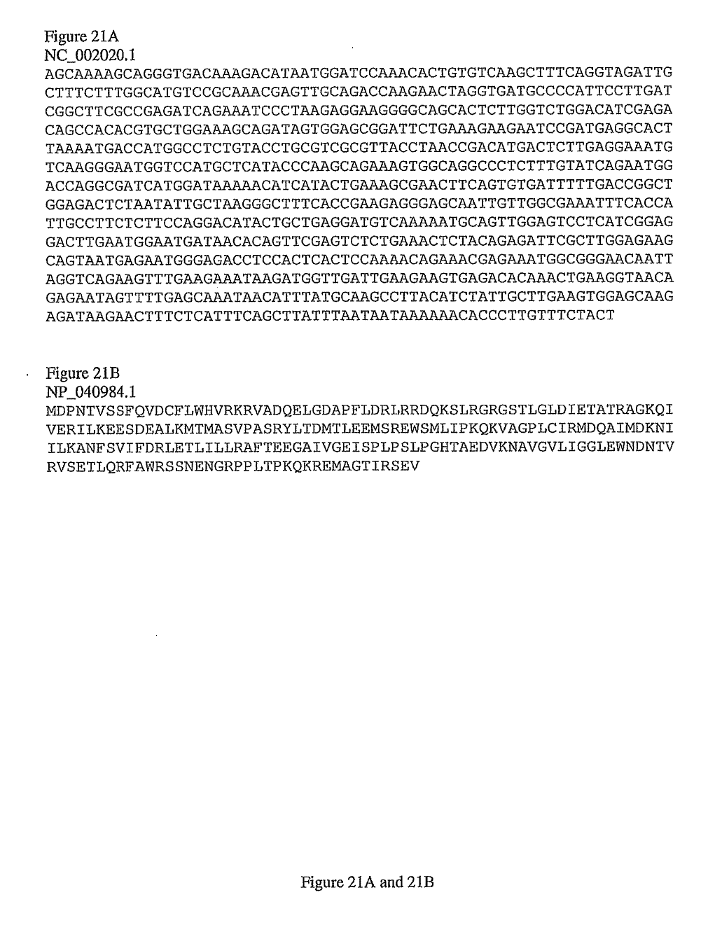

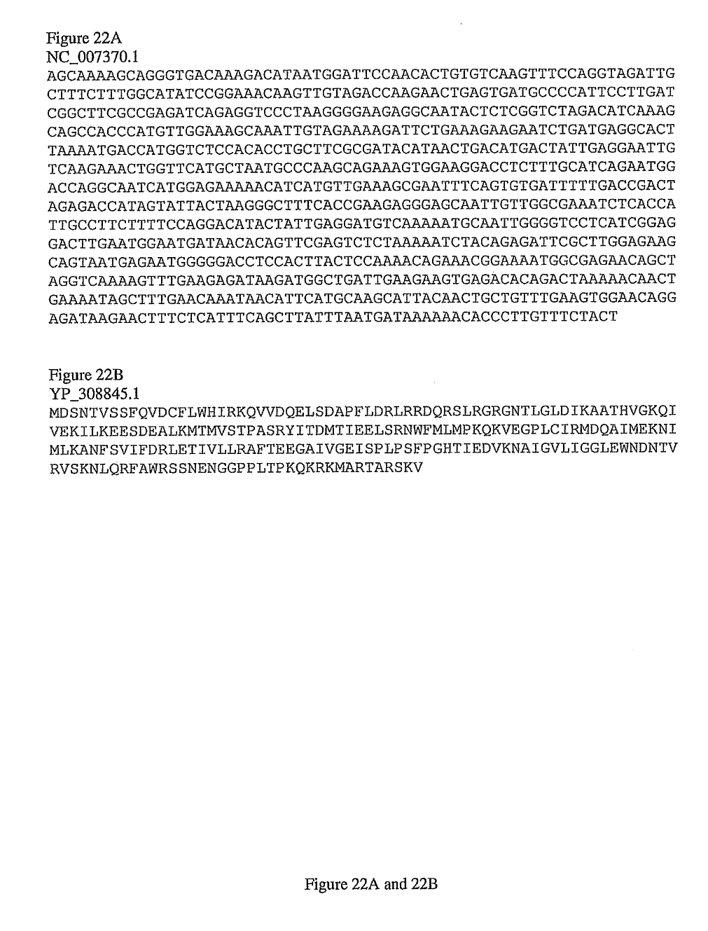

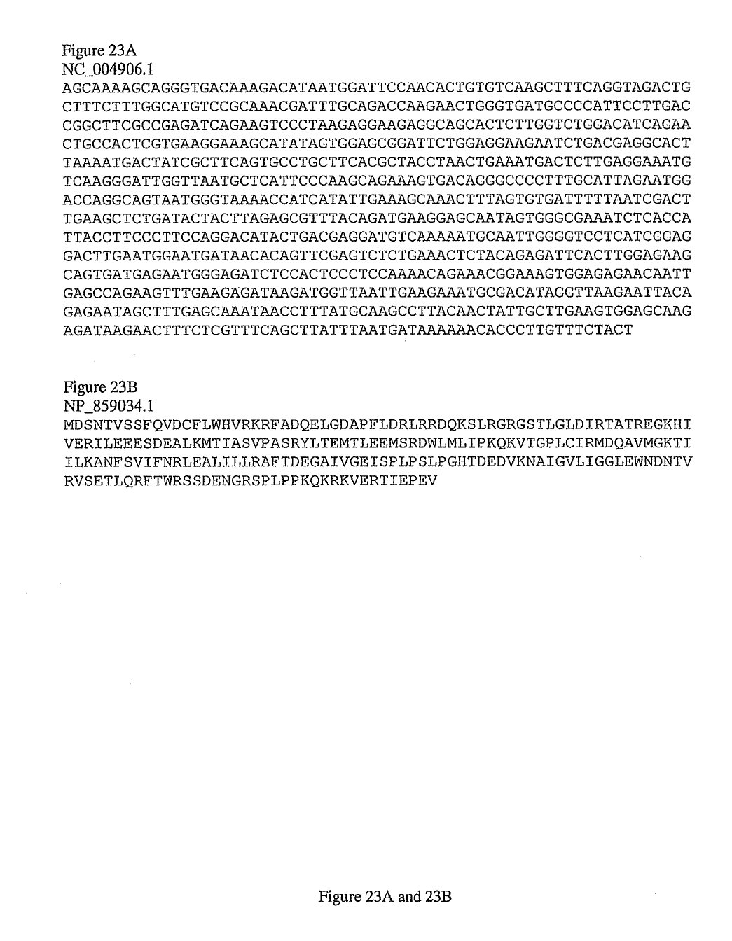

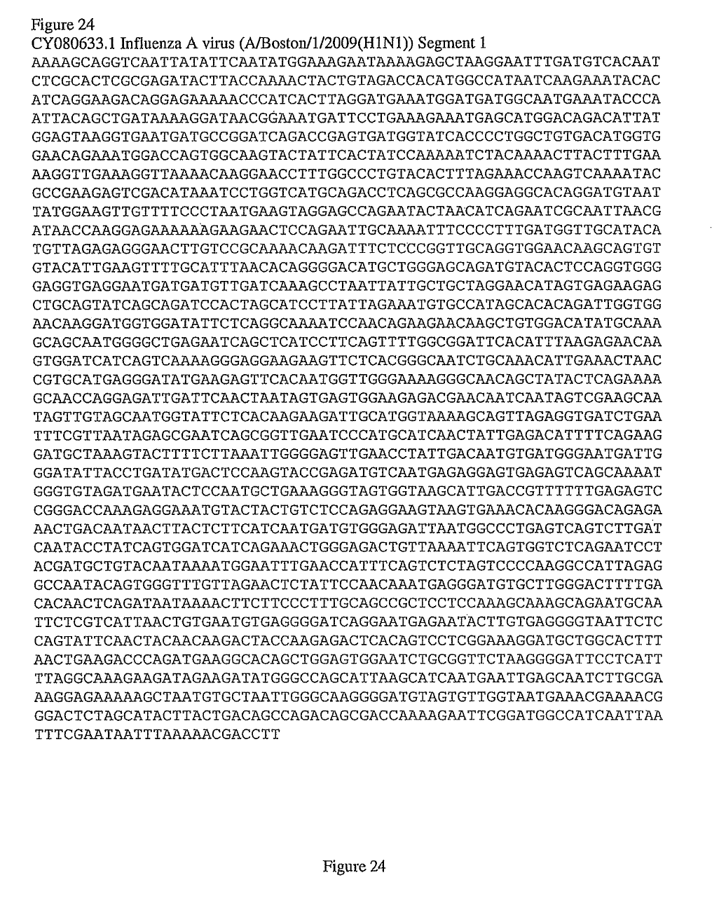

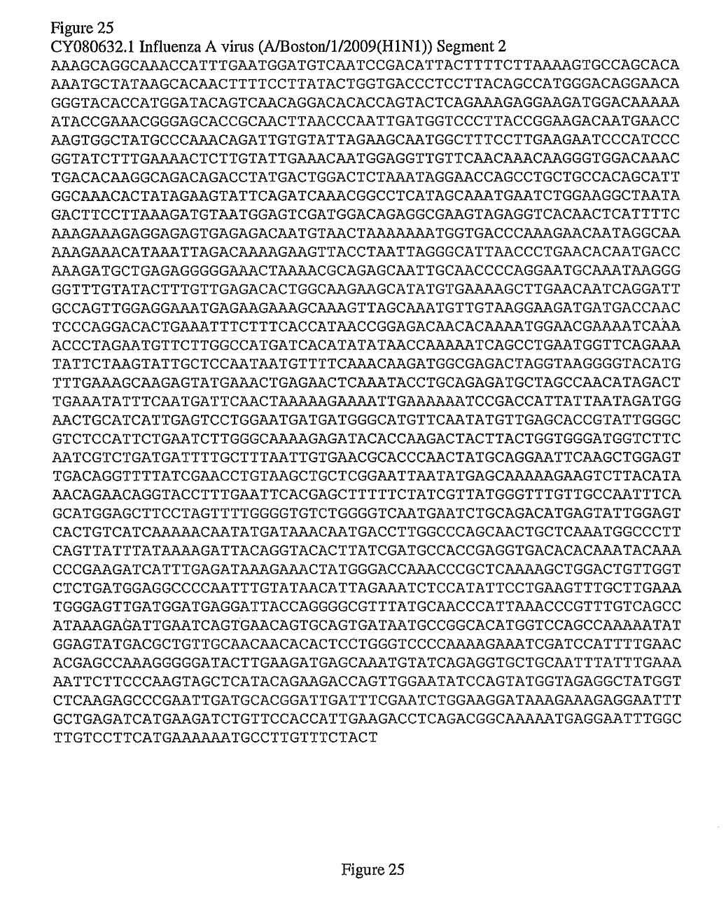

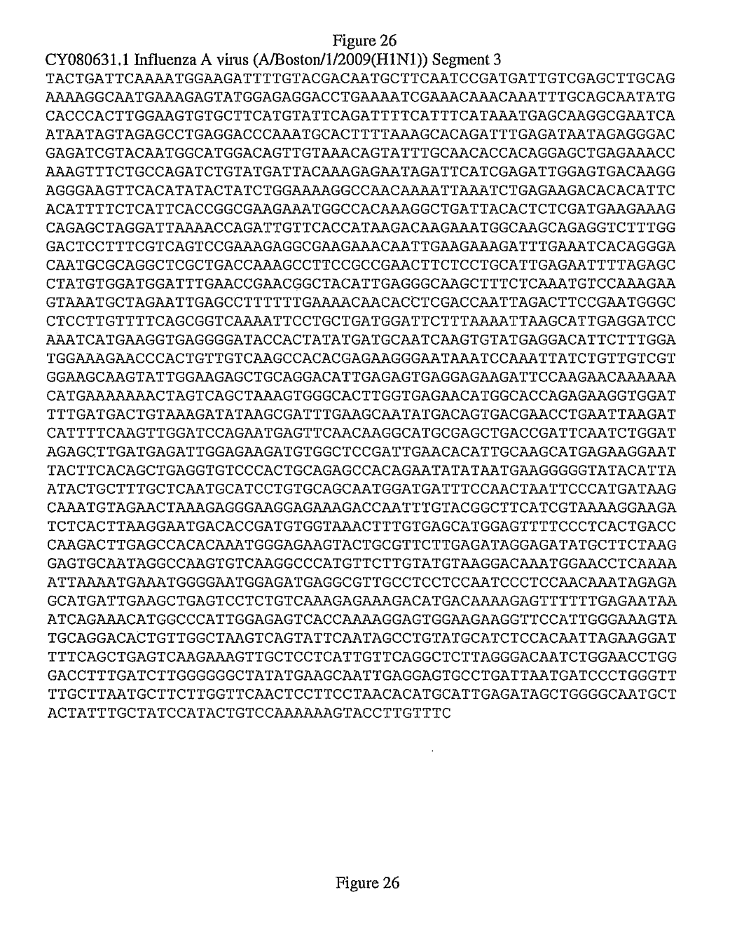

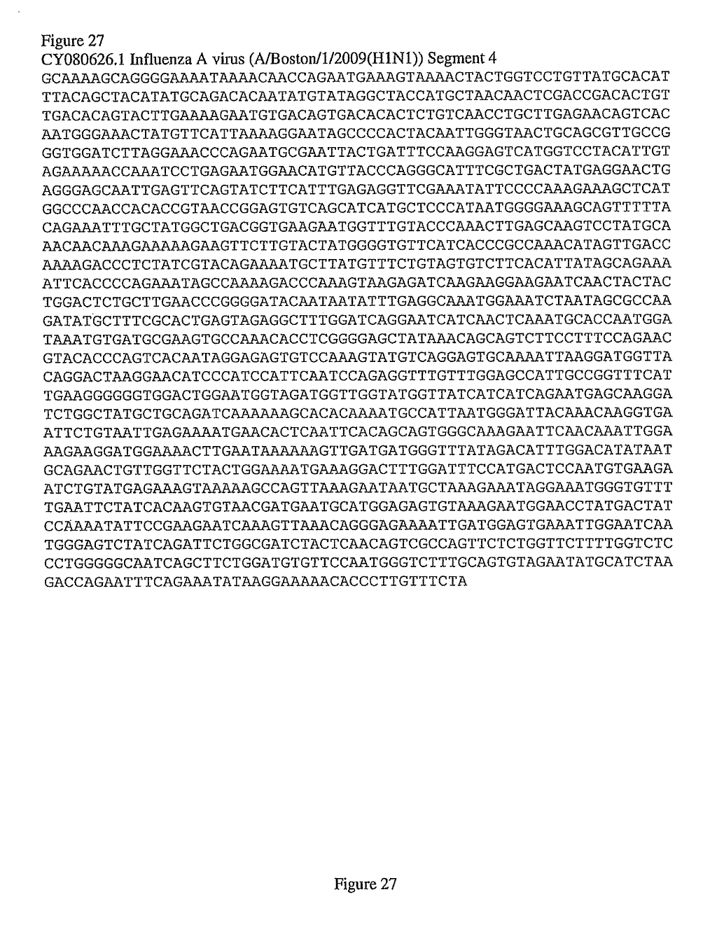

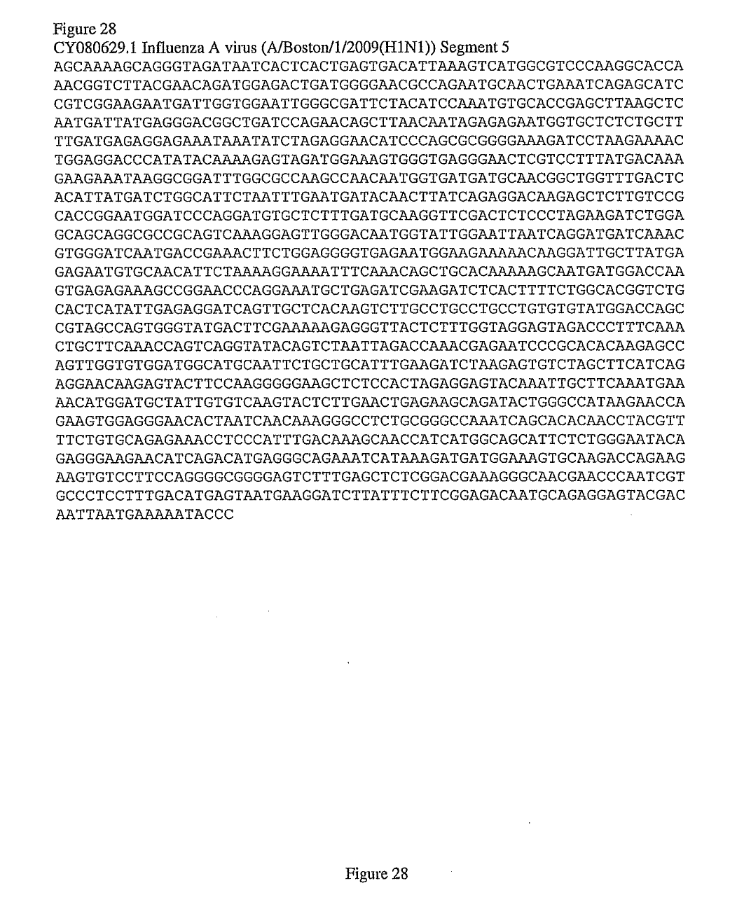

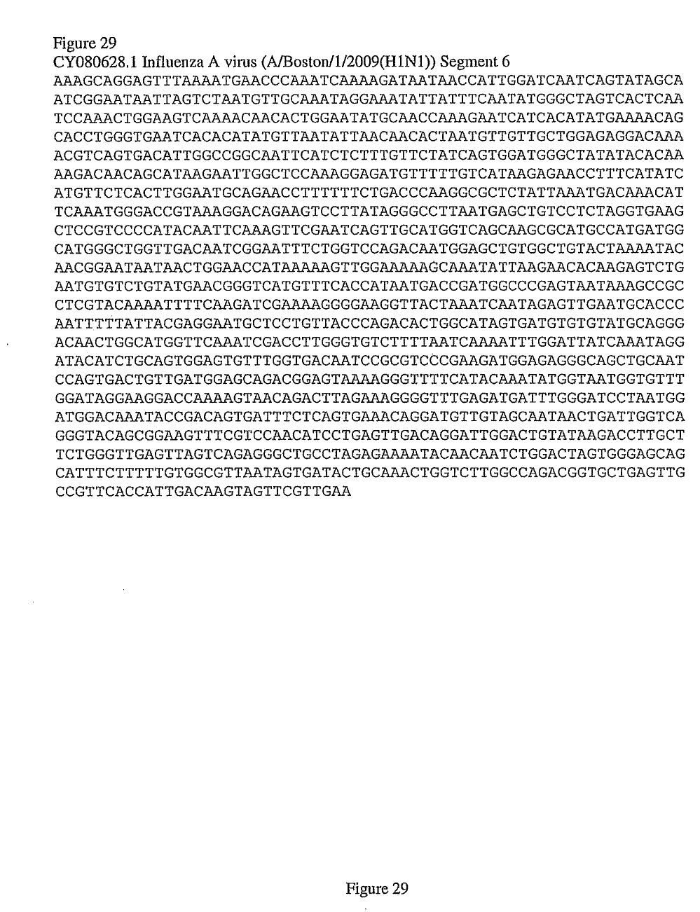

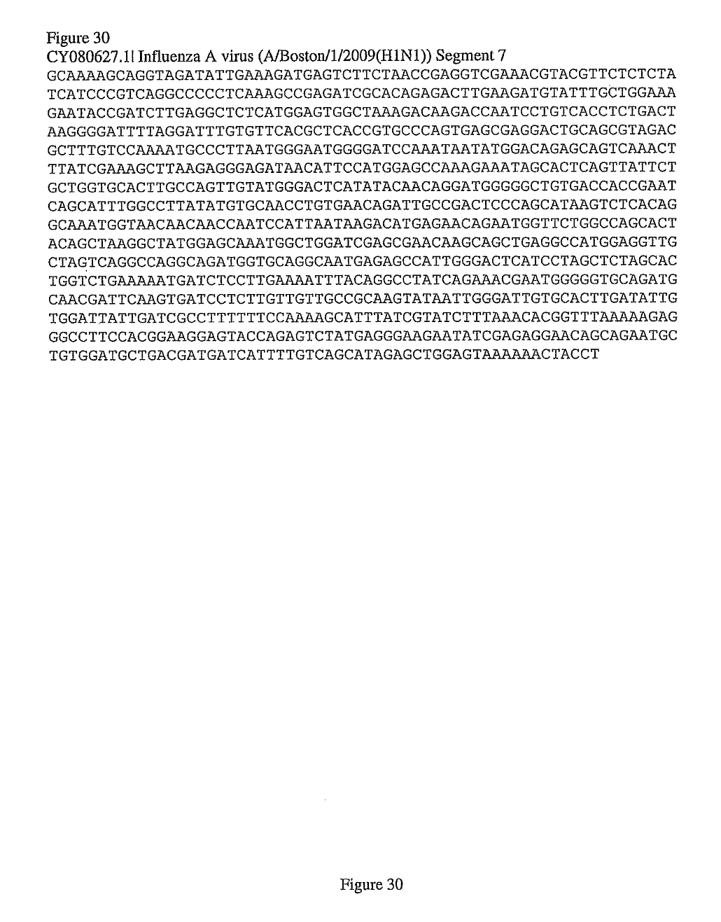

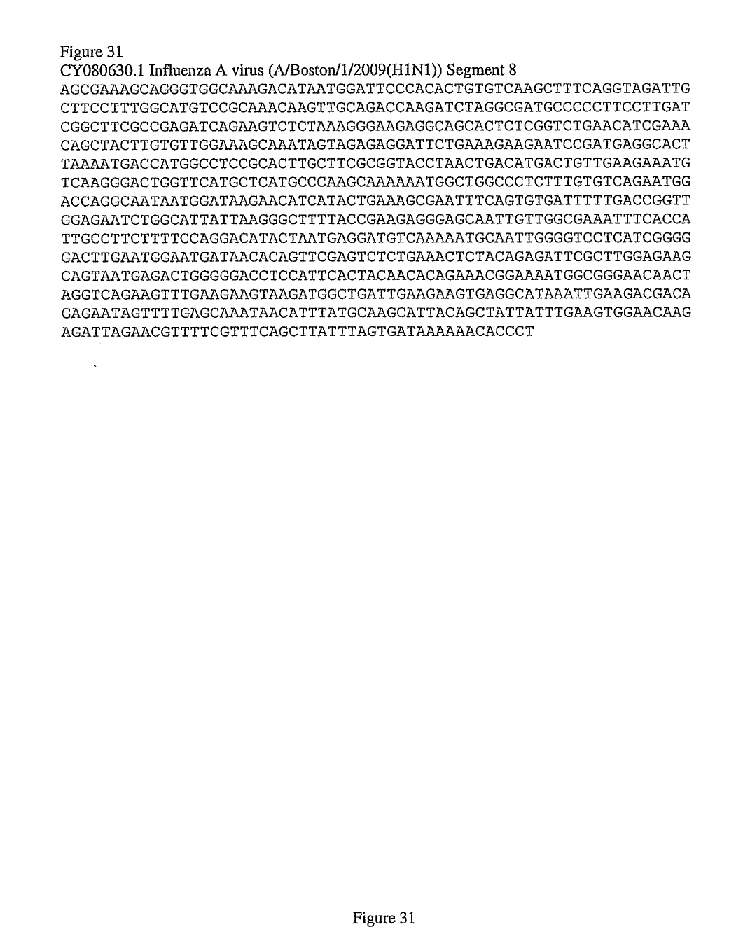

Characterization of influenza virus isolates are performed using an extensive collection of avian and swine influenza isolates. In particular, strain A/Sw/NC/38448-1/2005 is used as a template for recombinant nucleoprotein production as well as in homologous challenge experiments. Beginning in June 2005, H1N1 viruses of human-like HA genotype were isolated from swine. A/Swine/North Carolina/38448-1/2005 (ISDN126735), which is typical of the viruses isolated from North Carolina (NC) during that time period. The strain was isolated from 9 week old pigs with clinical signs of respiratory disease including coughing and nasal discharge. This virus and the others isolated from NC in 2005 are human-like H1N1 viruses that share >98% similarity to H1 human influenza viruses circulating in 2004 such as A/Poitiers/2168/2003 and <75% similarity with swine influenza reference strains. The 2005 swine influenza viruses with human-like H1 HA sequences share >97% similarity to each other but <95% similarity with the 2003 human/swine H1N2 reassortant viruses. Phylogenetic analyses suggest that the 2005 strains are the result of separate reassortment events between human and swine influenza viruses and is not a direct descendent of the 2003 strains (FIG. 1). Furthermore, the NA genotype of the 2005 human-like H1N1 is an N1 of human origin. Genetic analyses of the internal genes of both the 2003 H1N2 and the 2005 H1N1 human/swine reassortant viruses reveals M, NP, NS, PA, PB1 and PB2 genes similar to those of the triple reassortant swine H3N2 viruses. Triple reassortant H3N2 viruses are the dominant H3N2 strain circulating in pigs in the U.S. (Webby et al, 2000).

### Example 3

The immune-stimulatory effect and protective ability of VLP-NP given either transdermally or transmuscularly is evaluated in pigs. Two-week-old piglets are divided into groups of 10 pigs per challenge group and 5 pigs per control group (Table 1, FIG. 1). Pigs receive the vaccine at days 0 and 14 of the study. They are then challenged intranasally on day 28 of the study with homologous influenza strain: A/Swine/North Carolina/38448-1/2005 human-like H1N1. Blood samples are drawn prior to injection on days 3, 7, 14, 28, 35, and 42. Serum levels of anti antibodies (via HA inhibition against vaccine and challenge strains) are measured. Any concomitant treatment, morbidity, or mortality will be recorded. To assess potential immune stimulation afforded by IVP-NP, blood collected from pigs are evaluated for the following: (1) the numbers of circulating immune cells (macrophage, B cell, CD4, CD8 and T cell populations); (2) concentration of activated lymphocytes using CD45 and CD44 markers and (3) natural killer immune cell activity. All immune measures are assessed on peripheral blood mononuclear cells using flow cytometry. Half of the pigs (n=5) are euthanized at 5 days post challenge (dpc) and the remaining pigs are euthanized at 14 dpc. Clinical disease are measured and assessed on a daily basis using body temperature, respiratory signs, and weight gain as criteria. Lesions are assessed post-mortem. Virological measures are assessed both qualitatively and quantitatively using virus isolation and titration from swabs and tissue homogenates, with virus detection and subtype confirmed by RT-PCR. The optimum route is defined as the route that assures physiologic stimulation of the immune system to levels no more than 30% above control levels and a physiological increase in activated lymphocytes and/or natural killer cells.

TABLE 1
Experimental Design

| | | Vaccination | | Challenge |
|---|---|---|---|---|
| Group # | Vaccine dose | Route | # Animals | Strain |
| 1 | Maximum, | Transdermal | 10 | Homologous: |
| | 2X 2 weeks | | | human-like |
| 2 | Maximum, | Intramuscular | 10 | Homologous: |
| | 2X 2 weeks | | | human-like |
| 3 | None | N/A | 10 | Homologous: |
| | | | | human-like |
| 4 | None | N/A | 5 | None |

### Example 4

Pigs are divided into 5 groups (Table 2, FIG. 1). Pigs of 14 days of age remain in facilities from study day −3 to 21. Pigs are weighed on study days 3, 7, 14, 21, 28 and 35 to evaluate both weight gain and weight differences by group. They are bled on days 3, 14, 28, 35, and 42. IVP-NP treated pigs receive the vaccine via the optimum route determined on days 0 and 14 of the study. Pigs are intranasally challenged on day 28 of the study with one of two virulent SIV strains: heterosubtypic A/Swine/Colorado/00294/2001 H3N2 or heterologous A/Swine/Minnesota/00040/2002 reassortant H1N1 (rH1N1). Pigs in all groups are evaluated daily post-challenge or post-exposure until humane euthanasia. Blood samples are drawn prior to injection on day 3, 7, 14, 28, 35, and 42. Serum levels of anti-influenza antibodies are determined via HA inhibition against vaccine and challenge strains. Any concomitant treatment, morbidity, or mortality is recorded. To assess potential immune stimulation afforded by IVP-NP, blood collected from pigs is evaluated for the following: (1) the numbers of circulating immune cells (macrophage, B cell, CD4, CD8 and yo-T cell populations); (2) concentration of activated lymphocytes using CD45 and CD44 markers; and (3) natural killer immune cell activity. All immune measures are assessed on peripheral blood mononuclear cells using flow cytometry. At day 5 post-challenge, 5 pigs in each group (n=5) are euthanized to evaluate viral load in lungs and lesions. The remaining pigs are euthanized at 14 days post-challenge to evaluate the immune response to SIV post-challenge. Lesions are assessed post-mortem. Virological measures are assessed both qualitatively and quantitatively using virus isolation and titration from swabs and tissues homogenates, with virus detection and subtype confirmed by RT-PCR.

TABLE 2
Experimental Design

| | | | Challenge |
|---|---|---|---|
| Group | Vaccine Dose | N per group | strain |
| 1 | Maximum, 2X, 2 weeks apart | 10 | Heterologous |
| | hu-like H1N1 IVP-NP vaccine | | rH1N1 |
| 2 | Maximum, twice, 2 weeks apart | 10 | Heterosubtypic |
| | hu-like H1N1 VLP-NP vaccine | | H3N2 |
| 3 | None | 10 | rH1N1 |
| 4 | None | 10 | H3N2 |
| 5 | None | 5 | None |

### Example 5

The entire open reading frame of NP of SIVA/swine/Minnesota/0702083/2007 (H1N1) was amplified using RT-PCR and cloned into the pIEX/Bac EK/LIC vector (Novagen®, San Diego, Calif.) per the manufacturer's instructions. The NP recombinant baculovirus was produced in Sf9 insect cells using BacMagic-2 DNA kit (Novagen®, San Diego, Calif.) per the manufacturer's instructions. The NP recombinant baculovirus was amplified by adding the recombinant virus seed stock at a MOI of 0.025 to 200 ml culture of Sf9 cells at 2×10⁶ cells/ml in log phase and incubated at 26.5° C. for 4-5 days. The NP recombinant baculovirus titer was then measured by end-point dilution. The stock of virus was aliquoted and stored at −80° C.

The NP protein was expressed in bulk by infecting 10 liters of 2×10⁶ cells/ml of Sf9 cells with the NP recombinant baculovirus at a MOI of 5. The infected cells were incubated with shaking at 26.5° C. for 3 days. The supernatant, which contained the NP protein, was collected, purified, and concentrated by ion exchange. The NP protein was then aliquoted and stored at −80° C. The expressed NP protein was verified by several techniques including SDS-PAGE, Western Blot and mass spectrometry.

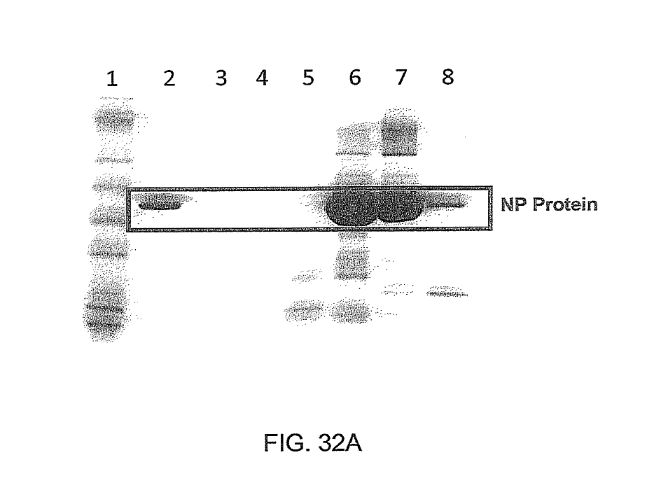

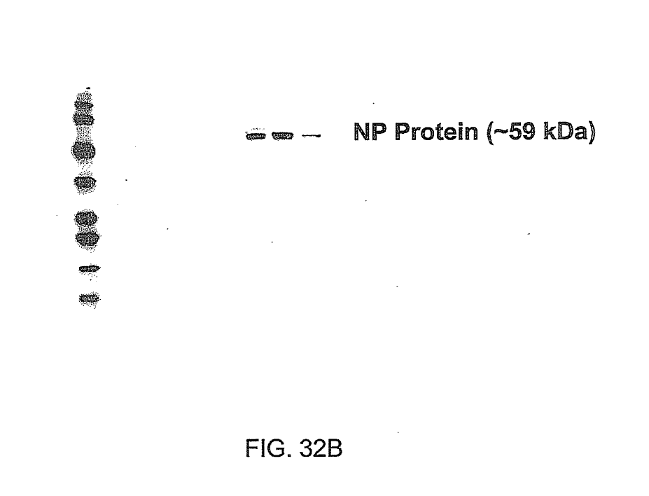

FIG. 32A illustrates a Coomassie stained SDS-PAGE gel of a NP anion exchange gradient elution. Lane 1 was loaded with a protein marker. Lanes 2 and 3 were loaded with the load and flow through/wash, respectively. Lane 4 was blank. Lanes 5-8 were loaded with eluate fractions 4-7, respectively, and expressed NP was observed primarily in fractions 5-7. FIG. 32B is a Western Blot of the SDS-PAGE gel shown in FIG. 32A confirming that NP is the expressed protein.

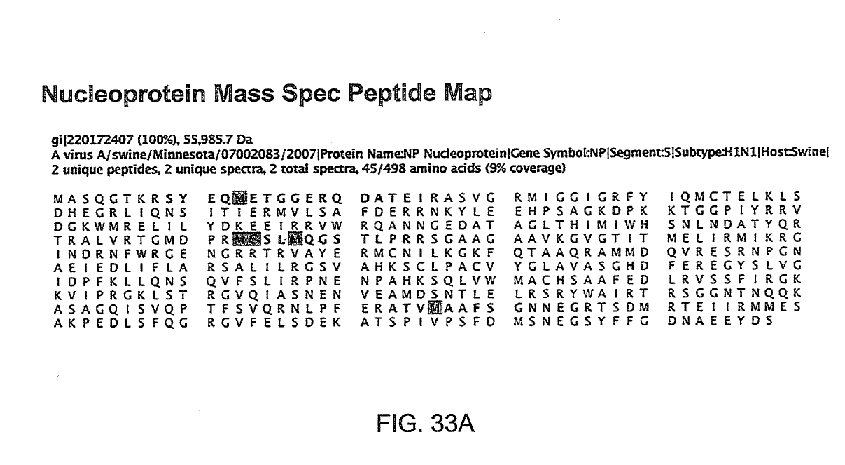

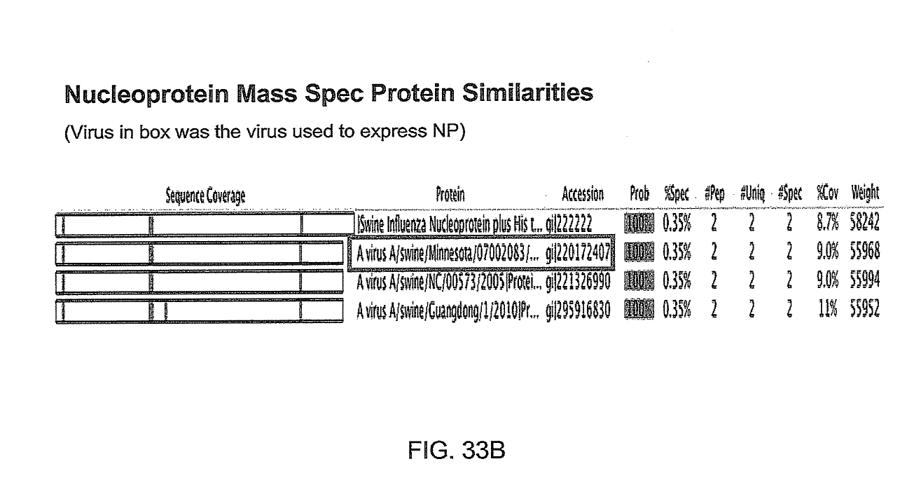

The identity of the expressed protein was further confirmed by mass spectrometry. As shown in FIG. 33A, 3 unique peptides were identified, which together account for 45/498 amino acids of the NP protein. A similarity analysis was conducted and a 100% correlation with the strain used to express the NP protein was observed (see, e.g., the box in FIG. 33B).

### Example 6

The role of NP protein in inducing a serological response was evaluated in sera taken from animals in three vaccination-challenge trials. A total of 384 sera were screened from the three trials. An anti-NP antibody response was delayed in vaccinated animals, but appeared rapidly after challenge, indicating that NP is antigenically presented in the host animal. The rapid identification of anti-NP antibodies post-challenge in this experiment indicates that NP ELISA may be used to discriminate between infected and vaccinated animals.

### Example 7

An indirect nucleoprotein (NP) ELISA was optimized using swine influenza virus (SIV) positive swine sera (Table 1) and NP-free swine sera (Table 2). The purified NP protein was coated onto separate 96-well plates in 0.05 M carbonate bicarbonate buffer, pH 9.6, overnight at 4° C. The NP concentrations tested were 50 ng/well (0.5 μg/ml), 100 ng/well (1 μg/ml) and 500 ng/well (5 μg/ml). The serum dilution tested were 1:10, 1:100 and 1:1000. The wells were washed three times with 0.05% Tween-20 phosphate buffered saline (PBST) and subsequently blocked with 5% nonfat milk powder in PBST for 30 minutes at 37° C. The positive and negative sera were added, followed by incubation for 1 hour at room temperature (RT). The plates were then washed three times with PBST, followed by the addition of goat anti-pig IgG HRP secondary antibody diluted 1:10,000 and 1:50,000 in PBST. After 1 hour incubation at RT, the plates were washed 3 times with PBST and 100 μL/well of TMB substrate was added. After 15 minutes incubation at RT, the reaction was stopped by adding 50 μL/well of 2M sulfuric acid. The optical density at 450 nm was measured with an automated plate spectrophotometer.

### Example 8

Recombinant NP was inoculated subcutaneously in 10 specific pathogen free pigs for follow-up and analysis of immune responses and potential protection from challenge.

Serum from pig 574 and H3N2 serum showed very low to no signal reactivity to NP. At 1:10,000 and 1:50,000 goat anti-pig IgG HRP, signal for pig 880 and pig 886 decreased at 1:10 serum dilution while signal increased at 1:1000 serum dilution as NP concentration increased. At 1:100 serum dilution and 1:10,000 goat anti-pig IgG HRP, the optical density for pig 880 ranged from 0.9 to 1.1 while that of pig 886 ranged from 1.4 to 1.6. At 1:100 serum dilution and 1:50,000 goat anti-pig IgG HRP, the optical density for pig 880 ranged from 0.6 to 0.8 while that of pig 886 ranged from 0.8 to 1.1.

TABLE 1
List of SIV swine sera tested for use as positive control

| Pig ID | Strain |
|---|---|
| 880 | A/Swine/North |
| | Carolina/02084/2008 β H1N1 |
| 886 | A/Swine/Minnesota/02011/2008 |
| | δ1 H1N2 |
| 574 | A/California/04/2009 pH1N1 |
| H3N2 | Pfizer H3N2 crude serum |
| Serum | |

TABLE 2
List of SIV swine sera used as negative control

| Pig ID | Strain |
|---|---|
| 54 | Serum collected after vaccination with H3 |
| | protein from A/Swine/Minnesota/Sg-00235/2005 H3N2 |
| 52 | SIV free serum |

### Example 9

To assess the ability to use an internal moiety of an influenza virus as a vaccine, pigs were vaccinated either intradermally or intramuscularly with the NP/Matrix ectodomain.

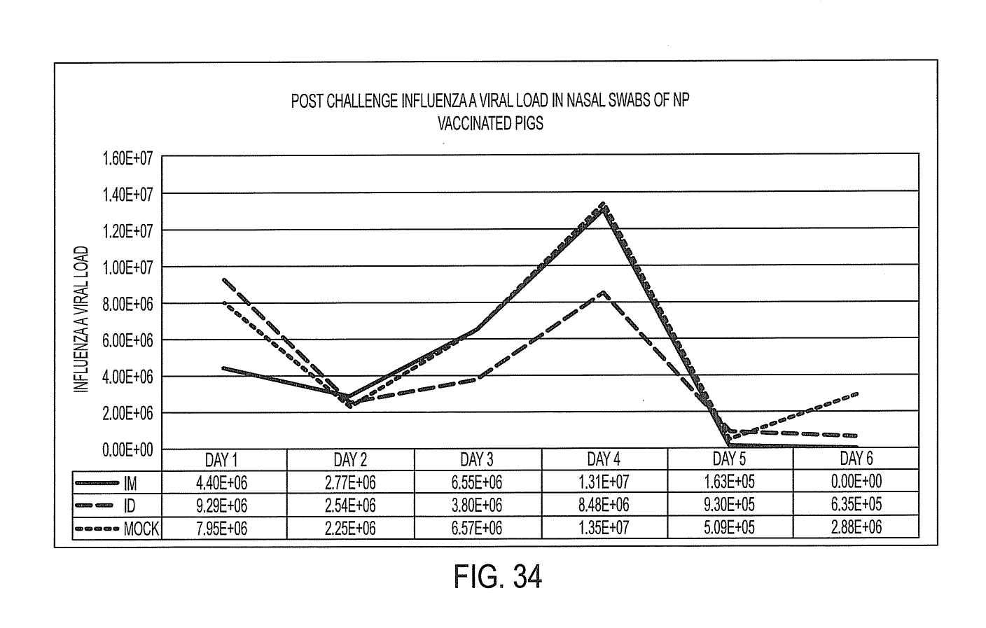

Nasal swabs were collected post-vaccination to evaluate reduction in viral loads. The viral loads were measured using realtime RT-PCR on nasal swabs collected from vaccinated pigs days 1-6 post challenge. As shown in FIG. 34, the intradermally vaccinated pigs showed a reduced nasal shedding trend, while those vaccinated intramuscularly did not differ from controls (mock vaccinated group).

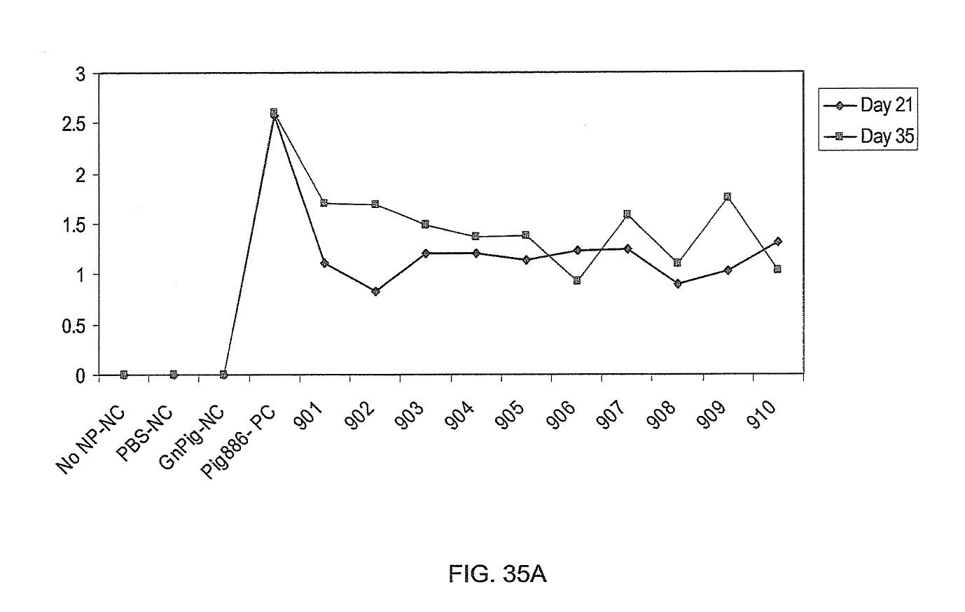

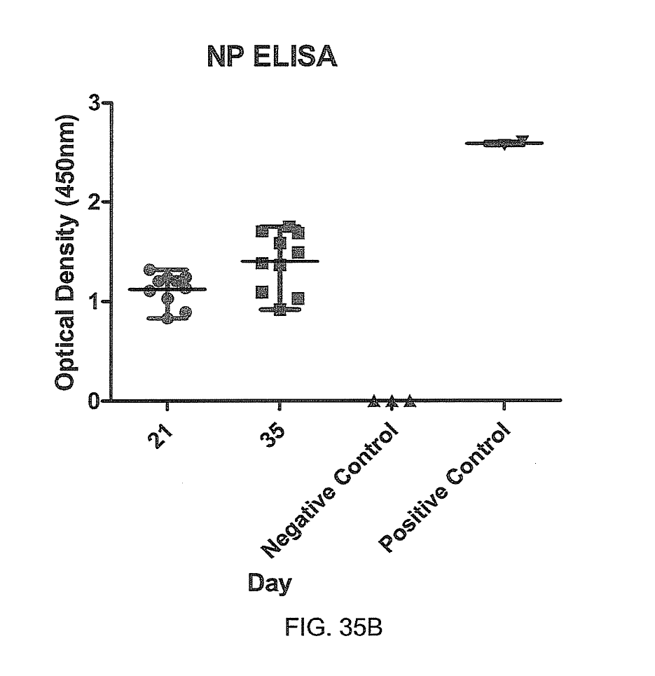

Blood was collected at 21 and 35 days post-vaccination and used to assess antibody titers. As shown in FIG. 35A, studies with the above described indirect ELISA assay to detect NP specific antibodies showed consistent increase in NP antibodies 21 and 35 days post vaccination, suggesting an immune recognition and response to intradermally administered NP. Similarly, the increase in NP antibodies 21 and 35 days post vaccination was consistent among all 10 vaccinated pigs, as shown in FIG. 35B, which illustrates the distribution of individual pig antibody titers.

### Example 10

To assess T-cell proliferation, Buffy coats were isolated from all test animals and labeled with 5-(and 6)-Carboxyfluorescein diacetate succinimidyl ester (CFSE) and incubated for 5 days with nucleoprotein (NP) (5.0 μg/mL), M2E peptide (5.0 μg/mL), H1N1 virus (256 HA units), and controls vehicle (10% DMSO; negative control) and concanamycin A (ConA) (5.0 μg/mL; positive control) at 37° C. in a humidified chamber containing 5% CO₂. Upon completion of incubation times, cells were also stained with CD3 in order to determine T cell populations.

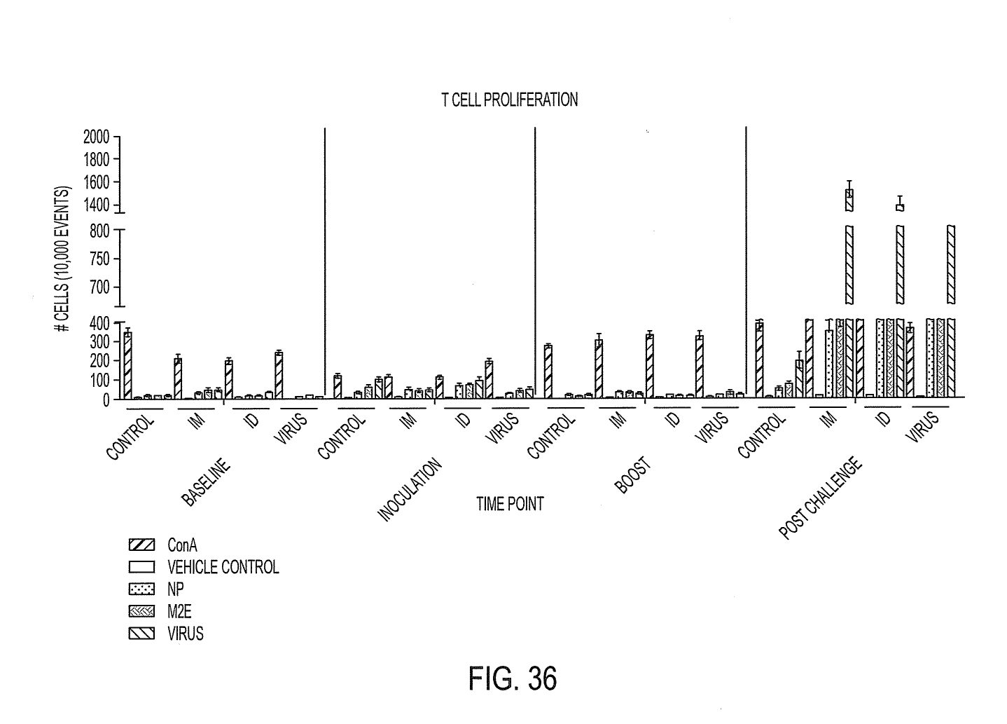

Ten thousand cell events were recorded on a BD FACS Canto and data were analyzed using Flowjo software. Cells were gated for divided T cell populations (CD3 high, FITC low) and treatment group numbers were recorded for all time points. As shown in FIG. 36, baseline T cell numbers showed no division in response to M2E, NP, M2E, H1N1, and vehicle control. In contrast, the positive control, ConA, showed 10% proliferation (percentage based on recorded lymphocyte population) indicating functional T cells capable of proliferation. Inoculation and booster time points revealed similar divided T cell numbers as baseline, suggesting that T cells did not respond to NP despite inoculation. However, post-challenge numbers show T cell proliferation in intramuscular (IM), intradermal (ID) and virus (H1N1 infected only) groups in comparison to the control group indicating a memory response due to prior exposure. It is also important to note that T cells from IM, ID, and virus groups post-challenge proliferate in response to NP and M2E stimulation. These results reflect prior exposure and memory to the entire virus. Importantly, intradermally delivered NP and M2E do effectively stimulate T cell division, which indicates that during the course of a viral infection T cells are exposed to and recognize NP and M2E.

1. An immunogenic composition comprising an effective amount of at least one internal moiety of an enveloped virus, or antigenic fragment thereof, and a pharmaceutically acceptable carrier or excipient.

2. The immunogenic composition of claim 1, wherein the internal moiety is any one or more viral polypeptides or polynucleotides selected from the group consisting of the RNA genome (RNAg), nucleoprotein (NP), RNA polymerase (PA), RNA polymerase 1 (PB1), RNA polymerase 2 (PB2), non-structural protein 1 (NS1), non-structural protein 2 (NS2), and matrix protein 1 (M1).

3. The immunogenic composition of claim 1, wherein the enveloped virus is selected from the group consisting of arenaviridae, arteriviridae, asfarviridae, baculoviridae, bornaviridae, bunyaviridae, coronaviridae, filoviridae, flaviviridae, hepadnaviridae, herpesviridae, orthomyxoviridae, paramyxoviridae, poxviridae, retroviridae, rhabdoviridae, and togaviridae.

4. The immunogenic composition of claim 3, wherein the enveloped virus is influenza.

5. The immunogenic composition of claim 1, wherein the at least one internal moiety comprises NP.

6. The immunogenic composition of claim 1, wherein the at least one internal moiety comprises NP and RNAg.

7-8. (canceled)

9. An immunogenic composition comprising an effective amount of a naked viral core particle of an enveloped virus, and a pharmaceutically acceptable carrier or excipient wherein the enveloped virus is influenza and the naked viral core particle is any one or more viral polypeptides or polynucleotides selected from the group consisting of the RNA genome (RNAg), nucleoprotein (NP), RNA polymerase (PA), RNA polymerase 1 (PB1), RNA polymerase 2 (PB2), non-structural protein 1 (NS1), and non-structural protein 2 (NS2).

10-12. (canceled)

13. The immunogenic composition of claim 9, wherein the naked viral core particle comprises at least three viral polypeptides or polynucleotides selected from the group consisting of RNAg, NP, PA, PB1, PB2, NS1, and NS2.

14-16. (canceled)

17. An immunogenic composition comprising an effective amount of at least one virus like particle (VLP) of an enveloped virus and a pharmaceutically acceptable carrier or excipient, wherein the enveloped virus is influenza and the VLP comprises any one or more viral polypeptides or polynucleotides, or antigenic fragments thereof, selected from the group consisting of RNA genome (RNAg), nucleoprotein (NP), RNA polymerase (PA), RNA polymerase 1 (PB 1), RNA polymerase 2 (PB2), non-structural protein 1 (NS1), and non-structural protein 2 (NS2).

18-20. (canceled)

21. The immunogenic composition according to claim 17, wherein the antigenic fragment is selected from the group consisting of a N-terminal, a C-terminal, and an internal polypeptide fragment.

22. An immunogenic composition comprising an effective amount of at least one recombinant fusion protein of an enveloped virus and a pharmaceutically acceptable carrier or excipient.

23. The immunogenic composition according to claim 22, wherein the at least one recombinant fusion protein comprises a protein, or antigenic fragment thereof, selected from the group consisting of nucleoprotein (NP), RNA polymerase (PA), RNA polymerase 1 (PB1), RNA polymerase 2 (PB2), non-structural protein 1 (NS1), non-structural protein 2 (NS2), and matrix protein 1 (M1).

24. The immunogenic composition according to claim 23, wherein the at least one recombinant fusion protein is joined with a viral polypeptide, or antigenic fragment thereof, selected from the group consisting of hemagglutinin (HA), neuraminidase (NA), and matrix protein 2 (M2).

25. The immunogenic composition according to claim 23, wherein the at least one recombinant fusion protein consists of NP, or an antigenic fragment thereof, joined with HA, or an antigenic fragment thereof.

26. A method of synthesizing naked viral core particles from enveloped viruses, said method comprising the production of live virus from a host or cell culture system, purification of the live virus from the host, and removal of the envelope from the purified live virus.

27-28. (canceled)

29. A method of inducing immunity against an enveloped virus in a mammal, said method comprising administering said mammal with an immunologically effective amount of an immunogenic composition according to claim 1.

30. The method of claim 29, wherein said method of administration is selected from the group consisting of topical, oral, anal, vaginal, mucosal, intravenous, intraperitoneal, intramuscular, subcutaneous, intranasal, nasopharyngeal, buccal, and intradermal administration.

31. A method of generating an immune response in a subject, the method comprising administering to said mammal an antigenic amount of an immunogenic composition of claim 1.

32. An influenza vaccine comprising:
a therapeutically or prophylactically effective amount of a naked viral core particle, wherein the naked viral core particle is comprised of any combination of one or more of the RNA genome (RNAg), nucleoprotein (NP), RNA polymerase (PA), RNA polymerase 1 (PB1), RNA polymerase 2 (PB2), non-structural protein 1 (NS1), and non-structural protein 2 (NS2); a pharmaceutically acceptable carrier or excipient; and, a pharmaceutically acceptable adjuvant.

33. A method of vaccinating a mammal against influenza, said method comprising administering to the mammal intradermally a vaccine comprising:
a therapeutically or prophylactically effective amount of a naked viral core particle, wherein the naked viral core particle is comprised of any combination of one or more of the RNA genome (RNAg), nucleoprotein (NP), RNA polymerase (PA), RNA polymerase 1 (PB 1), RNA polymerase 2 (PB2), non-structural protein 1 (NS1), and non-structural protein 2 (NS2);
a pharmaceutically acceptable carrier or excipient; and,
a pharmaceutically acceptable adjuvant.


SEQUENCE LISTING (50 sequences)

SEQ ID NO: 1 - DNA, 1565 bp, Influenza A virus
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcacagaacttaaactcagtgattatgagggacggttgatccaaaacagcttaacaatagagagaatggtgctctctgcttttgacgaaaggagaaataaatacctggaagaacatcccagtgcggggaaggatcctaagaaaactggaggacctatatacagaagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagggctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccgggacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattgagcttcatcaaagggacgaaggtggtcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccgttatggcagcattcactgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 2 - protein, 498 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRDPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVVPRGKLSTRGVQIASNENMETMESSTLELRSRYWAIRTRSGGNTNQQRASAGQISIQPTFSVQRNLPFDRTTVMAAFTGNTEGRTSDMRTEIIRMMESARPEDVSFQGRGVFELSDEKAASPIVPSFDMSNEGSYFFGDNAEEYDN

SEQ ID NO: 3 - DNA, 1565 bp, Influenza A virus
agcaaaagcagggtagataatcactcactgagtgacatcaacatcatggcgtctcagggcaccaaacgatcttatgaacagatggaaactggtggagaacgccagaatgctactgagatcagagcatctgttggaagaatggttggtggaattgggaggttttatatacagatgtgcactgaactcaaactcagcgactatgaaggaaggctgattcagaacagcataacaatagagagaatggttctctctgcatttgatgaaaggaggaacaaatacctggaagaacatcccagtgcggggaaggacccaaagaaaactggaggtccaatctaccgaagaagagacggaaaatgggtgagagagctgattctgtatgacaaagaggagatcaggagaatttggcgtcaagcgaacaatggagaagatgcaactgctggtctcactcacatgatgatctggcattccaatctaaatgatgccacataccagagaacaagagctctcgtgcgtactgggatggaccctagaatgtgctctctgatgcaaggatcaactctcccgaggagatctggagctgctggtgcggcagtaaagggagtcggaacgatggtgatggaactaattcggatgataaagcgagggattaacgatcggaatttctggagaggtgaaaatgggcgaagaacaagaattgcatatgagagaatgtgcaacatcctcaaagggaaattccaaacagcagcacaaagagcaatgatggatcaggtacgggaaagcagaaatcctgggaatgctgagattgaagatctcatatttctggcacggtctgcactcatcctgagaggatcagtggcccacaagtcctgcttgcctgcttgtgtgtacgggcttgccgtggccagtggatatgactttgagagagaagggtactctctggtcgggattgatcctttccgtctgctgcaaaacagccaggtctttagtctaattagaccaaatgagaatccagcacataaaagtcaattggtgtggatggcatgccattctgcagcatttgaagatctgagagtctcaagcttcatcagagggacaagagtggccccaaggggacaactatctactagaggagttcaaattgcttcaaatgagaacatggaaacaatggactccagcactcttgaactgagaagcagatattgggctataaggaccaggagtggaggaaacaccaaccagcagagagcatctgcaggacaaatcagtgtgcagcctactttctcggtacagagaaatcttcccttcgaaagagcgaccattatggcggcattcacagggaatacagagggcagaacatctgacatgaggactgaaatcataaggatgatggaaagctccagaccagaagatgtgtctttccaggggcggggagtcttcgagctctcggacgaaaaggcaacgaacccgatcgtgccttcctttgacatgagtaatgaaggatcttatttcttcggagacaatgcagaggaatatgacaattgaagaaaaatacccttgtttctact

SEQ ID NO: 4 - protein, 498 aa, Influenza A virus
MASQGTKRSYEQMETGGERQNATEIRASVGRMVGGIGRFYIQMCTELKLSDYEGRLIQNSITIERMVLSAFDERRNKYLEEHPSAGKDPKKTGGPIYRRRDGKWVRELILYDKEEIRRIWRQANNGEDATAGLTHMMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVAPRGQLSTRGVQIASNENMETMDSSTLELRSRYWAIRTRSGGNTNQQRASAGQISVQPTFSVQRNLPFERATIMAAFTGNTEGRTSDMRTEIIRMMESSRPEDVSFQGRGVFELSDEKATNPIVPSFDMSNEGSYFFGDNAEEYDN

SEQ ID NO: 5 - DNA, 1566 bp, Influenza A virus
agcaaaagcagggttaataatcactcaccgagtgacatcaaaatcatggcgtcccaaggcaccaaacggtcttatgaacagatggaaactgatggggatcgccagaatgcaactgagattagggcatccgtcgggaagatgattgatggaattgggagattctacatccaaatgtgcactgaacttaaactcagtgatcatgaagggcggttgatccagaacagcttgacaatagagaaaatggtgctctctgcttttgatgaaagaaggaataaatacctggaagaacaccccagcgcggggaaagatcccaagaaaactggggggcccatatacaggagagtagatggaaaatggatgagggaactcgtcctttatgacaaagaagagataaggcgaatctggcgccaagccaacaatggtgaggatgcgacagctggtctaactcacataatgatctggcattccaatttgaatgatgcaacataccagaggacaagagctcttgttcgaactggaatggatcccagaatgtgctctctgatgcagggctcgactctccctagaaggtccggagctgcaggtgctgcagtcaaaggaatcgggacaatggtgatggaactgatcagaatggtcaaacgggggatcaacgatcgaaatttctggagaggtgagaatgggcggaaaacaagaagtgcttatgagagaatgtgcaacattcttaaaggaaaatttcaaacagctgcacaaagagcaatggtggatcaagtgagagaaagtcggaacccaggaaatgctgagatcgaagatctcatatttttggcaagatctgcattgatattgagagggtcagttgctcacaaatcttgcctacctgcctgtgcgtatggacctgcagtatccagtgggtacgacttcgaaaaagagggatattccttggtgggaatagaccctttcaaactacttcaaaatagccaaatatacagcctaatcagacctaacgagaatccagcacacaagagtcagctggtgtggatggcatgccattctgctgcatttgaagatttaagattgttaagcttcatcagagggacaaaagtatctccgcgggggaaactgtcaactagaggagtacaaattgcttcaaatgagaacatggataatatgggatcgagcactcttgaactgagaagcgggtactgggccataaggaccaggagtggaggaaacactaatcaacagagggcctccgcaggccaaaccagtgtgcaacctacgttttctgtacaaagaaacctcccatttgaaaagtcaaccatcatggcagcattcactggaaatacggagggaaggacttcagacatgagggcagaaatcataagaatgatggaaggtgcaaaaccagaagaagtgtcattccgggggaggggagttttcgagctctcagacgagaaggcaacgaacccgatcgtgccctcttttgatatgagtaatgaaggatcttatttcttcggagacaatgcagaagagtacgacaattaaggaaaaaatacccttgtttctact

SEQ ID NO: 6 - protein, 498 aa, Influenza A virus
MASQGTKRSYEQMETDGDRQNATEIRASVGKMIDGIGRFYIQMCTELKLSDHEGRLIQNSLTIEKMVLSAFDERRNKYLEEHPSAGKDPKKTGGPIYRRVDGKWMRELVLYDKEEIRRIWRQANNGEDATAGLTHIMIWHSNLNDATYQRTRALVRTGMDPRMCSLMQGSTLPRRSGAAGAAVKGIGTMVMELIRMVKRGINDRNFWRGENGRKTRSAYERMCNILKGKFQTAAQRAMVDQVRESRNPGNAEIEDLIFLARSALILRGSVAHKSCLPACAYGPAVSSGYDFEKEGYSLVGIDPFKLLQNSQIYSLIRPNENPAHKSQLVWMACHSAAFEDLRLLSFIRGTKVSPRGKLSTRGVQIASNENMDNMGSSTLELRSGYWAIRTRSGGNTNQQRASAGQTSVQPTFSVQRNLPFEKSTIMAAFTGNTEGRTSDMRAEIIRMMEGAKPEEVSFRGRGVFELSDEKATNPIVPSFDMSNEGSYFFGDNAEEYDN

SEQ ID NO: 7 - DNA, 2341 bp, Influenza A virus
agcgaaagcaggcaaaccatttgaatggatgtcaatccgaccttacttttcttaaaagtgccagcacaaaatgctataagcacaactttcccttataccggagaccctccttacagccatgggacaggaacaggatacaccatggatactgtcaacaggacacatcagtactcagaaaaggcaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactttaaatagaaaccagcctgctgcaacagcattggccaacacaatagaagtgttcagatcaaatggcctcacggccaatgagtctggaaggctcatagacttccttaaggatgtaatggagtcaatgaaaaaagaagaaatggggatcacaactcattttcagagaaagagacgggtgagagacaatatgactaagaaaatgataacacagagaacaataggtaaaaggaaacagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttgaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcattgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacatggaatcaatatgagcaagaaaaagtcttacataaacagaacaggtacatttgaattcacaagttttttctatcgttatgggtttgttgccaatttcagcatggagcttcccagttttggtgtgtctgggagcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgttcctact

SEQ ID NO: 8 - protein, 757 aa, Influenza A virus
MDVNPTLLFLKVPAQNAISTTFPYTGDPPYSHGTGTGYTMDTVNRTHQYSEKARWTTNTETGAPQLNPIDGPLPEDNEPSGYAQTDCVLEAMAFLEESHPGIFENSCIETMEVVQQTRVDKLTQGRQTYDWTLNRNQPAATALANTIEVFRSNGLTANESGRLIDFLKDVMESMKKEEMGITTHFQRKRRVRDNMTKKMITQRTIGKRKQRLNKRSYLIRALTLNTMTKDAERGKLKRRAIATPGMQIRGFVYFVETLARSICEKLEQSGLPVGGNEKKAKLANVVRKMMTNSQDTELSLTITGDNTKWNENQNPRMFLAMITYMTRNQPEWFRNVLSIAPIMFSNKMARLGKGYMFESKSMKLRTQIPAEMLASIDLKYFNDSTRKKIEKIRPLLIEGTASLSPGMMMGMFNMLSTVLGVSILNLGQKRYTKTTYWWDGLQSSDDFALIVNAPNHEGIQAGVDRFYRTCKLHGINMSKKKSYINRTGTFEFTSFFYRYGFVANFSMELPSFGVSGSNESADMSIGVTVIKNNMINNDLGPATAQMALQLFIKDYRYTYRCHRGDTQIQTRRSFEIKKLWEQTRSKAGLLVSDGGPNLYNIRNLHIPEVCLKWELMDEDYQGRLCNPLNPFVSHKEIESMNNAVMMPAHGPAKNMEYDAVATTHSWIPKRNRSILNTSQRGVLEDEQMYQRCCNLFEKFFPSSSYRRPVGISSMVEAMVSRARIDARIDFESGRIKKEEFTEIMKICSTIEELRRQK

SEQ ID NO: 9 - DNA, 2341 bp, Influenza A virus
agcaaaagcaggcaaaccatttgaatggatgtcaatccgactttacttttcttaaaagtgccagcgcaaaatgctataagtaccacattcccttatactggagatcctccatacagccatggaacaggaacaggatacaccatggacacagtcaacagaacacatcaatattcagaaaaggggaaatggacaacgaacacagagactggagcaccccaactcaatccgattgatggaccactacctgaggataatgagccgagtgggtatgcacaaacagattgtgtattggaagcaatggctttccttgaagaatcccacccagggatctttgaaaactcgtgtcttgaaacgatggaagttgttcagcaaacaagagtggataagctgacccaaggtcgccaaacctatgactggacattgaaaagaaaccagccggctgcaaccgctttggccaacactatagaggtcttcagatcgaatggtctaacagccaatgaatcgggaaggctaatagatttcctcaaagacgtgatggaatcaatggataagggagaaatggaaataataacacatttccagagaaagagaagagtgagggacaacatgaccaagaaaatggtcacacaaagaacaatagggaagaaaaaacaaaggctgaacaaaaggagctacctaataagagcactgacactgaacacaatgacaaaagacgcagaaagaggcaaattgaagaggcgggcaattgcaacacccgggatgcaaatcagaggattcgtgtactttgtcgaaacactagcgaggagtatctgtgagaaacttgagcaatctggactccccgtcggagggaatgaaaagaaggctaaattggcaaatgtcgtgaggaagatgatgactaactcacaagatacagagctctcttttacaattactggagacaacaccaaatggaatgagaatcagaaccctcggatgtttctagcaatgataacatacatcacaaggaaccaacctgaatggtttagaaatgtcttaagcattgctcctataatgttctcaaacaagatggcaagattagggaaaggatacatgttcgaaagtaagagcatgaagctacggacacaaataccagcagaaatgcttgcaagcattgacttgaaatacttcaacgaatcaacgagaaagaaaatcgagaaaataagacctctactaatagatggcacagcctcattgagtcctggaatgatgatgggcatgttcaatatgctgagtacagtcttaggagtttcaatcctgaatcttgggcagaagaggtacaccaaaaccacatactggtgggacggactccaatcctctgatgatttcgctctcatagtgaatgcaccaaatcatgagggaatagaagcaggggtggataggttctataggacttgcaaactagttggaatcaatatgaccaagaagaagtcttacataaatcggacaggaacatgtgaattcacaagcttcttctaccgctatgggttcgtagccaacttcagtatggagctgcccagctttggagtgtctgggattaatgaatcggctgacatgagcattggtgttacagtgataaagaacaatatgatggacaacgaccttggaccagcaacagctcagatggctcttcagctattcattaaggactacagatacccataccgatgccacaggggggatacacaaatccaaacgaggagatcattcgagctgaagaagctgtgggagcagacccgctcaaaggcaggactgttggtttcagatggaggaccaaacccatacaatatccggaatctccacattccggaggctggcttgaagtgggaattgatggatgaagactaccagggcagactgtgtaatcctctgaacccgtttgttagtcataaggaaattgagtctgtcaacaatgctgtggtaatgccagctcatggcccagccaagagcatggaatatgatgcagttgcgactacacattcatggattcccaagaggaatcgttccattctcaacaccagccaaagggggattcttgaggatgaacagatgtatcagaagtgctgcaatctattcgagaaattcttccctagcagttcatatcggaggccagttggaatttccagcatggtggaggccatggtgtctagggcccgaattgatgcacgaattgacttcgagtctggaaggattaagaaagaagagtttgctgagatcatgaagatctgttccaccattgaagagctcggacggcaaaaatagtgaatttagcttgtccttcatgaaaaaatgccttgtttctact

SEQ ID NO: 10 - protein, 757 aa, Influenza A virus
MDVNPTLLFLKVPAQNAISTTFPYTGDPPYSHGTGTGYTMDTVNRTHQYSEKGKWTTNTETGAPQLNPIDGPLPEDNEPSGYAQTDCVLEAMAFLEESHPGIFENSCLETMEVVQQTRVDKLTQGRQTYDWTLKRNQPAATALANTIEVFRSNGLTANESGRLIDFLKDVMESMDKGEMEIITHFQRKRRVRDNMTKKMVTQRTIGKKKQRLNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYITRNQPEWFRNVLSIAPIMFSNKMARLGKGYMFESKSMKLRTQIPAEMLASIDLKYFNESTRKKIEKIRPLLIDGTASLSPGMMMGMFNMLSTVLGVSILNLGQKRYTKTTYWWDGLQSSDDFALIVNAPNHEGIEAGVDRFYRTCKLVGINMTKKKSYINRTGTCEFTSFFYRYGFVANFSMELPSFGVSGINESADMSIGVTVIKNNMMDNDLGPATAQMALQLFIKDYRYPYRCHRGDTQIQTRRSFELKKLWEQTRSKAGLLVSDGGPNPYNIRNLHIPEAGLKWELMDEDYQGRLCNPLNPFVSHKEIESVNNAVVMPAHGPAKSMEYDAVATTHSWIPKRNRSILNTSQRGILEDEQMYQKCCNLFEKFFPSSSYRRPVGISSMVEAMVSRARIDARIDFESGRIKKEEFAEIMKICSTIEELGRQK

SEQ ID NO: 11 - DNA, 2341 bp, Influenza A virus
  modified_base (1326)..(1326): a, c, t, g, unknown or other
agcaaaagcaggcaaaccatttgaatggatgtcaatccgaccttacttttcttgaaagttccagcgcaaaatgccataagtactacattcccttatactggagatcctccatacagccatgggacaggaacaggatacaccatggacacagtcaacagaacacatcaatattcagaaaaggggaagtggacaacaaacacggaaactggagcgccccaacttaacccaattgatggaccactacctgaggacaatgaaccaagtggatatgcacaaacagactgcgtcctggaagcaatggctttccttgaggaatcacacccaggaatctttgaaaattcgtgtcttgaaacgatggaagttattcaacaaacaagagtggacaaactgacccaaggtcgtcagacctatgactggacattgaacagaaatcagccggctgcaactgcgctagccaacactatagaggtcttcagatcgaatggactgacagctaatgagtcgggaaggctaatagatttcctcaaggatgtgatagaatcaatggataaagaggagatggaaataacaacacacttccaaagaaaaagaagagtaagagacaacatgaccaagaaaatggtcacacaacgaacaataggaaagaagaagcaaagattgaacaagagaagctatctgataagagcactgacattgaacacaatgactaaagatgcagagagaggtaaattaaaaagaagagcaattgcaacacccggtatgcagatcagagggttcgtgcactttgtcgaaacactagcgagaaatatttgtgagaaacttgaacagtctgggcttccggttggaggtaatgaaaagaaggctaaactagcaaatgttgttagaaaaatgatgactaattcacaagacacagagctctctttcacaattactggagacaacaccaaatggaatgagaatcaaaatcctcgagtgtttctggcgatgataacatacatcacaagaaatcaacctgaatggtttagaaacgtcctgagcattgcacccataatgttctcaaataaaatggctagactagggaaaggttacatgttcgaaagcaagagcatgaagctccgaacacaaataccagcagaaatgctagcaagtattgacctgaaatactttaatgaatcaaccagaaagaaaattgagaaaataaggcctctcctaatagatggcacagtctcattgagtcctggaatgatgatgggcatgttcaacatgctaagtacagtcttaggagtctcaatcctgaatctcgggcaaaagaaatacaccaaaacnacatactggtgggacggactccaatcctctgatgacttcgctctcatagtgaatgcaccaaatcatgagggaatacaagcaggggtgaatagattctacagaacctgcaagctagtcggaatcaatatgagcaaaaagaagtcctacataaataggacagggacatttgaattcacaagctttttctatcgctatggatttgtagccaattttagcatggagctgcccagctttggagtgtctggaattaatgaatcggctgatatgagcattggggtaacagtgataaagaacaatatgataaataatgaccttgggccagcaacagcccaaatggctcttcaactattcatcaaagactacagatacacgtaccggtgccacagaggggacacacaaattcagacaaggagatcattcgagctaaagaagctgtgggagcaaacccgctcaaaggcaggacttttggtgtcggatggaggatcaaacttatacaatatccggaatctccacattccagaagtctgcttgaaatgggagctaatggatgaagactatcaggggaggctttgtaatcccctgaatccatttgtcagtcataaggaaattgagtctgtaaacaatgctgtggtaatgccagctcacggtccagccaagagcatggaatatgatgctgttgctactacacactcctggacccctaagaggaaccgctccattctcaacacaagccaaaggggaattcttgaagatgaacagatgtatcagaagtgttgcaatctatttgagaaattcttccctagcagttcgtacaggagaccagttggaatttccagcatggtggaggccatggtgtctagggctcggattgatgcacggattgacttcgagtctggacggattaagaaagaggagttcgctgagatcatgaagatctgttccaccattgaagagctcagacggcaaaaatagtgaatttagcttgtccttcatgaaaaaatgccttgtttctact

SEQ ID NO: 12 - protein, 757 aa, Influenza A virus
MDVNPTLLFLKVPAQNAISTTFPYTGDPPYSHGTGTGYTMDTVNRTHQYSEKGKWTTNTETGAPQLNPIDGPLPEDNEPSGYAQTDCVLEAMAFLEESHPGIFENSCLETMEVIQQTRVDKLTQGRQTYDWTLNRNQPAATALANTIEVFRSNGLTANESGRLIDFLKDVIESMDKEEMEITTHFQRKRRVRDNMTKKMVTQRTIGKKKQRLNKRSYLIRALTLNTMTKDAERGKLKRRAIATPGMQIRGFVHFVETLARNICEKLEQSGLPVGGNEKKAKLANVVRKMMTNSQDTELSFTITGDNTKWNENQNPRVFLAMITYITRNQPEWFRNVLSIAPIMFSNKMARLGKGYMFESKSMKLRTQIPAEMLASIDLKYFNESTRKKIEKIRPLLIDGTVSLSPGMMMGMFNMLSTVLGVSILNLGQKKYTKTTYWWDGLQSSDDFALIVNAPNHEGIQAGVNRFYRTCKLVGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFELKKLWEQTRSKAGLLVSDGGSNLYNIRNLHIPEVCLKWELMDEDYQGRLCNPLNPFVSHKEIESVNNAVVMPAHGPAKSMEYDAVATTHSWTPKRNRSILNTSQRGILEDEQMYQKCCNLFEKFFPSSSYRRPVGISSMVEAMVSRARIDARIDFESGRIKKEEFAEIMKICSTIEELRRQK

SEQ ID NO: 13 - DNA, 2341 bp, Influenza A virus
agcgaaagcaggtcaattatattcaatatggaaagaataaaagaactaagaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaatgacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaagaatgatgatgttgatcaaagcttgattattgctgctaggaacatagtgagaagagctgcagtatcagcagacccactagcatctttattggagatgtgccacagcacacagattggtggaattaggatggtagacatccttaagcagaacccaacagaagagcaagccgtgggtatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagttagaggtgatctgaatttcgtcaatagggcgaatcagcgactgaatcctatgcatcaacttttaagacattttcagaaggatgcgaaagtgctttttcaaaattggggagttgaacctatcgacaatgtgatgggaatgattgggatattgcccgacatgactccaagcatcgagatgtcaatgagaggagtgagaatcagcaaaatgggtgtagatgagtactccagcacggagagggtagtggtgagcattgaccggttcttgagagtccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtgagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactacaacaaggccacgaagagactcacagttctcggaaaggatgctggcactttaaccgaagacccagatgaaggcacagctggagtggagtccgctgttctgaggggattcctcattctgggcaaagaagacaggagatatgggccagcattaagcatcaatgaactgagcaaccttgcgaaaggagagaaggctaatgtgctaattgggcaaggagacgtggtgttggtaatgaaacgaaaacgggactctagcatacttactgacagccagacagcgaccaaaagaattcggatggccatcaattagtgtcgaatagtttaaaaacgaccttgtttctact

SEQ ID NO: 14 - protein, 759 aa, Influenza A virus
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPMTNTVHYPKIYKTYFERVERLKHGTFGPVHFRNQVKIRRRVDINPGHADLSAKEAQDVIMEVVFPNEVGARILTSESQLTITKEKKEELQDCKISPLMVAYMLERELVRKTRFLPVAGGTSSVYIEVLHLTQGTCWEQMYTPGGEVKNDDVDQSLIIAARNIVRRAAVSADPLASLLEMCHSTQIGGIRMVDILKQNPTEEQAVGICKAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGVEPIDNVMGMIGILPDMTPSIEMSMRGVRISKMGVDEYSSTERVVVSIDRFLRVRDQRGNVLLSPEEVSETQGTEKLTITYSSSMMWEINGPESVLVNTYQWIIRNWETVKIQWSQNPTMLYNKMEFEPFQSLVPKAIRGQYSGFVRTLFQQMRDVLGTFDTAQIIKLLPFAAAPPKQSRMQFSSFTVNVRGSGMRILVRGNSPVFNYNKATKRLTVLGKDAGTLTEDPDEGTAGVESAVLRGFLILGKEDRRYGPALSINELSNLAKGEKANVLIGQGDVVLVMKRKRDSSILTDSQTATKRIRMAIN

SEQ ID NO: 15 - DNA, 2341 bp, Influenza A virus
agcaaaagcaggtcaattatattcaatatggaaagaataaaagaactaagagatctaatgtcgcagtcccgcactcgcgagatactaacaaaaaccactgtggatcatatggccataatcaagaaatacacatcaggaagacaagagaagaaccctgctctcagaatgaaatggatgatggcaatgaaatatccaatcacagcagacaagagaataatggagatgattcctgaaaggaatgagcaaggacaaacgctttggagcaagacaaatgatgctgggtcggacagagtgatggtgtctcccctagctgtaacttggtggaacaggaatgggccgacaacaagtacagtccattatccaaaggtttacaaaacatactttgagaaggttgaaaggttaaaacatggaaccttcggtcccgttcatttccgaaaccaagttaaaatacgtcgccgggtggatataaacccgggccatgcagatctcagtgctaaagaagcacaagatgttatcatggaggtcgttttcccaaatgaagtgggagctagaatattgacatcagagtcgcaattgacaataacaaaagagaagaaagaagagctccaggattgtaaaattgctcctttaatggtggcatacatgttggaaagagaactggtccgcaaaaccagatttctaccggtagcaggcggaacaagcagtgtgtacattgaggtattgcatttgactcaagggacctgttgggaacagatgtacactcccggcggagaagtaagaaatgatgatgttgaccagagtttgatcatcgctgccagaaacattgttaggagagcaacagtatcagcggacccactggcatcactcttggagatgtgtcacagcacacaaattgggggaataaggatggtggacatccttaggcaaaacccaactgaggagcaagctgtggatatatgcaaagcagcaatgggtttgaggatcagttcatcctttagctttggaggcttcactttcaaaagaacaaatggatcatccgtcaagaaggaagaggaagtgcttacaggcaacctccaaacattgaaaataaaagtacatgaggggtatgaagaattcacaatggttgggcggagagcaacagctatcctgaggaaagcaactagaaggctgattcagttgatagtaagtggaagagatgaacaatcaatcgctgaagcgatcattgtagcaatggtgttctcacaggaggattgcatgataaaggcagtccgaggcgatctgaatttcgtgaacagagcaaaccaaagattgaaccccatgcatcaactcctgaggcacttccaaaaagatgcaaaagtgctgtttcagaactggggaattgaacctattgacaatgtcatggggatgatcggaatattacctgacatgactccaagcgcagagatgtcactgagaggagtgagagttagtaagatgggagtagatgaatattccagcacggagagagtggtggtgagtattgaccgtttcttgagggtccgagatcagcaggggaacgtactcttatctcctgaagaggttagtgaaacacagggaacagagaagttgacaataacatattcatcctcaatgatgtgggaaatcaacggtcctgagtcagtgcttgttaacacttatcaatggatcatcaggaattgggagactgtaaagattcaatggtctcaagatcccacaatgctgtacaataagatggagtttgaatcgttccaatccttggtgccaaaggctgccagaagccaatatagtggatttgtgagaacactattccaacagatgcgtgatgttttggggacatttgatactgtccaaataatcaagctgctaccatttgcagcagccccaccggagccgagcagaatgcagttttcttctctaactgtgaatgtgagaggctcaggaatgagaatactcgtgaggggtaactcccccgtgttcaactacaacaaggcaaccaaaaggcttacagtcctcggaaaggacgcaggtgcattaacagaagatccagacgagggaacagccggggtggaatctgcagtattgaggggattcctaattctaggcagagaggacaaaagatatggacccgcattgagcatcaatgaactgagcaatcttgcaaaaggggagaaggctaatgtattgataatgcaaggagacgtggtgttggtaatgaaacggaaacgggactttagcatacttactgacagccagacagcgaccaaaagaattcggatggccatcaattagtgttgaatagtttaaaaacgaccttgtttctact

SEQ ID NO: 16 - protein, 759 aa, Influenza A virus
MERIKELRDLMSQSRTREILTKTTVDHMAIIKKYTSGRQEKNPALRMKWMMAMKYPITADKRIMEMIPERNEQGQTLWSKTNDAGSDRVMVSPLAVTWWNRNGPTTSTVHYPKVYKTYFEKVERLKHGTFGPVHFRNQVKIRRRVDINPGHADLSAKEAQDVIMEVVFPNEVGARILTSESQLTITKEKKEELQDCKIAPLMVAYMLERELVRKTRFLPVAGGTSSVYIEVLHLTQGTCWEQMYTPGGEVRNDDVDQSLIIAARNIVRRATVSADPLASLLEMCHSTQIGGIRMVDILRQNPTEEQAVDICKAAMGLRISSSFSFGGFTFKRTNGSSVKKEEEVLTGNLQTLKIKVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGIEPIDNVMGMIGILPDMTPSAEMSLRGVRVSKMGVDEYSSTERVVVSIDRFLRVRDQQGNVLLSPEEVSETQGTEKLTITYSSSMMWEINGPESVLVNTYQWIIRNWETVKIQWSQDPTMLYNKMEFESFQSLVPKAARSQYSGFVRTLFQQMRDVLGTFDTVQIIKLLPFAAAPPEPSRMQFSSLTVNVRGSGMRILVRGNSPVFNYNKATKRLTVLGKDAGALTEDPDEGTAGVESAVLRGFLILGREDKRYGPALSINELSNLAKGEKANVLIMQGDVVLVMKRKRDFSILTDSQTATKRIRMAIN

SEQ ID NO: 17 - DNA, 2341 bp, Influenza A virus
agcaaaagcaggtcaattatattcagtatggaaagaataaaagaactacggaacctgatgtcgcagtctcgcactcgcgagatactgacaaaaaccacagtggaccatatggccataattaagaagtacacatcggggagacaggaaaagaacccgtcacttaggatgaaatggatgatggcaatgaaatacccaatcactgctgacaaaaggataacagaaatggttccggagagaaatgaacaaggacaaactctatggagtaaaatgagtgatgctggatcagatcgagtgatggtatcacctttggctgtaacatggtggaatagaaatggacccgtggcaagtacggtccattacccaaaagtatacaagacttattttgacaaagtcgaaaggttaaaacatggaacctttggccctgttcattttagaaatcaagtcaagatacgcagaagagtagacataaaccctggtcatgcagacctcagtgccaaagaggcacaagatgtaattatggaagttgtttttcccaatgaagtgggagccaggatactaacatcagaatcgcaattaacaataactaaagagaaaaaagaagaactccgagattgcaaaatttctcccttgatggttgcatacatgttagagagagaacttgtccgaaaaacaagatttctcccagttgctggcggaacaagcagtatatacattgaagtcttacatttgactcaaggaacgtgttgggaacaaatgtacactccaggtggagaagtgaggaatgacgatgttgaccaaagcctaattattgcggccaggaacatagtaagaagagctgcagtatcagcagatccactagcatctttattggagatgtgccacagcacacaaattggcgggacaaggatggtggacattcttagacagaacccgactgaagaacaagctgtggatatatgcaaggctgcaatgggattgagaatcagctcatccttcagctttggtgggtttacatttaaaagaacaagcgggtcatcagtcaaaaaagaggaagaagtgcttacaggcaatctccaaacattgaagataagagtacatgaggggtatgaggagttcacaatggtggggaaaagagcaacagctatactcagaaaagcaaccagaagattggttcagctcatagtgagtggaagagacgaacagtcaatagccgaagcaataatcgtggccatggtgttttcacaagaggattgcatgataaaagcagttagaggtgacctgaatttcgtcaacagagcaaatcaacggttgaaccccatgcatcagcttttaaggcattttcagaaagatgcgaaagtgctttttcaaaattggggaattgaacacatcgacagtgtgatgggaatggttggagtattaccagatatgactccaagcacagagatgtcaatgagaggaataagagtcagcaaaatgggtgtggatgaatactccagtacagagagggtggtggttagcattgatcggtttttgagagttcgagaccaacgcgggaatgtattattgtctcctgaggaggtcagtgaaacacagggaactgaaagattgacaataacatattcatcgtcgatgatgtgggagattaacggtcctgagtcggttttggtcaatacctatcaatggatcatcagaaattgggaagctgtcaaaattcaatggtctcagaatcctgcaatgttgtacaacaaaatggaatttgaaccatttcaatctttagtccccaaggccattagaagccaatacagtgggtttgtcagaactctattccaacaaatgagagacgtacttgggacatttgacaccacccagataataaagcttctcccttttgcagccgctccaccaaagcaaagcagaatgcagttctcttcactgactgtaaatgtgaggggatcagggatgagaatacttgtaaggggcaattctcctgtattcaactacaacaagaccactaaaagactaacaattctcggaaaagatgccggcactttaattgaagacccagatgaaagcacatccggagtggagtccgccgtcttgagagggtttctcattataggtaaggaagacagaagatacggaccagcattaagcatcaatgaactgagtaaccttgcaaaaggggaaaaggctaatgtgctaatcgggcaaggagacgtggtgttggtaatgaaacgaaaacgggactctagcatacttactgacagccagacagcgaccaaaagaattcggatggccatcaattaatgttgaatagtttaaaaacgaccttgtttctact

SEQ ID NO: 18 - protein, 759 aa, Influenza A virus
MERIKELRNLMSQSRTREILTKTTVDHMAIIKKYTSGRQEKNPSLRMKWMMAMKYPITADKRITEMVPERNEQGQTLWSKMSDAGSDRVMVSPLAVTWWNRNGPVASTVHYPKVYKTYFDKVERLKHGTFGPVHFRNQVKIRRRVDINPGHADLSAKEAQDVIMEVVFPNEVGARILTSESQLTITKEKKEELRDCKISPLMVAYMLERELVRKTRFLPVAGGTSSIYIEVLHLTQGTCWEQMYTPGGEVRNDDVDQSLIIAARNIVRRAAVSADPLASLLEMCHSTQIGGTRMVDILRQNPTEEQAVDICKAAMGLRISSSFSFGGFTFKRTSGSSVKKEEEVLTGNLQTLKIRVHEGYEEFTMVGKRATAILRKATRRLVQLIVSGRDEQSIAEAIIVAMVFSQEDCMIKAVRGDLNFVNRANQRLNPMHQLLRHFQKDAKVLFQNWGIEHIDSVMGMVGVLPDMTPSTEMSMRGIRVSKMGVDEYSSTERVVVSIDRFLRVRDQRGNVLLSPEEVSETQGTERLTITYSSSMMWEINGPESVLVNTYQWIIRNWEAVKIQWSQNPAMLYNKMEFEPFQSLVPKAIRSQYSGFVRTLFQQMRDVLGTFDTTQIIKLLPFAAAPPKQSRMQFSSLTVNVRGSGMRILVRGNSPVFNYNKTTKRLTILGKDAGTLIEDPDESTSGVESAVLRGFLIIGKEDRRYGPALSINELSNLAKGEKANVLIGQGDVVLVMKRKRDSSILTDSQTATKRIRMAIN

SEQ ID NO: 19 - DNA, 2233 bp, Influenza A virus
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagatttccacttcatcaatgagcaaggcgagtcaataatcgtagaacttggtgatcctaatgcacttttgaagcacagatttgaaataatcgagggaagagatcgcacaatggcctggacagtagtaaacagtatttgcaacactacaggggctgagaaaccaaagtttctaccagatttgtatgattacaaggaaaatagattcatcgaaattggagtaacaaggagagaagttcacatatactatctggaaaaggccaataaaattaaatctgagaaaacacacatccacattttctcgttcactggggaagaaatggccacaaaggccgactacactctcgatgaagaaagcagggctaggatcaaaaccaggctattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaaaaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagttcaacaaggcatgcgaactgacagattcaagctggatagagcttgatgagattggagaagatgtggctccaattgaacacattgcaagcatgagaaggaattatttcacatcagaggtgtctcactgcagagccacagaatacataatgaagggggtgtacatcaatactgccttacttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacacaaatgggagaagtactgtgttcttgagataggagatatgcttctaagaagtgccataggccaggtttcaaggcccatgttcttgtatgtgaggacaaatggaacctcaaaaattaaaatgaaatggggaatggagatgaggcgttgtctcctccagtcacttcaacaaattgagagtatgattgaagctgagtcctctgtcaaagagaaagacatgaccaaagagttctttgagaacaaatcagaaacatggcccattggagagtctcccaaaggagtggaggaaagttccattgggaaggtctgcaggactttattagcaaagtcggtatttaacagcttgtatgcatctccacaactagaaggattttcagctgaatcaagaaaactgcttcttatcgttcaggctcttagggacaatctggaacctgggacctttgatcttggggggctatatgaagcaattgaggagtgcctaattaatgatccctgggttttgcttaatgcttcttggttcaactccttccttacacatgcattgagttagttgtggcagtgctactatttgctatccatactgtccaaaaaagtaccttgtttctact

SEQ ID NO: 20 - protein, 716 aa, Influenza A virus
MEDFVRQCFNPMIVELAEKTMKEYGEDLKIETNKFAAICTHLEVCFMYSDFHFINEQGESIIVELGDPNALLKHRFEIIEGRDRTMAWTVVNSICNTTGAEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRKLADQSLPPNFSSLENFRAYVDGFEPNGYIEGKLSQMSKEVNARIEPFLKTTPRPLRLPNGPPCSQRSKFLLMDALKLSIEDPSHEGEGIPLYDAIKCMRTFFGWKEPNVVKPHEKGINPNYLLSWKQVLAELQDIENEEKIPKTKNMKKTSQLKWALGENMAPEKVDFDDCKDVGDLKQYDSDEPELRSLASWIQNEFNKACELTDSSWIELDEIGEDVAPIEHIASMRRNYFTSEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRSAIGQVSRPMFLYVRTNGTSKIKMKWGMEMRRCLLQSLQQIESMIEAESSVKEKDMTKEFFENKSETWPIGESPKGVEESSIGKVCRTLLAKSVFNSLYASPQLEGFSAESRKLLLIVQALRDNLEPGTFDLGGLYEAIEECLINDPWVLLNASWFNSFLTHALS

SEQ ID NO: 21 - DNA, 2233 bp, Influenza A virus
agcaaaagcaggtactgatccaaaatggaagactttgtgcgacaatgcttcaatccaatgattgtcgagcttgcggaaaaggcaatgaaagaatatggggaagatccgaaaatcgaaacgaacaaatttgccgcaatatgcacgcacttagaagtctgtttcatgtattcagatttccactttattgatgaacggggcgaatcaacaattatagaatctggcgatcccaatgcattattgaaacaccggtttgaaataatcgaagggagggaccgaacaatggcctggacagtggtgaatagtatctgcaacaccacaggagttgagaagcctaaatttctcccagatttgtatgactacaaggagaaccgatttattgaaattggagtgacacggagggaagttcacacatactatctagaaaaagccaacaagataaaatctgagaagacacacattcacatattctcattcactggagaggaaatggccaccaaagcggactacacccttgatgaagaaagcagggcccgaatcaaaaccaggctgttcactataaggcaggaaatggccagtaggggtttatgggattcctttcgtcagtccgagagaggcgaagagacagttgaagaaagatttgaaatcacagggactatgtgcaggcttgccgaccaaagtctcccacctaatttctccagccttgaaaaatttagagcctatgtggatggattcgaaccgaacggctgcattgagggcaagctttctcaaatgtcgaaagaagtaaacgccagaattgagccatttctgaagacaacaccacgccctcttagattacctgatgggcctccctgctctcagcggtcgaagtttttgctgatggatgcccttaaattaagcatcgaagacccgagtcatgagggggaggggataccgctatatgatgcaatcaaatgcatgaaaacatttttcggctggaaagagcccaacattgtaaaaccacatgaaaaaggcataaaccccaattacctcctggcttggaagcaggtgctggcagagctccaagatattgaaaacgaggagaaaattccaaagacaaagaacatgaggaaaacaagccaattgaagtgggcacttggtgagaatatggcaccagagaaagtagactttgaggattgcaaagatgttagcgatctaaggcagtatgacagtgatgaaccaaagcctagatcactagcaagctggatccagagtgaattcaacaaggcatgcgaattgacagattcaagttggattgaacttgatgaaataggggaagacgttgctccaattgagcacattgcaagtatgagaaggaactatttcacagcggaagtatcccattgcagggctactgaatacataatgaagggagtgtacataaacacagctttgttgaatgcatcctgtgcagccatggatgacttccaactgatcccaatgataagcaaatgcagaaccaaagaaggaagacggaaaactaacctgtatggattccttataaaaggaagatcccatttgagaaatgacaccgatgtggtaaactttgtgagtatggaattctctcttactgatccgaggctggagccacacagatgggaaaagtactgcgttcttcggataggagacatgctcttacggactgaaataggccaagtgtcaaggcccatgtttctttatgtgagaaccaatggaacctccaagatcaagatgaaatggggcatggaaatgaggcgatgcccttttcaatcccttcaacagattgagagcatgattgaggccgagtcttctgtcaaagaaaaagacatgactaaagaattctttgaaaacaaatcagaaacatggccaattggagaatcacccaagggagtggaggaaggctccatcgggaaggtgtgcagaaccttactggctaaatctgttttcaacagtctatatgcatctccacaactcgaggggttttcagctgaatcaagaaaattgcttctcattgttcaggcacttagggacaacctggaacctggaaccttcgatcttggggggctatatgaagcaattgaggagtgcctgattaatgatccctgggttttgcttaatgcatcttggttcaactccttcctcacacatgcactaagatagttgtggcaatgctactatttgctatccatactgtccaaaaaagtaccttgtttctact

SEQ ID NO: 22 - protein, 716 aa, Influenza A virus
MEDFVRQCFNPMIVELAEKAMKEYGEDPKIETNKFAAICTHLEVCFMYSDFHFIDERGESTIIESGDPNALLKHRFEIIEGRDRTMAWTVVNSICNTTGVEKPKFLPDLYDYKENRFIEIGVTRREVHTYYLEKANKIKSEKTHIHIFSFTGEEMATKADYTLDEESRARIKTRLFTIRQEMASRGLWDSFRQSERGEETVEERFEITGTMCRLADQSLPPNFSSLEKFRAYVDGFEPNGCIEGKLSQMSKEVNARIEPFLKTTPRPLRLPDGPPCSQRSKFLLMDALKLSIEDPSHEGEGIPLYDAIKCMKTFFGWKEPNIVKPHEKGINPNYLLAWKQVLAELQDIENEEKIPKTKNMRKTSQLKWALGENMAPEKVDFEDCKDVSDLRQYDSDEPKPRSLASWIQSEFNKACELTDSSWIELDEIGEDVAPIEHIASMRRNYFTAEVSHCRATEYIMKGVYINTALLNASCAAMDDFQLIPMISKCRTKEGRRKTNLYGFLIKGRSHLRNDTDVVNFVSMEFSLTDPRLEPHRWEKYCVLRIGDMLLRTEIGQVSRPMFLYVRTNGTSKIKMKWGMEMRRCPFQSLQQIESMIEAESSVKEKDMTKEFFENKSETWPIGESPKGVEEGSIGKVCRTLLAKSVFNSLYASPQLEGFSAESRKLLLIVQALRDNLEPGTFDLGGLYEAIEECLINDPWVLLNASWFNSFLTHALR

SEQ ID NO: 23 - DNA, 2233 bp, Influenza A virus
agcaaaagcaggtactgattcgaaatggaagattttgtgcgacaatgcttcaatccgatgattgtcgaacttgcggaaaaggcaatgaaagagtatggagaagatctgaaaatcgaaacaaacaaatttgcagcaatatgcactcacttggaagtatgcttcatgtattcagattttcatttcatcaatgagcaaggcgagtcaataatggtagagcttgatgatccaaatgcacttttgaagcacagatttgaaataatagagggaagagatcgcacaatggcctggacagtagtaaacagtatttgcaacaccacaggagctgagaaaccgaagtttctgccagatttgtatgattacaaggagaatagattcatcgagattggagtgacaaggagagaagtccacatatactatcttgaaaaggccaataaaattaaatctgagaatacacacatccacattttctcattcactggggaagaaatggccacaaaggccgactacactctcgatgaggaaagcagggctaggatcaaaaccagactattcaccataagacaagaaatggccaacagaggcctctgggattcctttcgtcagtccgaaagaggcgaagaaacaattgaagaaagatttgaaatcacagggacaatgcgcaggcttgccgaccaaagtctcccgccgaacttctcctgccttgagaattttagagcctatgtggatggattcgaaccgaacggctacattgagggcaagctttctcaaatgtccaaagaagtaaatgcaaaaattgaaccttttctgaaaacaacaccaagaccaattagacttccggatgggcctccttgttttcagcggtccaaattcctgctgatggatgctttaaaattaagcattgaggacccaagtcacgaaggggagggaataccactatatgatgcgatcaagtgcatgagaacattctttggatggaaagaaccctatattgttaaaccacacgaaaagggaataaatccaaattatctgctgtcatggaagcaagtactggcggaactgcaggacattgagaatgaggagaagattccaagaactaaaaacatgaagaaaacgagtcagctaaagtgggcacttggtgagaacatggcaccagagaaggtagactttgacaactgtagagacataagcgatttgaagcaatatgatagtgacgaacctgaattaaggtcactttcaagctggatccagaatgagttcaacaaggcatgcgagctgaccgattcaatctggatagagctcgatgagattggagaagacgtggctccaattgaacacattgcaagcatgagaaggaattacttcacagcagaggtgtcccattgcagagccacagaatatataatgaagggggtatacattaatactgccttgcttaatgcatcctgtgcagcaatggacgatttccaactaattcccatgataagcaagtgtagaactaaagagggaaggcgaaagaccaatttatatggtttcatcataaaaggaagatctcacttaaggaatgacaccgacgtggtaaactttgtgagcatggagttttctctcactgacccgagacttgagccacacaaatgggagaagtactgtgtccttgagataggagatatgctactaagaagtgccataggccagatgtcaaggcctatgttcttgtatgtgaggacaaatggaacatcaaagattaaaatgaaatggggaatggagatgaggccttgcctccttcagtcactacaacaaatcgagagtatggttgaagccgagtcctctgtcaaagagaaagacatgaccaaagagttttttgagaataaatcagaaacatggcccattggggagtcccccaaaggagtggaagaaggttccattgggaaggtctgcaggactttattagccaagtcggtattcaatagcctgtatgcatccccacaattagaaggattttcagctgaatcaagaaaactgcttcttgtcgttcaggctcttagggacaatcttgaacctggaacctttgatcttggggggctatatgaagcaattgaggagtgcctgattaatgatccctgggttttgcttaatgcgtcttggttcaactccttcctaacacatgcattaagatagttgtggcaatgctactatttgctatccatactgtccaaaaaagtaccttgtttctact

SEQ ID NO: 24 - protein, 716 aa, Influenza A virus
MEDFVRQCFNPMIVELAEKAMKEYGEDLKIETNKFAAICTHLEVCFMYSDFHFINEQGESIMVELDDPNALLKHRFEIIEGRDRTMAWTVVNSICNTTGAEKPKFLPDLYDYKENRFIEIGVTRREVHIYYLEKANKIKSENTHIHIFSFTGEEMATKADYTLDEESRARIKTRLFTIRQEMANRGLWDSFRQSERGEETIEERFEITGTMRRLADQSLPPNFSCLENFRAYVDGFEPNGYIEGKLSQMSKEVNAKIEPFLKTTPRPIRLPDGPPCFQRSKFLLMDALKLSIEDPSHEGEGIPLYDAIKCMRTFFGWKEPYIVKPHEKGINPNYLLSWKQVLAELQDIENEEKIPRTKNMKKTSQLKWALGENMAPEKVDFDNCRDISDLKQYDSDEPELRSLSSWIQNEFNKACELTDSIWIELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRSAIGQMSRPMFLYVRTNGTSKIKMKWGMEMRPCLLQSLQQIESMVEAESSVKEKDMTKEFFENKSETWPIGESPKGVEEGSIGKVCRTLLAKSVFNSLYASPQLEGFSAESRKLLLVVQALRDNLEPGTFDLGGLYEAIEECLINDPWVLLNASWFNSFLTHALR

SEQ ID NO: 25 - DNA, 890 bp, Influenza A virus
agcaaaagcagggtgacaaagacataatggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcactcttggtctggacatcgagacagccacacgtgctggaaagcagatagtggagcggattctgaaagaagaatccgatgaggcacttaaaatgaccatggcctctgtacctgcgtcgcgttacctaaccgacatgactcttgaggaaatgtcaagggaatggtccatgctcatacccaagcagaaagtggcaggccctctttgtatcagaatggaccaggcgatcatggataaaaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaaggtaacagagaatagttttgagcaaataacatttatgcaagccttacatctattgcttgaagtggagcaagagataagaactttctcatttcagcttatttaataataaaaaacacccttgtttctact

SEQ ID NO: 26 - protein, 121 aa, Influenza A virus
MDPNTVSSFQDILLRMSKMQLESSSEDLNGMITQFESLKLYRDSLGEAVMRMGDLHSLQNRNEKWREQLGQKFEEIRWLIEEVRHKLKVTENSFEQITFMQALHLLLEVEQEIRTFSFQLI

SEQ ID NO: 27 - DNA, 890 bp, Influenza A virus
agcaaaagcagggtgacaaagacataatggattccaacactgtgtcaagctttcaggtagactgctttctttggcatgtccgcaaacgatttgcagaccaagaactgggtgatgccccattccttgaccggcttcgccgagatcagaagtccctaagaggaagaggcagcactcttggtctggacatcagaactgccactcgtgaaggaaagcatatagtggagcggattctggaggaagaatctgacgaggcacttaaaatgactatcgcttcagtgcctgcttcacgctacctaactgaaatgactcttgaggaaatgtcaagggattggttaatgctcattcccaagcagaaagtgacagggcccctttgcattagaatggaccaggcagtaatgggtaaaaccatcatattgaaagcaaactttagtgtgatttttaatcgacttgaagctctgatactacttagagcgtttacagatgaaggagcaatagtgggcgaaatctcaccattaccttcccttccaggacatactgacgaggatgtcaaaaatgcaattggggtcctcatcggaggacttgaatggaatgataacacagttcgagtctctgaaactctacagagattcacttggagaagcagtgatgagaatgggagatctccactccctccaaaacagaaacggaaagtggagagaacaattgagccagaagtttgaagagataagatggttaattgaagaaatgcgacataggttaagaattacagagaatagctttgagcaaataacctttatgcaagccttacaactattgcttgaagtggagcaagagataagaactttctcgtttcagcttatttaatgataaaaaacacccttgtttctact

SEQ ID NO: 28 - protein, 121 aa, Influenza A virus
MDSNTVSSFQDILTRMSKMQLGSSSEDLNGMITQFESLKLYRDSLGEAVMRMGDLHSLQNRNGKWREQLSQKFEEIRWLIEEMRHRLRITENSFEQITFMQALQLLLEVEQEIRTFSFQLI

SEQ ID NO: 29 - DNA, 890 bp, Influenza A virus
agcaaaagcagggtgacaaagacataatggattccaacactgtgtcaagtttccaggtagattgctttctttggcatatccggaaacaagttgtagaccaagaactgagtgatgccccattccttgatcggcttcgccgagatcagaggtccctaaggggaagaggcaatactctcggtctagacatcaaagcagccacccatgttggaaagcaaattgtagaaaagattctgaaagaagaatctgatgaggcacttaaaatgaccatggtctccacacctgcttcgcgatacataactgacatgactattgaggaattgtcaagaaactggttcatgctaatgcccaagcagaaagtggaaggacctctttgcatcagaatggaccaggcaatcatggagaaaaacatcatgttgaaagcgaatttcagtgtgatttttgaccgactagagaccatagtattactaagggctttcaccgaagagggagcaattgttggcgaaatctcaccattgccttcttttccaggacatactattgaggatgtcaaaaatgcaattggggtcctcatcggaggacttgaatggaatgataacacagttcgagtctctaaaaatctacagagattcgcttggagaagcagtaatgagaatgggggacctccacttactccaaaacagaaacggaaaatggcgagaacagctaggtcaaaagtttgaagagataagatggctgattgaagaagtgagacacagactaaaaacaactgaaaatagctttgaacaaataacattcatgcaagcattacaactgctgtttgaagtggaacaggagataagaactttctcatttcagcttatttaatgataaaaaacacccttgtttctact

SEQ ID NO: 30 - protein, 121 aa, Influenza A virus
MDSNTVSSFQDILLRMSKMQLGSSSEDLNGMITQFESLKIYRDSLGEAVMRMGDLHLLQNRNGKWREQLGQKFEEIRWLIEEVRHRLKTTENSFEQITFMQALQLLFEVEQEIRTFSFQLI

SEQ ID NO: 31 - DNA, 1027 bp, Influenza A virus
agcgaaagcaggtagatattgaaagatgagtcttctaaccgaggtcgaaacgtacgttctctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaacccactaatcagacatgagaacagaatggttttagccagcactacagctaaggctatggagcaaatggctggatcgagtgagcaagcagcagaggccatggaggttgctagtcaggctaggcaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcgctattgccgcaaatatcattgggatcttgcacttgatattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 32 - protein, 252 aa, Influenza A virus
MSLLTEVETYVLSIIPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQMVTTTNPLIRHENRMVLASTTAKAMEQMAGSSEQAAEAMEVASQARQMVQAMRTIGTHPSSSAGLKNDLLENLQAYQKRMGVQMQRFK

SEQ ID NO: 33 - DNA, 1027 bp, Influenza A virus
agcaaaagcaggtagatattgaaagatgagccttctaaccgaggtcgaaacgtacgttctctctatcgtcccgtcaggccccctcaaagccgagatcgcacagagacttgaagatgtctttgctgggaagaacacagatcttgaggctctcatggaatggctaaagacaagaccaatcctgtcacctctgactaaggggattttgggatttgtattcacgctcaccgtgccaagtgagcgaggactgcagcgtagacgctttgtccaaaatgccctcaatgggaatggggatccaaataacatggacagagcagttaaactgtatagaaagcttaagagggagataacattccatggggccaaagaagtagcgctcagttattctgctggtgcacttgccagttgcatgggcctcatatacaacaggatgggggctgtgaccactgaagtggcctttgccgtggtatgtgcaacctgtgaacagattgctgactcccagcataggtctcacaggcaaatggtgacaacaaccaatccactaataagacatgagaacagaatggttctggccagcactacagctaaggctatggagcaaatggctggatcgagtgagcaagcagcagaggccatggaggttgctagtcaggccaggcaaatggtgcaggcaatgagagccattgggactcctcctagctccagtgctggtctaaaagatgatcttcttgaaaatttgcaggcctatcagaaacgaatgggggtgcagatgcaacgattcaagtgacccccttgttgttgctgcgagtatcattgggatcttgcactttatattgtggattcttgatcgtctttttttcaaatgcatttatcgcttctttaaacacggtctgaaaagagggccttctacggaaggagtacctgagtctatgagggaagaatatcgaaaggaacagcagagtgctgtggatgctgacgatagtcattttgtcagcatagagctggagtaaaaaactaccttgtttctact

SEQ ID NO: 34 - protein, 252 aa, Influenza A virus
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSAGALASCMGLIYNRMGAVTTEVAFAVVCATCEQIADSQHRSHRQMVTTTNPLIRHENRMVLASTTAKAMEQMAGSSEQAAEAMEVASQARQMVQAMRAIGTPPSSSAGLKDDLLENLQAYQKRMGVQMQRFK

SEQ ID NO: 35 - DNA, 1027 bp, Influenza A virus
agcaaaagcaggtagatattgaaaaatgagtcttctaaccgaggtcgaaacgtacgttctctctatcgtcccgtcaggccccctcaaagccgagatcgcgcagagacttgaggatgtctttgcaggaaagaacaccgatctcgaggctctcatggaatggctaaagacaagaccaatcctgtcacctctgactaaagggattttaggatttgtgttcacgctcaccgtgcccagtgagcgaggactgcagcgtagacgctttgtccagaatgccttaaatggaaatggagatccaaacaatatggatagggcagttaagctatacaagaagctgaaaagagaaataacattccatggggctaaggaggtcgcactcagctactcaaccggtgcacttgccagttgtatgggtctcatatacaacaggatgggaacggtgaccacagaagtggcttttggcctagtgtgtgccacttgtgagcagattgcagattcacagcatcggtctcacagacagatggcaactaccaccaacccactaatcaggcatgagaacagaatggtgctggccagcactacagctaaggctatggagcagatggctggatcgagtgagcaggcagcggaagccatggaggttgctagtcaggctaggcagatggtgcaggcaatgaggacaattgggactcatcctagctccagtgccggtctgaaagataatcttcttgaaaatttgcaggcctaccaaaaacgaatgggagtgcaaatgcagcgattcaagtgatcctcttgttgttgccgcaagtatcattgggatactgcacttgatattgtggattcttgatcgtcttttcttcaaatgcatttatcgtcgccttaaatacggtttgaaaagagggccttctacggaaggggtacctgagtctatgagggaagagtatcggcaggaacagcagagtgctgtggatgttgacgatggtcattttgtcaacatagagctggagtaaaaaactaccttgtttctact

SEQ ID NO: 36 - protein, 252 aa, Influenza A virus
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYKKLKREITFHGAKEVALSYSTGALASCMGLIYNRMGTVTTEVAFGLVCATCEQIADSQHRSHRQMATTTNPLIRHENRMVLASTTAKAMEQMAGSSEQAAEAMEVASQARQMVQAMRTIGTHPSSSAGLKDNLLENLQAYQKRMGVQMQRFK

SEQ ID NO: 37 - DNA, 890 bp, Influenza A virus; identical to SEQ ID NO: 25

SEQ ID NO: 38 - protein, 230 aa, Influenza A virus
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSREWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 39 - DNA, 890 bp, Influenza A virus; identical to SEQ ID NO: 29

SEQ ID NO: 40 - protein, 230 aa, Influenza A virus
MDSNTVSSFQVDCFLWHIRKQVVDQELSDAPFLDRLRRDQRSLRGRGNTLGLDIKAATHVGKQIVEKILKEESDEALKMTMVSTPASRYITDMTIEELSRNWFMLMPKQKVEGPLCIRMDQAIMEKNIMLKANFSVIFDRLETIVLLRAFTEEGAIVGEISPLPSFPGHTIEDVKNAIGVLIGGLEWNDNTVRVSKNLQRFAWRSSNENGGPPLTPKQKRKMARTARSKV

SEQ ID NO: 41 - DNA, 890 bp, Influenza A virus; identical to SEQ ID NO: 27

SEQ ID NO: 42 - protein, 230 aa, Influenza A virus
MDSNTVSSFQVDCFLWHVRKRFADQELGDAPFLDRLRRDQKSLRGRGSTLGLDIRTATREGKHIVERILEEESDEALKMTIASVPASRYLTEMTLEEMSRDWLMLIPKQKVTGPLCIRMDQAVMGKTIILKANFSVIFNRLEALILLRAFTDEGAIVGEISPLPSLPGHTDEDVKNAIGVLIGGLEWNDNTVRVSETLQRFTWRSSDENGRSPLPPKQKRKVERTIEPEV

SEQ ID NO: 43 - DNA, 2329 bp, Influenza A virus
aaaagcaggtcaattatattcaatatggaaagaataaaagagctaaggaatttgatgtcacaatctcgcactcgcgagatacttaccaaaactactgtagaccacatggccataatcaagaaatacacatcaggaagacaggagaaaaacccatcacttaggatgaaatggatgatggcaatgaaatacccaattacagctgataaaaggataacggaaatgattcctgaaagaaatgagcatggacagacattatggagtaaggtgaatgatgccggatcagaccgagtgatggtatcacccctggctgtgacatggtggaacagaaatggaccagtggcaagtactattcactatccaaaaatctacaaaacttactttgaaaaggttgaaaggttaaaacaaggaacctttggccctgtacactttagaaaccaagtcaaaatacgccgaagagtcgacataaatcctggtcatgcagacctcagcgccaaggaggcacaggatgtaattatggaagttgttttccctaatgaagtaggagccagaatactaacatcagaatcgcaattaacgataaccaaggagaaaaaagaagaactccagaattgcaaaatttcccctttgatggttgcatacatgttagagagggaacttgtccgcaaaacaagatttctcccggttgcaggtggaacaagcagtgtgtacattgaagttttgcatttaacacaggggacatgctgggagcagatgtacactccaggtggggaggtgaggaatgatgatgttgatcaaagcctaattattgctgctaggaacatagtgagaagagctgcagtatcagcagatccactagcatccttattagaaatgtgccatagcacacagattggtggaacaaggatggtggatattctcaggcaaaatccaacagaagaacaagctgtggacatatgcaaagcagcaatggggctgagaatcagctcatccttcagttttggcggattcacatttaagagaacaagtggatcatcagtcaaaagggaggaagaagttctcacgggcaatctgcaaacattgaaactaaccgtgcatgagggatatgaagagttcacaatggttgggaaaagggcaacagctatactcagaaaagcaaccaggagattgattcaactaatagtgagtggaagagacgaacaatcaatagtcgaagcaatagttgtagcaatggtattctcacaagaagattgcatggtaaaagcagttagaggtgatctgaatttcgttaatagagcgaatcagcggttgaatcccatgcatcaactattgagacattttcagaaggatgctaaagtacttttcttaaattggggagttgaacctattgacaatgtgatgggaatgattgggatattacctgatatgactccaagtaccgagatgtcaatgagaggagtgagagtcagcaaaatgggtgtagatgaatactccaatgctgaaagggtagtggtaagcattgaccgttttttgagagtccgggaccaaagaggaaatgtactactgtctccagaggaagtaagtgaaacacaagggacagagaaactgacaataacttactcttcatcaatgatgtgggagattaatggccctgagtcagtcttgatcaatacctatcagtggatcatcagaaactgggagactgttaaaattcagtggtctcagaatcctacgatgctgtacaataaaatggaatttgaaccatttcagtctctagtccccaaggccattagaggccaatacagtgggtttgttagaactctattccaacaaatgagggatgtgcttgggacttttgacacaactcagataataaaacttcttccctttgcagccgctcctccaaagcaaagcagaatgcaattctcgtcattaactgtgaatgtgaggggatcaggaatgagaatacttgtgaggggtaattctccagtattcaactacaacaagactaccaagagactcacagtcctcggaaaggatgctggcactttaactgaagacccagatgaaggcacagctggagtggaatctgcggttctaaggggattcctcattttaggcaaagaagatagaagatatgggccagcattaagcatcaatgaattgagcaatcttgcgaaaggagaaaaagctaatgtgctaattgggcaaggggatgtagtgttggtaatgaaacgaaaacgggactctagcatacttactgacagccagacagcgaccaaaagaattcggatggccatcaattaatttcgaataatttaaaaacgacctt

SEQ ID NO: 44 - DNA, 2337 bp, Influenza A virus
aaagcaggcaaaccatttgaatggatgtcaatccgacattacttttcttaaaagtgccagcacaaaatgctataagcacaacttttccttatactggtgaccctccttacagccatgggacaggaacagggtacaccatggatacagtcaacaggacacaccagtactcagaaagaggaagatggacaaaaaataccgaaacgggagcaccgcaacttaacccaattgatggtcccttaccggaagacaatgaaccaagtggctatgcccaaacagattgtgtattagaagcaatggctttccttgaagaatcccatcccggtatctttgaaaactcttgtattgaaacaatggaggttgttcaacaaacaagggtggacaaactgacacaaggcagacagacctatgactggactctaaataggaaccagcctgctgccacagcattggcaaacactatagaagtattcagatcaaacggcctcatagcaaatgaatctggaaggctaatagacttccttaaagatgtaatggagtcgatggacagaggcgaagtagaggtcacaactcattttcaaagaaagaggagagtgagagacaatgtaactaaaaaaatggtgacccaaagaacaataggcaaaaagaaacataaattagacaaaagaagttacctaattagggcattaaccctgaacacaatgaccaaagatgctgagagggggaaactaaaacgcagagcaattgcaaccccaggaatgcaaataagggggtttgtatactttgttgagacactggcaagaagcatatgtgaaaagcttgaacaatcaggattgccagttggaggaaatgagaagaaagcaaagttagcaaatgttgtaaggaagatgatgaccaactcccaggacactgaaatttctttcaccataaccggagacaacacaaaatggaacgaaaatcaaaaccctagaatgttcttggccatgatcacatatataaccaaaaatcagcctgaatggttcagaaatattctaagtattgctccaataatgttttcaaacaagatggcgagactaggtaaggggtacatgtttgaaagcaagagtatgaaactgagaactcaaatacctgcagagatgctagccaacatagacttgaaatatttcaatgattcaactaaaaagaaaattgaaaaaatccgaccattattaatagatggaactgcatcattgagtcctggaatgatgatgggcatgttcaatatgttgagcaccgtattgggcgtctccattctgaatcttgggcaaaagagatacaccaagactacttactggtgggatggtcttcaatcgtctgatgattttgctttaattgtgaacgcacccaactatgcaggaattcaagctggagttgacaggttttatcgaacctgtaagctgctcggaattaatatgagcaaaaagaagtcttacataaacagaacaggtacctttgaattcacgagctttttctatcgttatgggtttgttgccaatttcagcatggagcttcctagttttggggtgtctggggtcaatgaatctgcagacatgagtattggagtcactgtcatcaaaaacaatatgataaacaatgaccttggcccagcaactgctcaaatggcccttcagttatttataaaagattacaggtacacttatcgatgccaccgaggtgacacacaaatacaaacccgaagatcatttgagataaagaaactatgggaccaaacccgctcaaaagctggactgttggtctctgatggaggccccaatttgtataacattagaaatctccatattcctgaagtttgcttgaaatgggagttgatggatgaggattaccaggggcgtttatgcaacccattaaacccgtttgtcagccataaagagattgaatcagtgaacagtgcagtgataatgccggcacatggtccagccaaaaatatggagtatgacgctgttgcaacaacacactcctgggtccccaaaagaaatcgatccattttgaacacgagccaaagggggatacttgaagatgagcaaatgtatcagaggtgctgcaatttatttgaaaaattcttcccaagtagctcatacagaagaccagttggaatatccagtatggtagaggctatggtctcaagagcccgaattgatgcacggattgatttcgaatctggaaggataaagaaagaggaatttgctgagatcatgaagatctgttccaccattgaagacctcagacggcaaaaatgaggaatttggcttgtccttcatgaaaaaatgccttgtttctact

SEQ ID NO: 45 - DNA, 2217 bp, Influenza A virus
tactgattcaaaatggaagattttgtacgacaatgcttcaatccgatgattgtcgagcttgcagaaaaggcaatgaaagagtatggagaggacctgaaaatcgaaacaaacaaatttgcagcaatatgcacccacttggaagtgtgcttcatgtattcagattttcatttcataaatgagcaaggcgaatcaataatagtagagcctgaggacccaaatgcacttttaaagcacagatttgagataatagagggacgagatcgtacaatggcatggacagttgtaaacagtatttgcaacaccacaggagctgagaaaccaaagtttctgccagatctgtatgattacaaagagaatagattcatcgagattggagtgacaaggagggaagttcacatatactatctggaaaaggccaacaaaattaaatctgagaagacacacattcacattttctcattcaccggcgaagaaatggccacaaaggctgattacactctcgatgaagaaagcagagctaggattaaaaccagattgttcaccataagacaagaaatggcaagcagaggtctttgggactcctttcgtcagtccgaaagaggcgaagaaacaattgaagaaagatttgaaatcacagggacaatgcgcaggctcgctgaccaaagccttccgccgaacttctcctgcattgagaattttagagcctatgtggatggatttgaaccgaacggctacattgagggcaagctttctcaaatgtccaaagaagtaaatgctagaattgagccttttttgaaaacaacacctcgaccaattagacttccgaatgggcctccttgttttcagcggtcaaaattcctgctgatggattctttaaaattaagcattgaggatccaaatcatgaaggtgaggggataccactatatgatgcaatcaagtgtatgaggacattctttggatggaaagaacccactgttgtcaagccacacgagaagggaataaatccaaattatctgttgtcgtggaagcaagtattggaagagctgcaggacattgagagtgaggagaagattccaagaacaaaaaacatgaaaaaaactagtcagctaaagtgggcacttggtgagaacatggcaccagagaaggtggattttgatgactgtaaagatataagcgatttgaagcaatatgacagtgacgaacctgaattaagatcattttcaagttggatccagaatgagttcaacaaggcatgcgagctgaccgattcaatctggatagagcttgatgagattggagaagatgtggctccgattgaacacattgcaagcatgagaaggaattacttcacagctgaggtgtcccactgcagagccacagaatatataatgaagggggtatacattaatactgctttgctcaatgcatcctgtgcagcaatggatgatttccaactaattcccatgataagcaaatgtagaactaaagagggaaggagaaagaccaatttgtacggcttcatcgtaaaaggaagatctcacttaaggaatgacaccgatgtggtaaactttgtgagcatggagttttccctcactgacccaagacttgagccacacaaatgggagaagtactgcgttcttgagataggagatatgcttctaaggagtgcaataggccaagtgtcaaggcccatgttcttgtatgtaaggacaaatggaacctcaaaaattaaaatgaaatggggaatggagatgaggcgttgcctcctccaatccctccaacaaatagagagcatgattgaagctgagtcctctgtcaaagagaaagacatgacaaaagagttttttgagaataaatcagaaacatggcccattggagagtcaccaaaaggagtggaagaaggttccattgggaaagtatgcaggacactgttggctaagtcagtattcaatagcctgtatgcatctccacaattagaaggattttcagctgagtcaagaaagttgctcctcattgttcaggctcttagggacaatctggaacctgggacctttgatcttggggggctatatgaagcaattgaggagtgcctgattaatgatccctgggttttgcttaatgcttcttggttcaactccttcctaacacatgcattgagatagctggggcaatgctactatttgctatccatactgtccaaaaaagtaccttgtttc

SEQ ID NO: 46 - DNA, 1771 bp, Influenza A virus
gcaaaagcaggggaaaataaaacaaccagaatgaaagtaaaactactggtcctgttatgcacatttacagctacatatgcagacacaatatgtataggctaccatgctaacaactcgaccgacactgttgacacagtacttgaaaagaatgtgacagtgacacactctgtcaacctgcttgagaacagtcacaatgggaaactatgttcattaaaaggaatagccccactacaattgggtaactgcagcgttgccgggtggatcttaggaaacccagaatgcgaattactgatttccaaggagtcatggtcctacattgtagaaaaaccaaatcctgagaatggaacatgttacccagggcatttcgctgactatgaggaactgagggagcaattgagttcagtatcttcatttgagaggttcgaaatattccccaaagaaagctcatggcccaaccacaccgtaaccggagtgtcagcatcatgctcccataatggggaaagcagtttttacagaaatttgctatggctgacggtgaagaatggtttgtacccaaacttgagcaagtcctatgcaaacaacaaagaaaaagaagttcttgtactatggggtgttcatcacccgccaaacatagttgaccaaaagaccctctatcgtacagaaaatgcttatgtttctgtagtgtcttcacattatagcagaaaattcaccccagaaatagccaaaagacccaaagtaagagatcaagaaggaagaatcaactactactggactctgcttgaacccggggatacaataatatttgaggcaaatggaaatctaatagcgccaagatatgctttcgcactgagtagaggctttggatcaggaatcatcaactcaaatgcaccaatggataaatgtgatgcgaagtgccaaacacctcggggagctataaacagcagtcttcctttccagaacgtacacccagtcacaataggagagtgtccaaagtatgtcaggagtgcaaaattaaggatggttacaggactaaggaacatcccatccattcaatccagaggtttgtttggagccattgccggtttcattgaaggggggtggactggaatggtagatggttggtatggttatcatcatcagaatgagcaaggatctggctatgctgcagatcaaaaaagcacacaaaatgccattaatgggattacaaacaaggtgaattctgtaattgagaaaatgaacactcaattcacagcagtgggcaaagaattcaacaaattggaaagaaggatggaaaacttgaataaaaaagttgatgatgggtttatagacatttggacatataatgcagaactgttggttctactggaaaatgaaaggactttggatttccatgactccaatgtgaagaatctgtatgagaaagtaaaaagccagttaaagaataatgctaaagaaataggaaatgggtgttttgaattctatcacaagtgtaacgatgaatgcatggagagtgtaaagaatggaacctatgactatccaaaatattccgaagaatcaaagttaaacagggagaaaattgatggagtgaaattggaatcaatgggagtctatcagattctggcgatctactcaacagtcgccagttctctggttcttttggtctccctgggggcaatcagcttctggatgtgttccaatgggtctttgcagtgtagaatatgcatctaagaccagaatttcagaaatataaggaaaaacacccttgtttcta

SEQ ID NO: 47 - DNA, 1554 bp, Influenza A virus
agcaaaagcagggtagataatcactcactgagtgacattaaagtcatggcgtcccaaggcaccaaacggtcttacgaacagatggagactgatggggaacgccagaatgcaactgaaatcagagcatccgtcggaagaatgattggtggaattgggcgattctacatccaaatgtgcaccgagcttaagctcaatgattatgagggacggctgatccagaacagcttaacaatagagagaatggtgctctctgcttttgatgagaggagaaataaatatctagaggaacatcccagcgcggggaaagatcctaagaaaactggaggacccatatacaaaagagtagatggaaagtgggtgagggaactcgtcctttatgacaaagaagaaataaggcggatttggcgccaagccaacaatggtgatgatgcaacggctggtttgactcacattatgatctggcattctaatttgaatgatacaacttatcagaggacaagagctcttgtccgcaccggaatggatcccaggatgtgctctttgatgcaaggttcgactctccctagaagatctggagcagcaggcgccgcagtcaaaggagttgggacaatggtattggaattaatcaggatgatcaaacgtgggatcaatgaccgaaacttctggaggggtgagaatggaagaaaaacaaggattgcttatgagagaatgtgcaacattctaaaaggaaaatttcaaacagctgcacaaaaagcaatgatggaccaagtgagagaaagccggaacccaggaaatgctgagatcgaagatctcacttttctggcacggtctgcactcatattgagaggatcagttgctcacaagtcttgcctgcctgcctgtgtgtatggaccagccgtagccagtgggtatgacttcgaaaaagagggttactctttggtaggagtagaccctttcaaactgcttcaaaccagtcaggtatacagtctaattagaccaaacgagaatcccgcacacaagagccagttggtgtggatggcatgcaattctgctgcatttgaagatctaagagtgtctagcttcatcagaggaacaagagtacttccaagggggaagctctccactagaggagtacaaattgcttcaaatgaaaacatggatgctattgtgtcaagtactcttgaactgagaagcagatactgggccataagaaccagaagtggagggaacactaatcaacaaagggcctctgcgggccaaatcagcacacaacctacgttttctgtgcagagaaacctcccatttgacaaagcaaccatcatggcagcattctctgggaatacagagggaagaacatcagacatgagggcagaaatcataaagatgatggaaagtgcaagaccagaagaagtgtccttccaggggcggggagtctttgagctctcggacgaaagggcaacgaacccaatcgtgccctcctttgacatgagtaatgaaggatcttatttcttcggagacaatgcagaggagtacgacaattaatgaaaaataccc

SEQ ID NO: 48 - DNA, 1438 bp, Influenza A virus
aaagcaggagtttaaaatgaacccaaatcaaaagataataaccattggatcaatcagtatagcaatcggaataattagtctaatgttgcaaataggaaatattatttcaatatgggctagtcactcaatccaaactggaagtcaaaacaacactggaatatgcaaccaaagaatcatcacatatgaaaacagcacctgggtgaatcacacatatgttaatattaacaacactaatgttgttgctggagaggacaaaacgtcagtgacattggccggcaattcatctctttgttctatcagtggatgggctatatacacaaaagacaacagcataagaattggctccaaaggagatgtttttgtcataagagaacctttcatatcatgttctcacttggaatgcagaaccttttttctgacccaaggcgctctattaaatgacaaacattcaaatgggaccgtaaaggacagaagtccttatagggccttaatgagctgtcctctaggtgaagctccgtccccatacaattcaaagttcgaatcagttgcatggtcagcaagcgcatgccatgatggcatgggctggttgacaatcggaatttctggtccagacaatggagctgtggctgtactaaaatacaacggaataataactggaaccataaaaagttggaaaaagcaaatattaagaacacaagagtctgaatgtgtctgtatgaacgggtcatgtttcaccataatgaccgatggcccgagtaataaagccgcctcgtacaaaattttcaagatcgaaaaggggaaggttactaaatcaatagagttgaatgcacccaatttttattacgaggaatgctcctgttacccagacactggcatagtgatgtgtgtatgcagggacaactggcatggttcaaatcgaccttgggtgtcttttaatcaaaatttggattatcaaataggatacatctgcagtggagtgtttggtgacaatccgcgtcccgaagatggagagggcagctgcaatccagtgactgttgatggagcagacggagtaaaagggttttcatacaaatatggtaatggtgtttggataggaaggaccaaaagtaacagacttagaaaggggtttgagatgatttgggatcctaatggatggacaaataccgacagtgatttctcagtgaaacaggatgttgtagcaataactgattggtcagggtacagcggaagtttcgtccaacatcctgagttgacaggattggactgtataagaccttgcttctgggttgagttagtcagagggctgcctagagaaaatacaacaatctggactagtgggagcagcatttctttttgtggcgttaatagtgatactgcaaactggtcttggccagacggtgctgagttgccgttcaccattgacaagtagttcgttgaa

SEQ ID NO: 49 - DNA, 1016 bp, Influenza A virus
gcaaaagcaggtagatattgaaagatgagtcttctaaccgaggtcgaaacgtacgttctctctatcatcccgtcaggccccctcaaagccgagatcgcacagagacttgaagatgtatttgctggaaagaataccgatcttgaggctctcatggagtggctaaagacaagaccaatcctgtcacctctgactaaggggattttaggatttgtgttcacgctcaccgtgcccagtgagcgaggactgcagcgtagacgctttgtccaaaatgcccttaatgggaatggggatccaaataatatggacagagcagtcaaactttatcgaaagcttaagagggagataacattccatggagccaaagaaatagcactcagttattctgctggtgcacttgccagttgtatgggactcatatacaacaggatgggggctgtgaccaccgaatcagcatttggccttatatgtgcaacctgtgaacagattgccgactcccagcataagtctcacaggcaaatggtaacaacaaccaatccattaataagacatgagaacagaatggttctggccagcactacagctaaggctatggagcaaatggctggatcgagcgaacaagcagctgaggccatggaggttgctagtcaggccaggcagatggtgcaggcaatgagagccattgggactcatcctagctctagcactggtctgaaaaatgatctccttgaaaatttacaggcctatcagaaacgaatgggggtgcagatgcaacgattcaagtgatcctcttgttgttgccgcaagtataattgggattgtgcacttgatattgtggattattgatcgccttttttccaaaagcatttatcgtatctttaaacacggtttaaaaagagggccttccacggaaggagtaccagagtctatgagggaagaatatcgagaggaacagcagaatgctgtggatgctgacgatgatcattttgtcagcatagagctggagtaaaaaactacct

SEQ ID NO: 50 - DNA, 880 bp, Influenza A virus
agcgaaagcagggtggcaaagacataatggattcccacactgtgtcaagctttcaggtagattgcttcctttggcatgtccgcaaacaagttgcagaccaagatctaggcgatgcccccttccttgatcggcttcgccgagatcagaagtctctaaagggaagaggcagcactctcggtctgaacatcgaaacagctacttgtgttggaaagcaaatagtagagaggattctgaaagaagaatccgatgaggcacttaaaatgaccatggcctccgcacttgcttcgcggtacctaactgacatgactgttgaagaaatgtcaagggactggttcatgctcatgcccaagcaaaaaatggctggccctctttgtgtcagaatggaccaggcaataatggataagaacatcatactgaaagcgaatttcagtgtgatttttgaccggttggagaatctggcattattaagggcttttaccgaagagggagcaattgttggcgaaatttcaccattgccttcttttccaggacatactaatgaggatgtcaaaaatgcaattggggtcctcatcgggggacttgaatggaatgataacacagttcgagtctctgaaactctacagagattcgcttggagaagcagtaatgagactgggggacctccattcactacaacacagaaacggaaaatggcgggaacaactaggtcagaagtttgaagaagtaagatggctgattgaagaagtgaggcataaattgaagacgacagagaatagttttgagcaaataacatttatgcaagcattacagctattatttgaagtggaacaagagattagaacgttttcgtttcagcttatttagtgataaaaaacaccct